(19) United States
(12) Patent Application Publication
(10) Pub. No.: US 2026/0207768 A1
(43) Pub. Date: Jul. 23, 2026

(54) NOVEL LINKER DRUGS COMPRISING PHOSPHOANTIGENS, NOVEL CONJUGATES AND THEIR USE IN THERAPY

(71) Applicant: Byondis B.V., Nijmegen (NL)

(72) Inventors: Dennis WAALBOER, Nijmegen (NL); Ronald ELGERSMA, Nijmegen (NL)

(21) Appl. No.: 19/141,740

(22) PCT Filed: Dec. 20, 2023
(86) PCT No.: PCT/EP2023/086808

§ 371 Date: Jun. 20, 2025

(30) Foreign Application Priority Data
Dec. 22, 2022 (EP) 22215957.6

(51) Int. Cl. A61K 47/68; A61K 47/54; A61P 35/00

(52) U.S. Cl. CPC A61K 47/6889; A61K 47/545; A61K 47/6803; A61K 47/6851; A61P 35/00

(57) ABSTRACT

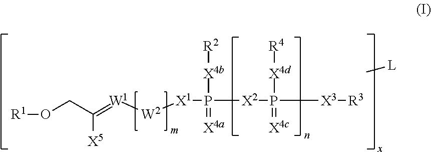

The present invention relates to novel linker drug compounds based on specific phosphoantigens (pAg) with the general structure reflected in formula (I), wherein L represents a linking moiety (linker) and, W¹, W², X¹⁻⁵, x, m, n and R¹⁻⁴ are as defined in the specification. Further provided are conjugates comprising a targeting moiety, preferably a tumor-targeting antibody or antigen binding fragment thereof, covalently linked to a linker drug compound according to the invention. Such conjugates can be used, for example, in the treatment of diseases such as cancer, infection, or autoimmune disease.

### FIELD OF THE INVENTION

The present invention relates to novel linker drug compounds comprising one or more phosphoantigen moieties covalently bound to a linking moiety, for use in the manufacture of conjugates.

The invention further relates to conjugates comprising a targeting moiety, for example an antibody or a binding fragment thereof, linked to said linker drug compound, pharmaceutical compositions comprising said conjugates, and the use thereof in the treatment of diseases, such as cancer, infectious diseases and autoimmune diseases, optionally in combination with other therapeutic agents.

### BACKGROUND OF THE PRESENT INVENTION

Conventional methods to treat cancer involve surgery, chemotherapy with cytotoxic agents and radiation therapy, or a combination of these treatments. Due to their toxic and non-specific nature, treatment with cytotoxic agents or radiation often lead to severe side effects. Since it was discovered that the immune system plays an important role in eradicating neoplastic cells, more recent cancer therapies aim to use components of the immune system as a tool to treat cancer.

One approach used in cancer immunotherapy is to target “immune checkpoints”, such as T-lymphocyte associated protein 4 (CTLA-4) or programmed cell death protein 1 (PD-1), aiming to activate anti-tumor immune responses in patients with cancer. Both CTLA-4 and PD-1 are proteins involved in negative feedback systems, which function to restrain immune cell activation. Tumor cells can escape from the immune system by “abusing” this suppression mechanism by overexpressing immune-checkpoint ligands on their surface, to protect themselves from an attack by cells of the immune system. Activation of immune checkpoints, by interaction with their ligands, leads to T-cell inactivation and exhaustion. Immune checkpoint inhibitors, such as antibodies directed against immune checkpoints or their ligands, are a new class of anti-cancer drugs that block the immune checkpoints overexpressed on cancer cells. Examples of approved immune checkpoint inhibitors are ipilimumab (blocking CTLA-4; brand name Yervoy®, produced by BMS), approved in 2011 for treatment of melanoma, PD-1 antibody nivolumab (sold under the brand name Opdivo® and developed by BMS) and pembrolizumab (brand name Keytruda®, another PD-1 inhibitor, produced by Merck). While checkpoint inhibitors can reinvigorate an anti-tumor response, activated immune cells can also attack normal tissue, leading to immunological adverse side-effects.

Another approach to cancer therapy involves the use of Antibody-Drug Conjugates (ADCs). ADCs combine the specificity of a monoclonal antibody for a tumor specific antigen with the cell killing activity of a chemical cytotoxic agent. The antibody of an ADC acts as a targeting agent and carrier for the cytotoxic payload. The binding of the antibody to its target effectuates efficient uptake of the ADC, with its cytotoxic payload, into the target tumor cells. The cytotoxic payload may be an inactive precursor (prodrug) of a cytotoxic agent, grafted onto the antibody via a linker which is stable in circulation, and is cleaved after being internalized into the tumor cell, for example by intracellular proteases. The cleavage of the linker may trigger the release of the active, cytotoxic form of the payload in the tumor cell. ADCs have the advantage that toxic, and non-specific side-effects on healthy tissue, can be greatly reduced. ADCs that have been clinically approved include, gemtuzumab (anti-CD33) ozogamicin (Mylotarg®; Wyeth Pharmaceuticals, a subsidiary of Pfizer), brentuximab (anti-CD30) vedotin (Adcetris®; Seattle Genetics/Millennium Pharmaceuticals), (ado-) trastuzumab (anti-HER2) emtansine (Kadcyla®; Genentech/Roche), inotuzumab (anti-CD22) ozogamicin (Besponsa®; Wyeth Pharmaceuticals, a subsidiary of Pfizer), enfortumab (anti-nectin-4) vedotin (Padcev™; Astellas Pharma/Seattle Genetics), fam-trastuzumab deruxtecan (Enhertu®; Daiichi Sankyo/AstraZeneca), polatuzumab (anti-CD79b) vedotin (Polivy™; Genentech/Roche) and sacituzumab (anti-TROP-2) govitecan (Trodelvy™; Immunomedics). Many more are in clinical development.

Yet another approach to cancer therapy is immunotherapy using therapeutic compounds that activate the immune system, in particular T-cells, to attack and destroy tumor cells. Such therapeutic compounds may be agonists of immune cell receptors and can be large molecules or relatively small chemical structures. An example of such compounds are ligands activating Toll Like Receptors (TLRs). Several TLR ligands have been approved for cancer therapy. The first approved TLR ligand (TLR agonist) form part of an attenuated strain of Mycobacterium bovis called Bacillus Calmette-Guérin (BCG). First developed as a tuberculosis vaccine, BCG contains active TLR2/4 ligand and has been used as a treatment for bladder cancer. Other approved TLR ligands are the TLR4 ligand monophosphoryl lipid A (MPLA) and the small molecule TLR7 agonist imiquimod, an imidazoquinoline.

TLR ligands have also been used in immunoconjugates. Such immunoconjugates comprise an antibody specific for a tumor antigen as targeting vehicle for a TLR ligand, with the aim to induce localized activation of cells of the immune system in the tumor microenvironment. Immunoconjugates, for the treatment of breast cancer, wherein TLR agonist were coupled to anti-HER antibodies are described in WO2017/072662 (Novartis A. G.). A further anti-HER conjugates with a TLR8 agonist payload were developed by Silverback Therapeutics (ImmunoTAC™ SBT6050). Bolt Therapeutics (WO2020/047187) and Ackerman et al., 2021, Nature Cancer, Vol. 2 (8), 18-33, also describe TLR immunoconjugates, comprising a tumor-targeting monoclonal antibody, conjugated to a TLR 7/8 agonist (T785) via a non-cleavable linker; The tumor targeting antibody bound to a tumor antigen activates antigen presenting cells present in the tumor microenvironment (TME) via Fc effector functions, while the TLR agonist bound thereto directly stimulates APCs through their TLR receptors, which in turn promotes anti-tumor immunity.

A specific subset of T-cells known to display cytotoxicity against cancer cells are gammadelta T-cells. (T-cells with T-cells receptors (TCRs) composed of gamma and delta chains). Gamma delta T-cells are considered a unique subset of T-lymphocytes due to their ability to effectuate a rapid, innate-like immune response to infection and to tumor cells. Tumor-infiltrating gammadelta T-cells (γδ T-cells) were found in many different malignancies (Gentles et al., Nature Medicine, 2015, 21 (8), 938-945). Gammadelta T-cells, or more specifically; Vgamma9Vdelta2 T-cells (Vy9V82 T-cells), which form a major subset of gammadelta T cells, can be activated by a specific set of antigens known as “phosphoantigens”. Naturally occurring phosphoantigens are low molecular alkyl pyrophosphates, such as 4-hydroxy-3-methyl-but-2-enyl-pyrophosphate (HMBPP) and isopentenyl pyrophosphate (IPP). These natural phosphoantigens are produced by pathogenic cells where HMBPP is the immediate precursor of IPP (HMBPP is a pathogenic phosphate antigen that does not occur in humans). Bacteria and parasites can produce isoprenoid precursors using a mevalonate-independent pathway (MEP) pathway or 2-C-methyl-D-erythritol 4-phosphate/1-deoxy-D-xylulose 5-phosphate (MEP/DOXP) pathway), resulting in the biosynthesis of the isoprenoid precursor IPP. In humans pAg production is driven by the mevalonate pathway.

In contrast to TLR agonists, phosphoantigens do not work directly on receptors displayed on myeloid cells or T-cells. It is believed that intracellular (e.g. within a cancer cell) binding of a phosphoantigen to an intracellular domain of a cell surface molecule, butyrophilin 3A1 (BTN3A1) causes conformational changes in relation to the extracellular portion of the BTN3A1 complex, which also includes a role for BTN2A1 (Sandstrom A, et al., 2014, Immunity, 40 (4), 490-500, doi: 10.1016/j.immuni.2014.03.003).

The conformational changes in the extracellular BTN3A1/BTN2A1 complex result in binding to the gammadelta TCR, which results in cytokine production and killing of the tumor/pathogenic cell by the activated gammadelta T-cell (Rigau et al., Science, 2020, 367, 642). The activation of gammadelta T-cells using phosphoantigens as therapeutic agents is thus indirect; The phosphoantigen acts from within a cell (e.g. a tumor cell or infected cell), to effectuate a conformational change in the extracellular BTN3A1/BTN2A1 complex on the surface of said cell, which in turn provides an activating signal to gammadelta TCRs on gammadelta T-cells. The gammadelta T-cells will in turn exert their cell killing effect on the tumor cells or infected cells.

Because pyrophosphate HMBPP has poor pharmacokinetic properties (it is rapidly hydrolyzed in plasma), (nitrogenous) bisphosphonate analogs have been developed, as well as (monophosphate) prodrug forms that are converted to active phosphoantigens after they are administered to a subject. In phosphoantigen-prodrugs, the negatively charged non-binding oxygen atoms of the phosphonate group(s) are protected with neutral groups to increase, for example, diffusion over the cell membrane. The protecting groups are removed once inside the cell to release the active phosphoantigen. Another approach to improve the half-life in circulation of phosphoantigens (in particular of bisphosphonate phosphoantigens) is described in WO2012/042024. Phosphoantigens were complexed to nanoparticles with inorganic and lipid nano vectors, serving as delivery vehicles for the phosphoantigens. It was mentioned that the resulting nanoparticles can be coated with targeting ligands on their surface, to target specific cells. Examples mentioned include molecules that induce targeting to cancer cells, such as antibodies. The use of human transferrin was exemplified.

Compounds with cellular pAg activity are believed to be able to display their activity directly, through binding to a pAg receptor in a target cell (“direct pAgs”). This receptor is believed to be the intracellular domain of a cell surface molecule, butyrophilin 3A1 (BTN3A1). Natural phosphoantigens include pyrophosphates (diphosphates) such as HMBPP and IPP. Known analogs of natural phosphoantigens include bromohydrin pyrophosphate (BrHPP) and pyrophosphonates such as C-HMBPP, which is the pyrophosphonate equivalent of the naturally occurring HMBPP. The natural pAg HMBPP is produced by pathogenic bacteria. It was found that the allylic alcohol in natural pAgs such as HMBPP, is important for BTN3A1 binding and maximal pAg activity. Direct pAgs, such as HMBPP bind directly to BTN3A1 in its intracellular B30.2 domain. Further analogs of HMBPP, for example halohydrins such as BrHPP, IHPP and CIHPP are also known in the art (Wiemer et al., 2020, Chem. Med. Chem., 15, 1030-1039).

Phosphoantigen analogs with alleged increased potency, phosphoramidite esters, are described in WO2005/05258 (Innate Pharma), for example N-HDMAPP, wherein the isoprene unit present in natural HMBPP is linked to the pyrophosphate through an NH group.

Other compounds show indirect pAg activity, through accumulation of IPP. Such compounds can be referred to as “indirect pAgs”. Indirect pAgs act on pathways that increase cellular levels of (endogenous) direct pAgs, such as IPP and concomitant activation of Vγ9Vδ2 T cells. In contrast to direct pAgs, indirect pAgs do not interact directly with the butyrophilin receptors in target cells, nor are they pAg precursors (compounds that are converted, enzymatically or chemically, to direct pAgs). Indirect pAgs can be compounds that, for example, inhibit downstream enzymes, such as farnesyl pyrophosphate synthase (FPPS). Inhibition of FPPS blocks use of IPP, and leads to accumulation of IPP in a cell. Known FPPS inhibitors are aminobisphosphonates (N-BPs), such as zoledronate. (Wiemer et al., 2020, Chem. Med. Chem., 15, 1030-1039; Park et al., 2021, Frontiers in Chemistry, Vol. 8, Article 612728).

Further examples of bisphosphonates with a nitrogen or amino-group in one of the substituents on the central carbon atom, believed to increase the potency of the bisphosphonate (Drake et al., Mayo Clin. Proc., 2008, 83 (9), 1032-1045), include, alendronate, risedronate, ibandronate, pamidronate, neridronate and olpadronate.

Aminobisphosphonates (N-BPs), such as zoledronate, pamidronate and alendronate, are also known as “bone targeting agents”, because of their ability to specifically bind to hydroxyapatite (HA) (Farrell et al., 2018, Bone Reports, 9, 47-60). Alendronate was also conjugated to trastuzumab, with the aim to target trastuzumab to bone metastasis, using alendronate as the bone targeting agent (Tian et al., 2021, Sci.Adv., 7, 2-11). Due to its negative charge, alendronate has a high affinity for HA, resulting in preferential binding to the bone. Tian et al, thus proposed the use of negatively charged aminobisphosphonates like alendronate as targeting agent for an antibody for treatment of bone-related diseases.

Phosphoantigens have been tested for use in cancer therapy, with the aim to promote the cytotoxic effect of gammadelta T-cells on tumor cells, either in vivo or by expanding gammadelta T-cells in vitro together with antigen presenting cells, for administration to a subject. Synthetic phosphoantigens, such as BrHPP (Phosphostim, manufactured by Innate Pharma) and Zoledronate (Novartis) have been the subject of clinical testing in patients with cancer. Phosphoantigens that were the subject of clinical testing showed an acceptable safety profile. However, their efficacy was general not sufficient. (Sebestyen et al., Nature Reviews Drug Discovery, 2020, 19 (3), 169-184).

Finding an acceptable therapeutic window for such treatment may be greatly improved by more robust, selective, as well as effective, ways to deliver phosphoantigens to cells (over) expressing butyrophilin (BTN3A1/BTN2A1) complexes, such as tumor- or pathogenic cells.

### BRIEF DESCRIPTION OF THE PRESENT INVENTION

The present invention provides more effective and selective ways of using phosphoantigens in treatment of, for example, cancer. The present invention relates to linker-drug compound with the general structure reflected in formula (I)

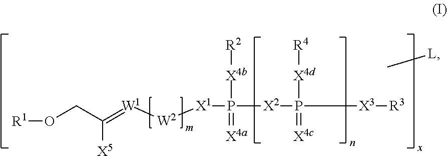

wherein L represents a linking moiety,
W¹ is N, CH or CF, preferably CH;
W² is CH₂, CHF, CF₂ or O;
X¹ is O, S, NH, CH₂, CHF or CF₂;
X² is O, CH₂, CHF or CF₂;
X³ is absent or O or NH;
each of X^{4a-d} is independently selected from O and S;
X⁵ is
   H, halogen (F, Cl, Br, I) or nitril (CN) or
   ethenyl, ethynyl, ethyl, optionally substituted with one or more of the following groups: F, CH₃, CH₂F, CHF₂, CF₃, or
   C₃-C₄ cycloalkyl, C₃-C₄ cycloalkenyl, allyl, propynyl, O—CH₃, S—CH₃, optionally substituted with one or more fluorine substituents, or
   CHR¹OR², CHR¹SR², CHO, CO₂R¹, CONR¹R², where R¹ and R² are independently selected from H, CH₃, CH₂F, CHF₂, CF₃, CH₂CH₃;
x is an integer ranging from 1-5;
m is 1, 2 or 3;
n is 0, 1 or 2;
R¹ is H or a connection to the linking moiety (L) or a prodrug moiety;
R² is H or a connection to the linking moiety (L) or Cat+ or a prodrug moiety;
R³ is H or a connection to the linking moiety (L) or Cat+ or a prodrug moiety;
R⁴ is H or a connection to the linking moiety (L) or Cat+ or a prodrug moiety; or, when n is 0, R³ and R² are connected by a C₁₋₆ (hetero)alkyl group or;
when n is 1 or 2, R³ and R⁴ are connected by a C₁₋₆ (hetero)alkyl group.

Preferably X⁵ is H or halogen (C₁, F, I or Br), most preferably Br or Cl.

When W¹ is CH, R¹ preferably is H or a connection to the linking moiety (L). When X³ is O, R³ preferably is a connection to a linking moiety and R¹ is H. In linker drug compounds according to the invention, when W² is CH₂, preferably m is 1. X¹ preferably is CH₂.

In a preferred embodiment, X³ is O, R³ is a connection to a linking moiety, W¹ is CH, R¹ is H, W² is CH₂ and m is 1, and X¹ is CH₂.

In a linker-drug compound according to the invention, preferably n is 0 or 1, X^{4a-b} and X^{4c-d} (when present) are O and R² and R⁴ (when present) are H.

R² and R³, and R⁴ when present, may be prodrug moieties selected from the group consisting of:
a pivaloyloxymethyl (POM) and isopropyloxycarbonyloxymethyl (POC) group,
a substituted or non-substituted (hetero)aryl group, and
a structure according to formula IV or V

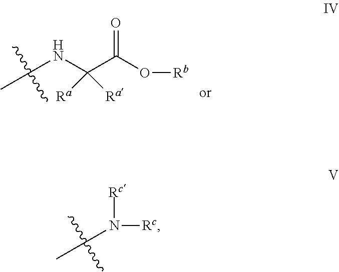

wherein;
R^{a} and R^{a′} are independently selected from H, an optionally substituted amino acid side chain and a non-polar side chain comprising an optionally substituted C₁₋₁₄ alkyl chain,
R^{b} is H, benzyl or a substituted or non-substituted (C₁₋₈)alkyl,
R^{c} and R^{c′} are independently selected from H and an, optionally substituted, C₁-C₆ alkyl, C₃-C₆ cycloalkyl, aryl or heteroaryl.

When R², R³, and R⁴ when present, are prodrug moieties, R² R³ and R⁴ may be independently selected from a POM- and POC-group.

When R² and R³ are prodrug moieties and n is 0, R² may be a substituted or non-substituted 5 or 6 membered (hetero)aryl group and R³ may be a structure according to formula IV or V, or vice versa.

The linking moiety (L) preferably is a cleavable linking moiety.

The linking moiety (L) may comprise a structure according to formula VI or VII

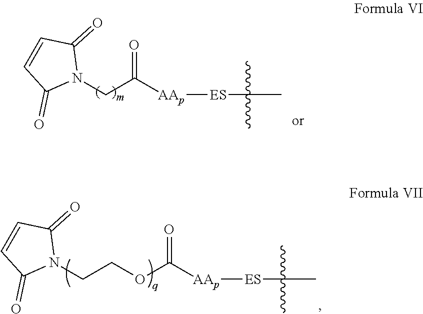

wherein
   m is an integer ranging from 1 to 10, preferably 5;
   AA is an amino acid, preferably a natural amino acid; and
   p is 0, 1, 2, 3, or 4;
   q is an integer ranging from 1 to 12, preferably 2;
   ES is either absent or an elongation spacer selected from

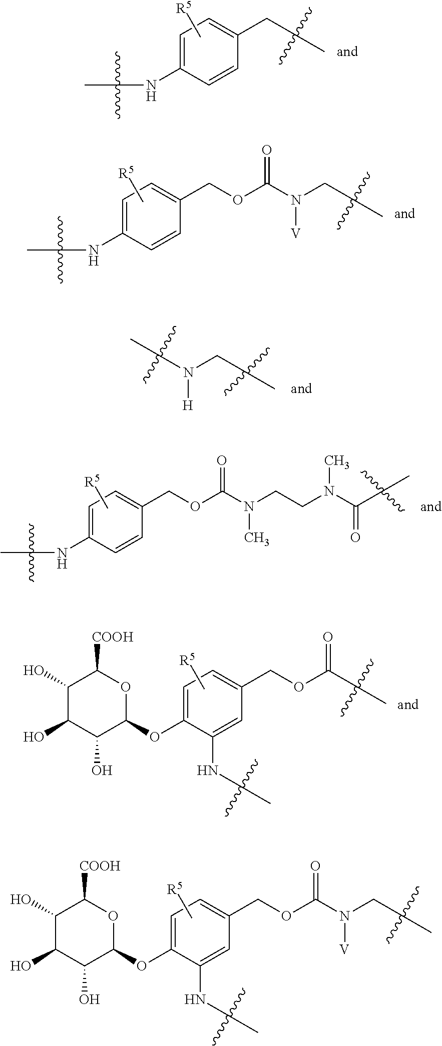

wherein
R⁵ is H, halogen, CF₃, C₁₋₄ alkyl, C₂₋₄ alkenyl, C₂₋₄ alkynyl, C₁₋₄ alkoxyl, or C₁₋₄ alkylthio, preferably H, F, CH₃, CF₃, more preferably H or F, and wherein V is H, ethyl, —(CH₂CH₂O)ₚ—OMe, CH₂CH₂SO₂Me or CH₂CH₂N(Me)₂, and wherein p is an integer ranging from 1 to 12.

Linker drug compounds according to the invention may be used in the manufacture of a conjugate. Further provided are conjugates comprising a targeting moiety, preferably a tumor-targeting antibody or antigen binding fragment thereof, covalently linked to a linker drug compound according to the invention. In such conjugates the linking moiety preferably comprises a cleavable linker. Conjugates according to the invention may be used as a medicament. Further provided is a pharmaceutical composition comprising a conjugate according to the invention and one or more pharmaceutical excipients.

Such conjugates can be used to activate gammadelta T-cells, for example, in the treatment of diseases such as cancer, infection, or autoimmune disease. Conjugates according to the present invention can be used either alone, or in combination with other therapeutic agents.

Preferably conjugates according to the invention are immunoconjugates comprising a tumor-targeting antibody or an antigen binding fragment thereof as targeting moiety. Such immunoconjugates according to the invention, comprising tumor-targeting antibodies as targeting moiety, can be used to specifically deliver phosphoantigens to localized tumor cells, where they may be internalized into the tumor cell after binding, of the antibody or antigen binding fragment thereof, to its tumor specific or tumor associated antigen (TAA).

### BRIEF DESCRIPTION OF THE DRAWINGS

FIG. 1 (A-C): CD107a (A, C) and IFNγ (B) production by gated Vdelta2 gammadelta T-cells (A, B) or NK-cells (C) after co-culture of PBMCs with a concentration range of pAg ADCs pretreated Raji cells. Levels of activation are indicated by the proportion of immune cell subsets that are CD107a- or IFNγ-positive. Measurements for each compound were performed in 2 independent experiments with one donor per experiment.

FIG. 2 (A-B): CD107a (A) and IFNγ (B) production by gated Vdelta2 gammadelta T-cells after co-culture of PBMCs with a concentration range of pAg ADCs pretreated Raji cells. Levels of activation are indicated by the proportion of immune cell subsets that are CD107a- or IFNγ-positive.

FIG. 3 Binding of pAg ADCs and rituximab to Raji cells, revealed using a fluorochrome-labeled goat anti-Human antibody. Results show the median fluorescence intensity (MFI) average value+/−standard deviation from two independent experiments.

FIG. 4 (A-C): CD107a (A, C) and IFNγ (B) production by gated Vdelta2 gammadelta T-cells (A, B) or NK-cells (C) after co-culture of PBMCs with a concentration range of pAg ADCs or rituximab pretreated Raji cells. Levels of activation are indicated by the proportion of immune cell subsets that are CD107a- or IFNγ-positive. Measurements for each compound were performed in two independent experiments with one donor per experiment.

### DETAILED DESCRIPTION OF THE PRESENT INVENTION

With the present invention linker drug compounds and conjugates are provided, comprising a phosphoantigen (pAg) moiety.

### Linker-Drug Compounds

The present invention provides linker-drug compounds, with the general structure reflected in formula (I)

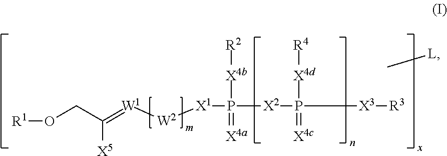

wherein L represents a linking moiety,
W¹ is N, CH or CF, preferably CH;
W² is CH₂, CHF, CF₂ or O;
X¹ is O, S, NH, CH₂, CHF or CF₂;
X² is O, CH₂, CHF or CF₂;
X³ is absent or O or NH;
each of X^{4a-d} is independently selected from O and S;
X⁵ is
   H, halogen (F, Cl, Br, I) or nitril (CN) or
   ethenyl, ethynyl, ethyl, optionally substituted with one or more of the following groups: F, CH₃, CH₂F, CHF₂, CF₃, or
   C₃-C₄ cycloalkyl, C₃-C₄ cycloalkenyl, allyl, propynyl, O—CH₃, S—CH₃, optionally substituted with one or more fluorine substituents, or
   CHR¹OR², CHR¹SR², CHO, CO₂R¹, CONR¹R², where R¹ and R² are independently selected from H, CH₃, CH₂F, CHF₂, CF₃, CH₂CH₃;
x is an integer ranging from 1-5;
m is 1, 2 or 3;
n is 0, 1 or 2;
R¹ is H or a connection to the linking moiety (L) or a prodrug moiety;
R² is H or a connection to the linking moiety (L) or Cat+ or a prodrug moiety;
R³ is H or a connection to the linking moiety (L) or Cat+ or a prodrug moiety;
R⁴ is H or a connection to the linking moiety (L) or Cat+ or a prodrug moiety;
or, when n is 0, R³ and R² are connected by a C₁₋₆ (hetero)alkyl group or;
when n is 1 or 2, R³ and R⁴ are connected by a C₁₋₆ (hetero)alkyl group.

When X⁵ is ethenyl, ethynyl or ethyl, X⁵ may be substituted with one or more of the following groups: F, CH₃, CH₂F, CHF₂, CF₃. Said ethenyl, ethynyl or ethyl may thus be substituted with one or more fluorine substituents. When X⁵ is ethenyl, ethynyl or ethyl, substituted with one or more (fluorinated) methyl groups (CH₃, CH₂F, CHF₂ or, CF₃), the number of (fluorinated) methyl groups preferably is 1. This includes compounds where X⁵ is (iso) propyl.

C₃-C₄ cycloalkyl includes a cyclopropyl, methylcyclopropyl and cyclobutyl. C₃-C₄ cycloalkenyl includes cyclopropenyl, methylcyclopropenyl, and cyclobutenyl. When X⁵ is C₃-C₄ cycloalkyl, C₃-C₄ cycloalkenyl, allyl, propynyl, O—CH₃, S—CH₃, X⁵ may also be substituted with one or more fluorine substituents. The number of fluorine substituents on X⁵ depends on the structure of X⁵. X⁵ can carry multiple fluorine substituents, either on separate- or on the same C-atoms, or both.

Preferably X⁵ is H, C₁, F, I or Br, most preferably Cl or Br.

The linker drug compounds according to the invention are modifications of linker drug compounds disclosed in co-pending patent application number WO2023/275025, filed in the name of Byondis B. V, and differ therefrom in the definition of X⁵.

Linker-drug compounds according to the invention comprise at least one phosphoantigen moiety (pAg or “drug”), represented by the structural formula between the outer brackets in Formula I, attached to a linking moiety (L or “linker”). The number of pAg moieties per linker is represented by “x”. When x is larger than 1, there are multiple pAg moieties connected to one (branched) linker moiety.

Preferably n is 0 or 1, most preferably 0. When n is 1 or 2, X² preferably is O. Linker-drug compounds with phosphoantigen moieties wherein n is 1 and X² is CH₂ or where n is 1 and X² is O are likewise part of the present invention. In this case each of X^{4a-d} preferably is O. In such linker drug compounds R³ or R¹ may represent a connection to the linking moiety, preferably R³ represent a connection to the linking moiety.

When n is 2, X² will appear twice in formula I, and can be referred to as X^{2a} and X²b which can be independently selected from O, CH₂, CHF and CF₂. When n is 2, R⁴ will also appear twice, and can be referred to as R^{4a} and R^{4b}, which can be independently selected from H, a connection to the linking moiety (L), Cat+ and a prodrug moiety. The same goes for X^{4c} and X^{4d}, when n is 2. Both appear twice (X^{4c}, X^{4ci}, X^{4d} and X^{4di}), and may be independently selected from O and S.

When m is 2 or 3, W² will appear multiple times in formula I and each W² can independently be selected from CH₂, CHF, CF₂ or O. Preferably W² is CH₂. In a preferred embodiment m is 1, and most preferably, when m is 1, W² is CH₂.

Cat+ represents an (organic or mineral) cation, including a proton.

X¹ preferably is CH₂, O or S, most preferably CH₂.

Each of X^{4a-d} (when present) preferably are O. Part of the present invention are compounds wherein n is 1 or 0 and wherein X^{4a-b} and X^{4c-d} (when present) are O and wherein R² and R⁴ (when present) preferably are H.

Preferably n is 0, and X⁴a as well as X^{4b} are O and R² preferably is H.

Preferably W¹ is CH or CF, most preferably CH. R¹ preferably is H or a connection to the linking moiety (L), most preferably H.

Preferably, W¹ is CH, X¹ is CH₂ and R¹ is H, resulting in a linker drug molecule carrying a pAg moiety with an allylic alcohol group. Preferably, when W¹ is CH, X¹ is CH₂ and R¹ is H, W² is CH₂ and m is 1.

Preferably X³ is O, R³ is a connection to a cleavable linking moiety, W¹ is CH, R¹ is H, W² is CH₂ and m is 1, and X¹ is CH₂.

In such compounds R², R³ and/or R⁴ can be a prodrug moiety, either alone or in combination with X^{4b}, X^{4d}, and/or X³ (when X³ is present) respectively (the prodrug moiety being —X^{4b}—R², —X^{4d}—R⁴ and/or —X³—R³).

Preferably, W¹ is CH, W² is CH₂, X^{4a-d} are O, R² and R⁴ are H, and m is 1.

In a preferred embodiment, W¹ is CH, W² is CH₂, n is 0, X^{4a-b} are O and m is 1.

In formula I, x represents the number of phosphoantigen moieties (pAg) per linking moiety (L), wherein the structure between the brackets thus is a structural representation of phosphoantigen moieties preferably used in linker-drug compounds according to the invention. X can be an integer in the range from 1-5 (each linking moiety carries one to 5 pAgs). Preferably, a linking moiety carries 1 or 2 pAg moieties. In most instances it may suffice for each linking moiety to carry 1 pAg.

The connection to the linking moiety can be (part of) R¹, or, in the alternative, the linking moiety may be (connected to) R², R³ or R⁴. Preferably either R¹ or R³ is a connection to the linking moiety, more preferably R³. When the linking moiety is connected at R³, X³, preferably, is O. When R³ is a connection to the linker moiety, preferably X³ is O and R¹ is preferably H.

When the linking moiety is attached at the R² or R⁴ position, X^{4b} or X^{4d} respectively, preferably is O.

Preferred linker drug compounds are those wherein X³ is O and R³ is a connection to a cleavable linking moiety, wherein preferably W¹ is CH, R¹ is H, W² is CH₂ and m is 1, and X¹ is CH₂. In such compounds n is preferably 0.

With “a connection to the linking moiety” the location in the molecule where the linker is connected to the phosphoantigen moiety is meant. “Connection” doesn't necessarily mean that R¹, R², R³ or R⁴ (depending on where the linker is connected) represent actual (remaining) structural elements of the linker-drug compound between the linker and the remainder of the phosphoantigen moiety. For example, depending on the linker chemistry used, when R¹ represents a connection to the linking moiety, this also includes the situation where the linker is directly connected to the oxygen atom of the phosphoantigen moiety in the linker-drug molecule. In the alternative R¹ is a connection to the linking moiety (L). In such instances where R¹ is a connection to the linking moiety (L), preferably W¹ is CH, W² is CH₂, m is 1 and X¹ is CH₂. When such a linker-drug molecule is incorporated into a conjugate according to the invention, cleavage of the linker after administration may result in the (re-)formation of an allyl alcohol group (R¹ is H, in the actual functionally active phosphoantigen moiety released from the conjugate). R¹ can also be a prodrug moiety. Suitable alcohol prodrug moieties are known in the art. For example, an alcohol can be masked by an ester based prodrug group. Creation of the active alcohol relies on the hydrolysis of the ester bond by (cellular) esterases, resulting in the metabolic regeneration of an alcohol (drug) and a carboxylic acid (leaving group).

R², R³, and R⁴ can each independently be H or a connection to the linking moiety (L) or Cat+ or a prodrug moiety. In a preferred embodiment, compounds according to the invention are monophosphonates (n is 0) and R⁴ is thus absent.

Cat+ represents an (organic or mineral) cation, including a proton (and may be exchanged in a formulation buffer or plasma). When R², R³ and/or R⁴ are Cat+, Cat+ may be identical or different. Preferably, when R², R³ and/or R⁴ are Cat+, X^{4b} and X^{4d} (when present, i.e., n is not 0), and/or X³ are O, resulting in O-Cat+.

In another embodiment of the invention, where n is 0, R³ and R² are connected by a C₁₋₆ (hetero)alkyl group. In this case R³ and R² together form a substituted or non-substituted 5-8 membered ring. In such an embodiment the linking moiety is preferably connected at the R¹ position. In an alternative embodiment where n is not 0, R³ and R⁴ may be connected in a similar way by a C₁₋₆ (hetero)alkyl group.

R², R³, and/or R⁴ can also be a prodrug moiety, either alone or in combination with X^{4b}, X^{4d}, and/or X³ (when X³ is present) respectively (the prodrug moiety being —X^{4b}—R², —X^{4d}—R⁴ and/or —X³—R³).

A “prodrug moiety” can be a group that can either be non-enzymatically or enzymatically cleaved (releasing the active compound). A “prodrug moiety” may induce release of a second prodrug moiety on another position in the molecule, after a conjugate according to the invention is administered to a subject. Preferably a phosphoantigen moiety in the form of a prodrug is converted to a functionally active phosphoantigen inside the target cell (e.g., a tumor cell), for example by enzymatic removal of prodrug moieties.

Examples of prodrug technologies known in the art include the use of pivaloyloxymethyl (POM) or isopropyloxycarbonyloxymethyl (POC) groups. In a preferred embodiment, at least R² is and R³ are independently selected from a POM- or POC-group (for example, when n is 0). When n is 1 or 2, R⁴ may be a POM or POC group as well.

Phosphoantigen prodrugs of this kind are described, for example, in WO2019/182904.

In the alternative a combination of leaving groups can be used; An example of such prodrug technology is the “ProTide” technology, developed for intracellular delivery of monophosphates and monophosphonates. The hydroxyls of the monophosphate or monophosphonate groups in a ProTide prodrug are masked (or replaced) by an aromatic group and an amino acid ester moiety, which are enzymatically cleaved-off inside cells to release the free monophosphate and monophosphonate (Mehellou et al. 2018, Journal of Medicinal Chemistry, 61 (6), 2211-2226).

Linker-drug compounds and conjugates according to the invention, wherein the phosphoantigen moiety is a monophosphate or monophosphonate, and wherein R² and R³ are a combination of “ProTide” leaving groups are therefore also part of the present invention. In such cases wherein the phosphoantigen moiety is a ProTide prodrug of a phosphoantigen, either R² is an aromatic moiety and R³ is an amino acid ester moiety or vice versa. In preferred embodiment of the invention, when n is 0, either R² or R³ may be a substituted or non-substituted (hetero)aryl group, while the other (either R³ or R²) may be selected from a structure according to formula IV and V

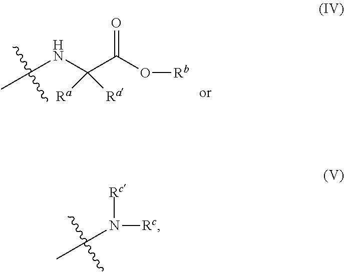

wherein;
R^{a} and R^{a′} are independently selected from H, an optionally substituted amino acid side chain and a non-polar side chain comprising an optionally substituted C₁₋₁₄ alkyl chain,
R^{b} is H, benzyl or a substituted or non-substituted (C₁₋₈)-alkyl,
R^{e} and R^{e′} are independently selected from H, or an optionally substituted (C₁₋₆)-alkyl, (C₃₋₆) cycloalkyl, aryl or heteroaryl. R^{e} and R^{e′} may also, together with the nitrogen they are bound to, form an, optionally substituted ring, such as an aziridino-, azetidino-, morpholino-, piperazino-, pyrrolidino- or piperidino-ring.

Optional substituents on R^{e} and/or R^{e′} are a carboxylic acid bioisostere, amino, tetrazole, sulfonate, hydroxyl, halo or alkyl.

When R^{b} is a substituted alkyl, substituents may be one or more groups independently selected from the group consisting of hydroxy, amino, halo, nitro, cyano, carboxy, NRₓR_{y}, (C₁₋₆)alkoxy, (C₁₋₆)alkanoyl, (C₁₋₆)alkoxycarbonyl, (C₁₋₆)alkylthio, and (C₂₋₆)alkanoyloxy, wherein each Rₓ and R^{y} is independently selected from the group consisting of H, (C₁-C₆)alkyl, (C₃₋₆) cycloalkyl, and (C₃₋₆) cycloalkyl (C₁₋₆)alkyl. In the alternative Rₓ and R_{y} together with the nitrogen to which they are attached form a aziridino, azetidino, morpholino, piperazino, pyrrolidino or piperidino group.

### Linking Moiety

A linking moiety (or “linker”) for use in a conjugate or linker-drug compound according to the invention preferably is a synthetic linker. The structure of a linker is such that the linker can be easily chemically attached to a small effector molecule (the phosphoantigen moiety), and so that the resulting linker-drug compound can be easily conjugated to a further substance such as for example a polypeptide (e.g. an antibody). The choice of linker can influence the stability of such eventual conjugates when in circulation, and it can influence in what manner the small molecule effector compound (a phosphoantigen) is released, if it is released. Suitable linkers are for example described in Ducry et a.l, 2010, Bioconjugate Chem., 21, 5-13, King and Wagner, 2014, Bioconjugate Chem., 25, 825-839; Gordon et al., 2015, Bioconjugate Chem., 26, 2198-2215; Tsuchikama and An, 2018, Protein & Cell, 9, 33-46 DOI: 10.1007/s13238-016-0323-0; Polakis, 2016, Pharmacological Reviews, 68 (1), 3-19, DOI: 10.1124/pr.114.009373; Bargh et al., 2019, Chem. Soc. Rev., 48, 4361-4374, DOI: 10.1039/c8cs00676h; WO 02/083180, WO2004/043493, WO2010/062171, WO2011/133039, WO2015/177360, and in WO2018/069375. Linkers may be cleavable or non-cleavable as described in e.g., van Delft, F and Lambert, J. M., 2021, Chemical Linkers in Antibody-Drug Conjugates (ADCs), 1st Ed. Royal Society of Chemistry, ISBN-10:1839162635. Another way of coupling linker-drugs to antibodies is by making use transpeptidases such as bacterial sortases or plant asparaginyl endopeptidases, enabling the site-specific installation of chemical moieties attached to an appropriate synthetic peptide. Sortase A (Sort-A) recognizes a C-terminal peptide sequence (LPXTG) and creates a bond between the threonine within this sequence and a glycine provided on the N terminus of the conjugation partner, e.g. a glycine tagged payload for an ADC (Combs et al., 2015, the AAPS Journal, Vol. 17, No. 2, 339-351, DOI: 10.1208/s12248-014-9710-8). Antibody drug conjugation can also be achieved through site-specific glycoengineering, for example by using endo-β-N-acetylglucosaminidase (ENGases) and monosaccharyl transferase mutants (Manabe et al., 2021, Chem Rec, (11), 3005-3014, doi: 10.1002/tcr.202100054; Wang et al., 2019, Annu Rev Biochem, 20; 88, 433-459, doi: 10.1146/annurev-biochem-062917-012911).

The use of cleavable linkers in conjugates according to the invention is preferred. Cleavable linkers comprise moieties that can be cleaved, e.g., when exposed to lysosomal proteases or to an environment having an acidic pH or a higher reducing potential. Suitable cleavable linkers are known in the art and comprise e.g., a mono-, di-, tri- or tetrapeptide, i.e., a single-, two, three or four amino acid residues. Additionally, the cleavable linker may comprise a selfimmolative moiety such as an ω-amino aminocarbonyl cyclization spacer, see Saari et al, 1990, J. Med. Chem., 33, 97-101, or a —NH—CH₂—O— moiety.

Other cleavable linkers known in the art are beta-glucuronide linkers, such as those disclosed in, for example, Jeffrey et al., 2006, Bioconjugate Chem. 2006, 17, 831-840. Cleavage of the linker makes the immunomodulating effector moiety (phosphoantigen or “pAg” moiety) in a conjugate according to the invention available to the surrounding environment. Non-cleavable linkers can still effectively release (an active derivative of) the phosphoantigen moiety from the immunoconjugate according to the invention, for example when a conjugated polypeptide (antibody) is degraded in the lysosome. Non-cleavable linkers include e.g., succinimidyl-4-(N-maleimidomethyl(cyclohexane)-1-carboxylate and maleimidocaproic acid and analogs thereof.

To be able to conjugate a linking moiety or linker-drug compound to a polypeptide, such as an antibody, the side of the linking moiety that will be (covalently) bonded to the antibody, typically contains a functional group that can react with an amino acid residue of the antibody, under relatively mild conditions. This functional group is referred to herein as a reactive moiety (RM). Examples of reactive moieties include, but are not limited to, carbamoyl halide, acyl halide, active ester, anhydride, alpha-halo acetyl, alpha-halo acetamide, maleimide, isocyanate, isothiocyanate, disulfide, thiol, hydrazine, hydrazide, sulfonyl chloride, aldehyde, methyl ketone, vinyl sulfone, halo methyl, methyl sulfonate, cyclooctyn and trans-cyclooctene (TCO). Such amino acid residue with which the functional group reacts may be a natural or non-natural amino acid residue, or a (non-) natural glycan (Manabe et al., Wang et al., vide supra). The term “non-natural amino acid” as used herein is intended to represent a (synthetically) modified amino acid or the D-stereoisomer of a naturally occurring amino acid. Preferably, the amino acid residue with which the functional group reacts is a natural amino acid.

Linking moieties (L) for use in conjugates or linker-drug compounds according to the present invention may comprises a structure according to formula VI or VII

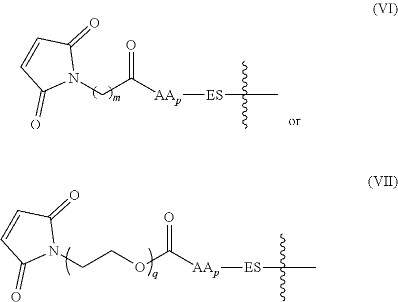

wherein m is an integer ranging from 1 to 10, preferably 5; A is an amino acid, preferably a natural amino acid and p is 0, 1, 2, 3, or 4. When p is more than 1, the aminoacids may be the same or different.

Suitable amino-acid combinations are known in the art and include amino acids selected from the group consisting of alanine, glycine, lysine, phenylalanine, valine, and citrulline. Preferably p is 2. When p is 2, AA₂ may be, for example, phenylalanyllysine, valylalanine, valylcitrulline or valyllysine. When p is 2, AA₂ preferably is valylalanine or valylcitrulline. When p is 3, AA₃ may be, for example, alanylphenylalanyllysine, when p is 4, AA₄ may be, for example, glycylglycylphenylalanylglycine.

“q” is an integer ranging from 1 to 12, preferably 2; ES is either absent or an elongation spacer selected from

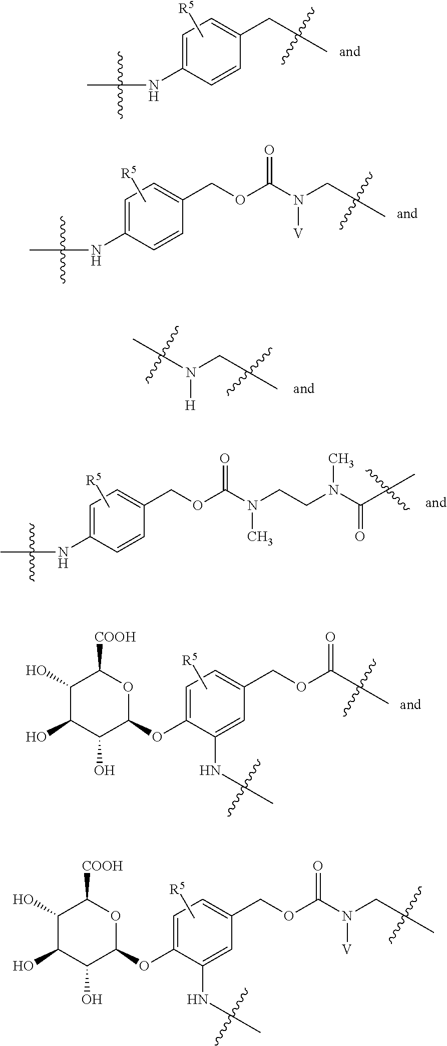

wherein R⁵ is H, halogen, CF₃, C₁₋₄ alkyl, C₂₋₄ alkenyl, C₂₋₄ alkynyl, C₁₋₄ alkoxyl, or C₁₋₄ alkylthio, preferably H, F, CH₃ or CF₃, more preferably H or F; and V is H, ethyl, —(CH₂CH₂O)ₚ—OMe, CH₂CH₂SO₂Me or CH₂CH₂N(Me)₂, wherein p is an integer ranging from 1 to 12.
   When ES is present, ES is preferably selected from

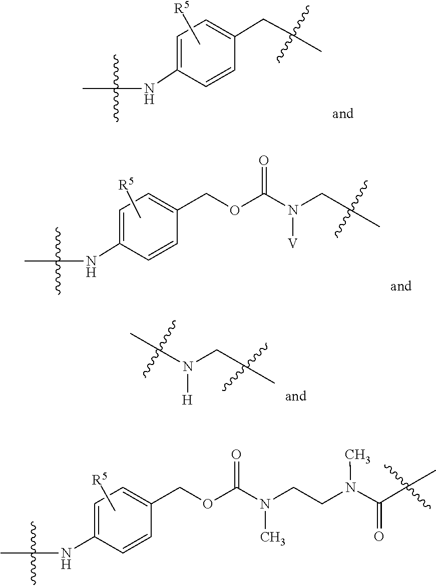

wherein R⁵ is H, halogen, CF₃, C₁₋₄ alkyl, C₂₋₄ alkenyl, C₂₋₄ alkynyl, C₁₋₄ alkoxyl, 10 or C₁₋₄ alkylthio, preferably H, F, CH₃ or CF₃, more preferably H or F; and V is H, ethyl, —(CH₂CH₂O)ₚ—OMe, CH₂CH₂SO₂Me or CH₂CH₂N(Me)₂, wherein p is an integer ranging from 1 to 12.

AAₚ, is preferably absent (meaning p is 0), when ES is selected from

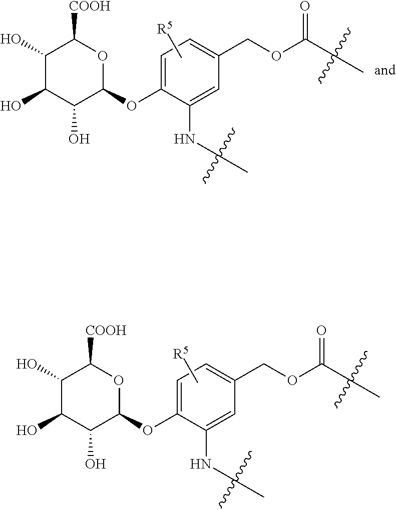

wherein R⁵ is H, halogen, CF₃, C₁₋₄ alkyl, C₂₋₄ alkenyl, C₂₋₄ alkynyl, C₁₋₄ alkoxyl, or C₁₋₄ alkylthio, preferably H, F, CH₃ or CF₃, more preferably H or F; and V is H, ethyl, —(CH₂CH₂O)ₚ—OMe, CH₂CH₂SO₂Me or CH₂CH₂N(Me)₂, wherein p is an integer ranging from 1 to 12.

Linking moieties can also be branched, which results in one linking moiety being able to carry multiple phosphoantigen moieties. Examples of branched linking moieties are:

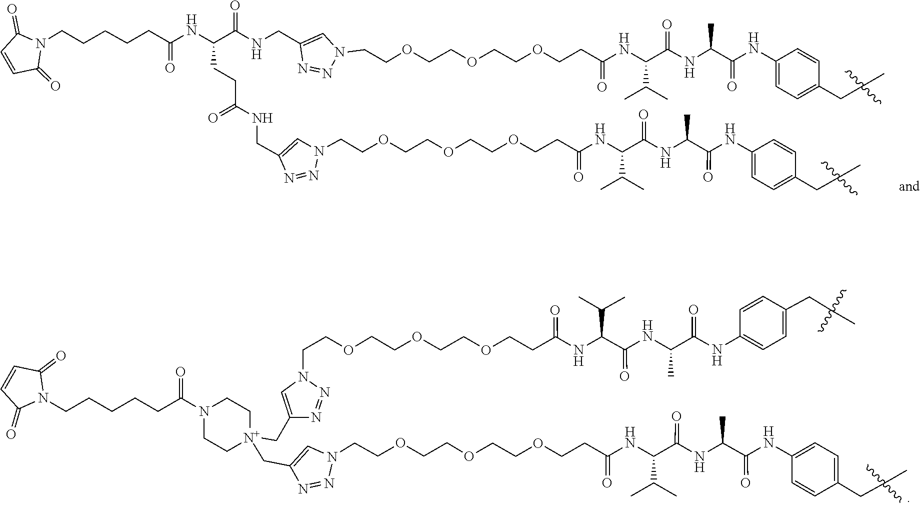 [SMILES: *Cc1ccc(NC(=O)[C@H](C)NC(=O)[C@@H](NC(=O)CCOCCOCCOCCn2cc(CNC(=O)CC[C@H](NC(=O)CCCCCN3C(=O)C=CC3=O)C(=O)NCc3cn(CCOCCOCCOCCC(=O)N[C@H](C(=O)N[C@@H](C)C(=O)Nc4ccc(C*)cc4)C(C)C)nn3)nn2)C(C)C)cc1 ; *Cc1ccc(NC(=O)[C@H](C)NC(=O)[C@@H](NC(=O)CCOCCOCCOCCn2cc(C[N+]3(Cc4cn(CCOCCOCCOCCC(=O)N[C@H](C(=O)N[C@@H](C)C(=O)Nc5ccc(C*)cc5)C(C)C)nn4)CCN(C(=O)CCCCCN4C(=O)C=CC4=O)CC3)nn2)C(C)C)cc1]

These branched linker moieties can be used to create conjugates with a relatively high pAg to targeting moiety ratio (“DAR”). Using such branched linkers, conjugates with a DAR of 16 and even 20 or higher can be synthesized. Antibody based conjugates according to the invention may only need a DAR of about 2. However, for antibodies to tumor specific targets that are known to be expressed at a relatively low level on target tumor cells, conjugates with a high pAg to targeting moiety ratio may be preferred. Linker-drug compounds for use in a linker-drug compound according to the invention can, for example, contain any linking moiety selected from:

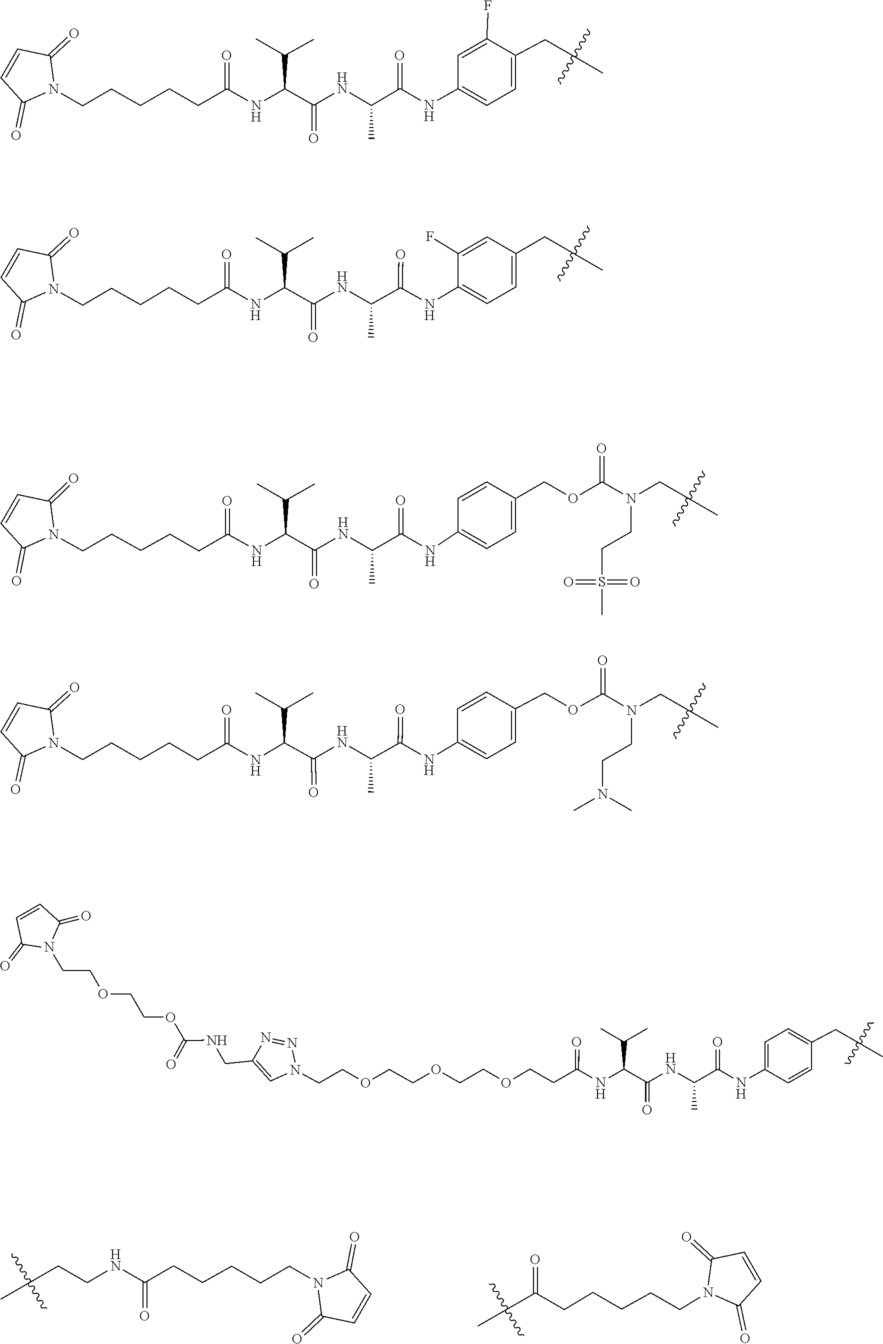

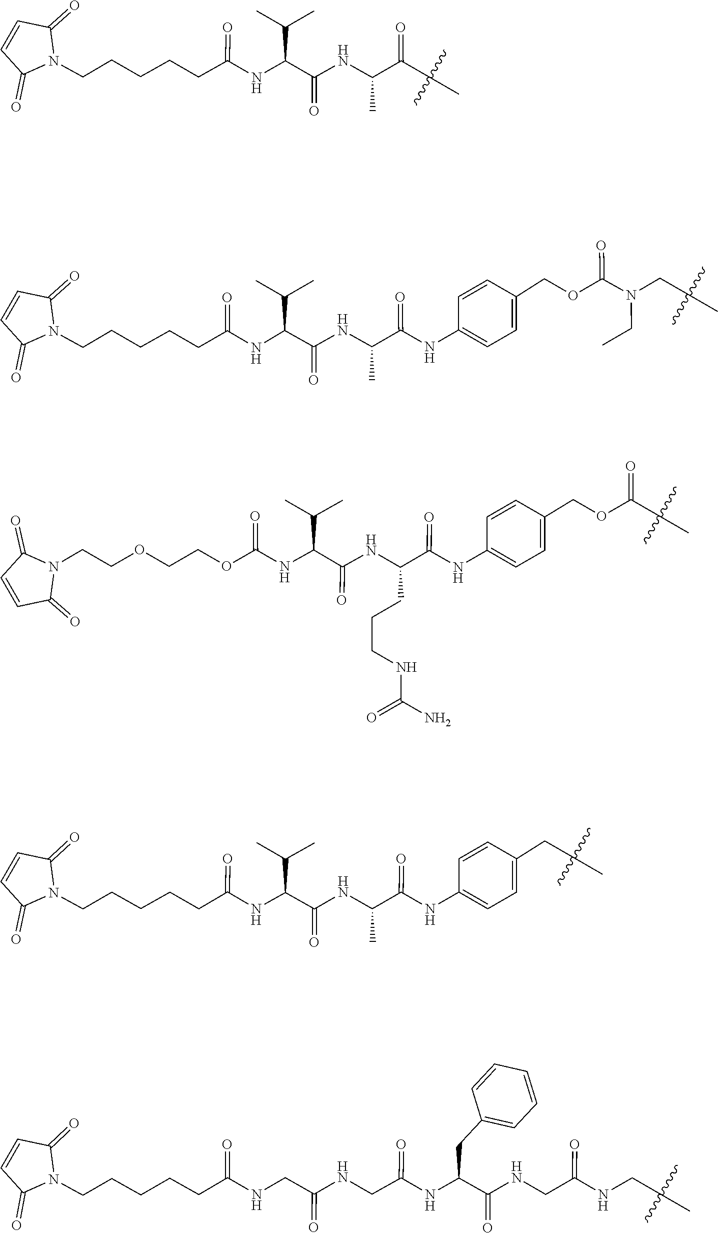 [SMILES: *C(=O)[C@H](C)NC(=O)[C@@H](NC(=O)CCCCCN1C(=O)C=CC1=O)C(C)C ; *CN(CC)C(=O)OCc1ccc(NC(=O)[C@H](C)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)OCCOCCN2C(=O)C=CC2=O)C(C)C)cc1 ; *Cc1ccc(NC(=O)[C@H](C)NC(=O)[C@@H](NC(=O)CCCCCN2C(=O)C=CC2=O)C(C)C)cc1 ; *CNC(=O)CNC(=O)[C@H](Cc1ccccc1)NC(=O)CNC(=O)CNC(=O)CCCCCN1C(=O)C=CC1=O]

Linker moieties (L) may be conjugated to pAg moieties resulting in linker drug compounds according to the invention with the general formula depicted in formula I.

Linker-drug compounds according to the invention can be conjugated to a targeting moiety, to create a conjugate according to the invention. Preferred conjugates according to the invention comprise a tumor targeting antibody, or antigen binding fragment thereof, conjugated to a linker drug compound according to the invention.

In a specific embodiment of the invention, the phosphoantigen moiety, as part of a conjugate according to the invention, is a monophosphonate prodrug, wherein the negatively charged non-binding oxygen atoms of the phosphonate group are protected by prodrug moieties such as a combination of ProTide moieties (a (hetero)aryl group and an amino ester radical) or one or more POM or POC, while a cleavable linking moiety may be attached to an isoprene unit of the phosphoantigen molecule, which will be converted to an allylic alcohol, found in phosphoantigens such as HMBPP, once the linker is cleaved.

The synthesis of examples of linker-drug molecules according to the invention is further exemplified in the Examples. Examples of preferred linker drug compounds according to the invention are depicted in table 1, in the “Examples” section of this patent application.

It is to be understood that a linker-drug compound comprising at least one phosphoantigen moiety covalently bound to a linking moiety according to the invention, when comprised in a conjugate according to the invention, may lack or gain certain atoms or groups of atoms, for example, it may lack a hydrogen atom as compared to the same linker-drug compound according to the invention when not comprised in a conjugate. This can be for example because the linker-drug compound according to the invention is conjugated to a polypeptide via, for example, esterification to a hydroxyl moiety.

For example, when the targeting moiety is an antibody or antigen binding fragment thereof, one or more linker-drug compounds according to the invention can be conjugated to the targeting antibody, thus creating a conjugate according to the invention.

### Conjugates

The present invention provides a conjugate, comprising a targeting moiety (Tm) covalently linked to one or more linker drug compounds according to the invention.

Conjugates according to the invention comprise a targeting moiety that specifically binds to a target cell. Preferably the targeting moiety is a tumor-targeting antibody or antigen binding fragment thereof. The targeting moiety serves as a delivery vehicle; it delivers, to a target cell, the pAg moiety covalently linked to the targeting moiety. The pAg moiety may be coupled directly to, for example, an amino acid side chain in a (polypeptide) targeting moiety. Preferably, however, the pAg is conjugated to a targeting moiety, via a linking moiety.

Preferred conjugates according to the present invention can be represented by the general formula II

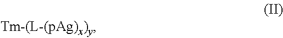

wherein Tm represents a targeting moiety, preferably an antibody or an antigen binding fragment thereof, L represents a linking moiety and corresponds to the “L” moiety in Formula I, and pAg represents a phosphoantigen moiety and corresponds to the structural formula between the outer brackets in Formula I, x represents the number of phosphoantigen moieties per linking moiety (as in Formula I), and has a value ranging from 1-5 and y represents the average number of L-(pAg) x, per Tm (linker drug moieties per targeting moiety) and is an integer ranging from 1-10, preferably 1-8. The number of pAg moieties per conjugate (pAg to Tm ratio) in formula II is x multiplied by y. The average pAg-to-Tm ratio can be in the range from 1 to 16, or even 20, or higher. The ratio of pAg units per targeting moiety can be varied, for example, based on structural or functional characteristics of either the phosphoantigen moiety or the targeting moiety. In practice, the number of pAg per targeting moiety in the range of 2-8 or 2-6, or even as low as 2 may provide a sufficient therapeutic effect. Preferably, a linking moiety carries 1 or 2 pAg moieties. In most instances it may suffice for each linking moiety to carry 1 pAg. Preferably the target pAg to Tm ratio is 2 (x is 1 and y is 2).

Linker moieties preferably are cleavable linker moieties. In conjugates according to the invention straight or branched linker moieties may be used. When multiple phosphoantigen moieties are linked to one targeting moiety, each phosphoantigen moiety may be covalently coupled to the targeting moiety by a separate linking moiety. In practice, when the targeting moiety is an antibody, and coupling occurs to reduced interchain disulfides, there may be as many as 8 separate linking moieties (linker drug moieties) attached to one targeting moiety, resulting in 8 phosphoantigen moieties per target moiety when each phosphoantigen moiety is carried by its own linking moiety. In the alternative branched linker moieties may carry 1-5 phosphoantigen moieties per linking moiety (x is 1, 2, 3, 4 or 5).

Especially when a higher pAg to Tm ratio is desired, or when only a limited number of binding places are available on a targeting moiety, branched linkers are preferred. For example, branched linkers carrying 2 pAgs (x is 2) can be used to increase the number of phosphoantigen moieties per targeting moiety to a higher value. By using such linking moieties, e.g. 16 phosphoantigen moieties can be bound to a targeting moiety using only 8 linking moieties. Antibodies can be modified to introduce additional cysteines, in addition to the number of cysteines, in the antibody amino acid sequence, that form disulfide bonds and can be reduced and conjugated to a linker-drug molecule. For example, additional cysteines can be introduced at positions such as the 41C position, as disclosed in WO2015177360. For antibodies containing as many as 10 cysteines available for conjugation to which linking moieties can be bound, a DAR of 20 (x is 2, y is 10) or higher can even be reached, when branched linkers carrying two pAg moieties per linker (x is 2) are used. Under optimal conditions, all binding sites in a targeting moiety will be occupied by a linking moiety. In practice, a conjugate mixture may be produced wherein the exact number of phosphoantigen moieties per target moiety may vary somewhat, depending on the reaction conditions, and y values are average numbers.

Conjugates according to the invention may be used in combination with other pharmaceutically active compounds that may be simultaneously or sequentially administered to a subject in need thereof. Additionally, a targeting moiety may carry a combination of a linker drug compound according to the invention and a different payload. The advantage of such a “multiple payload” approach is that both actives will be targeted by the same targeting moiety. The ratio between the payloads of course has to be appropriately set by the (conjugation) reaction conditions and binding sites. Separate linker-drug compounds for each payload may, for example, be conjugated to different binding sites (e.g. different types of amino acids) on the targeting moiety and/or be conjugated by different conjugation methods and/or different linker chemistry to control the binding, distribution and drug to antibody ratio (DAR) of both payloads. Antibody drug conjugates (ADCs) carrying multiple cytotoxic payloads are known in the art. Conjugates according to the invention may combine a phosphoantigen moiety, for example, with a cytotoxic payload or with another immunomodulatory payload designed to enhance the overall desired therapeutic effect. Any non-specific binding to- and/or effects on non-target tissue of a phosphoantigen at non-target sites, is thus diminished.

As is well-known in the art, the drug load distribution in an ADC can be determined, for example, by using hydrophobic interaction chromatography (HIC) or reversed phase high-performance liquid chromatography (RP-HPLC). HIC is particularly suitable for determining the average DAR (pAg to Tm ratio in a conjugate according to the invention).

### Targeting Moiety

A targeting moiety specifically or preferably binds to a target cell and can be a targeting antibody, or an antigen binding fragment thereof, or another targeting moiety such as, for example, nucleic acids (aptamers) or (poly) peptides, which may be enzyme inhibitors, enzyme substrates, receptor ligands, and/or fusion proteins. Also small-molecule inhibitors can be used as targeting moieties (resulting in small molecule drug conjugates (SMDC's). The binding specificity (and affinity) of the targeting moiety for its target determine where, in the body, a conjugate according to the invention will exert its therapeutic effect.

Thus, by selecting an appropriate targeting moiety, it is ensured that a phosphoantigen moiety is delivered at the site where it has to exert its therapeutic effect.

Preferably the targeting moiety in a conjugate according to the invention is an antibody, or an antigen binding fragment thereof. In case the targeting moiety is an antibody, or an antigen binding fragment thereof, conjugates are commonly referred to as immunoconjugates, or antibody drug conjugates (ADC). Targeting antibodies are antibodies that recognize an antigen expressed by a target cell, such as a tumor associated antigen, with high specificity. The specificity of the antibody or fragment for its antigen allows for the specific delivery of an effector molecule (or “payload”) to the target cell, leaving healthy tissue largely unaffected. An effector molecule is covalently coupled to the antibody via a linker that ensures that the effector molecule stays connected to the antibody, at least until the antibody reaches the target cell, e.g. a cancer cell. Effector molecules exert their effect on or in (when the conjugate is internalized) the target cell when the antibody binds to its target. Effector molecules can be cytotoxic agents, radioisotopes, or immunomodulating moieties. In conjugates according to the invention the effector molecule is a phosphoantigen moiety.

### Antibody

The term “antibody” as used herein preferably refers to an antibody comprising two heavy chains and two light chains. Generally, the antibody or any antigen-binding fragment thereof, is one that has a therapeutic activity, but such independent efficacy is not necessarily required, as is known in the art of ADCs. The antibodies to be used in accordance with the invention may be of any isotype such as IgA, IgE, IgG, or IgM antibodies. Preferably, the antibody is an IgG antibody, more preferably an IgG1 or IgG2 antibody. The antibodies may be chimeric, humanized or human. Preferably, the antibodies are humanized or human. Even more preferably, the antibody is a humanized or human IgG antibody, more preferably a humanized or human IgG1 monoclonal antibody. The antibody may have k (kappa) or 2 (lambda) light chains, preferably K (kappa) light chains, i.e., a humanized or human IgG1-K antibody.

The term “antigen-binding fragment” as used herein includes a Fab, Fab′, F(ab′)₂, Fv, scFv or reduced IgG (rIgG) fragment, a single chain (sc) antibody, a single domain (sd) antibody, a diabody, or a minibody.

“Humanized” forms of non-human (e.g., rodent) antibodies are antibodies (e.g., non-human-human chimeric antibodies) that contain minimal sequences derived from the non-human antibody. Various methods for humanizing non-human antibodies are known in the art. For example, the antigen-binding complementarity determining regions (CDRs) in the variable regions (VRs) of the heavy chain (HC) and light chain (LC) are derived from antibodies from a non-human species, commonly mouse, rat or rabbit. These non-human CDRs may be combined with human framework regions (FRs, i.e., FR1, FR2, FR3 and FR4) of the variable regions of the HC and LC, in such a way that the functional properties of the antibodies, such as binding affinity and specificity, are at least partially retained. Selected amino acids in the human FRs may be exchanged for the corresponding original non-human species amino acids to further refine antibody performance, such as to improve binding affinity, while retaining low immunogenicity. The thus humanized variable regions are typically combined with human constant regions. Exemplary methods for humanization of non-human antibodies are the method of Winter and co-workers (Jones et al, 1986, Nature, 321, 522-525; Riechmann et al, 1988, Nature, 332, 323-327; Verhoeyen et al, 1988, Science 239, 1534-1536). Alternatively, non-human antibodies can be humanized by modifying their amino acid sequence to increase similarity to antibody variants produced naturally in humans. For example, selected amino acids of the original non-human species FRs are exchanged for their corresponding human amino acids to reduce immunogenicity, while retaining the antibody's binding affinity. For further details, see Jones et al, vide supra, Riechmann et al., vide supra and Presta, 1992, Curr. Op. Struct. Biol. 2, 593-596. See also the following review articles and references cited therein: Vaswani and Hamilton, 1998, Ann. Allergy, Asthma and Immunol., 1, 105-115; Harris, 1995, Biochem. Soc. Transactions, 23, 1035-1038; and Hurle and Gross, 1994, Curr. Op. Biotech., 5, 428-433.

The CDRs may be determined using the approach of Kabat (in Kabat, E. A. et al, (1991), Sequences of Proteins of Immunological Interest, 5th Ed. Public Health Service, National Institutes of Health, Bethesda, MD, NIH publication no. 91-3242, pp. 662, 680, 689), Chothia (Chothia et al, 1989, Nature, 342, 877-883) or IMGT (Lefranc, 1999, The Immunologist, 7, 132-136).

Typically, the antibody is a monospecific (i.e., specific for one antigen; such antigen may be common between species or have similar amino acid sequences between species) or bispecific (i.e., specific for two different antigens of a species) antibody comprising at least one HC and LC variable region binding to an antigen target, preferably a membrane bound antigen target which may be internalizing or not internalizing. Preferably, the antibody is internalized by the target cell after binding to the (antigen) target, after which an active effector molecule, which in a conjugate according to the invention is a phosphoantigen, is released intracellularly.

Targeting antibodies, that may be used in conjugates according to the invention for use in cancer therapy, may be a tumor targeting antibody, selectively binding to a tumor-specific or tumor-associated antigen. Tumor-specific antigens only occur on tumor cells, while tumor associated antigens are antigens that are expressed at higher levels (e.g. overexpressed) in cancer cells, when compared to normal (healthy) cells.

The antigen target to which the antibody or antigen binding fragment of a conjugate according to the invention binds may, for example, be selected from the group consisting of: annexin Al, B7H3, B7H4, BCMA, CA6, CA9, CA15-3, CA19-9, CA27-29, CA125, CA242 (cancer antigen 242), CAIX, CCR2, CCR5, CD2, CD19, CD₂O, CD22, CD24, CD30 (tumor necrosis factor 8), CD33, CD37, CD38 (cyclic ADP ribose hydrolase), CD40, CD44, CD47 (integrin associated protein), CD56 (neural cell adhesion molecule), CD70, CD71, CD73, CD74, CD79, CD115 (colony stimulating factor 1 receptor), CD123 (interleukin-3 receptor), CD138 (Syndecan 1), CD203c (ENPP3), CD303, CD333, CDCP1, CEA, CEACAM, Claudin 4, Claudin 7, CLCA-1 (C-type lectin-like molecule-1), CLL 1, c-MET (hepatocyte growth factor receptor), Cripto, DLL3, EGFL, EGFR, EPCAM, EphA2, EPhB3, ETBR (endothelin type B receptor), FAP, FcRL5 (Fc receptor-like protein 5, CD307), FGFR3, FOLR1 (folate receptor alpha), FRbeta, GCC (guanylyl cyclase C), GD2, GITR, GLOBO H, GPA33, GPC3, GPNMB, HER2, p95HER2, HER3, HMW-MAA (high molecular weight melanoma-associated antigen), integrin a (e.g., αvβ3 and αvβ5), IGFIR, TM4SF1 (L6), Lewis A like carbohydrate, Lewis X, Lewis Y (CD174), LGR5, LIV1, mesothelin (MSLN), MN (CA9), MUC1, MUC16, NaPi2b, Nectin-4, Notch3, PD-L1, PSMA, PTK7, SLC44A4, STEAP-1, 5T4 (or TPBG, trophoblast glycoprotein), TF (tissue factor, thromboplastin, CD142), TF-Ag, Tag72, TNFalpha, TNFR, TROP2 (tumor-associated calcium signal transducer 2), uPAR, VEGFR and VLA.

Examples of suitable antibodies known in the art include blinatumomab (CD19), rituximab (CD₂O), or other anti-CD20 antibodies such as ofatumumab, ublituximab or ocrelizumab, epratuzumab (CD22), iratumumab and brentuximab (CD30), gemtuzumab, vadastuximab (CD33), tetulumab (CD37), darartumumab, isatuximab (CD38), bivatuzumab (CD44), alemtuzumab (CD52), lorvotuzumab (CD56), vorsetuzumab (CD70), milatuzumab (CD74), polatuzumab (CD79), rovalpituzumab (DLL3), futuximab (EGFR), oportuzumab (EPCAM), farletuzumab (FOLR1), glembatumumab (GPNMB), trastuzumab, pertuzumab and margetuximab (HER2), etaracizumab (integrin), anetumab (mesothelin), pankomab (MUC1), enfortumab (Nectin-4), H8, A1, and A3 (5T4), and antibodies to TROP2 such as sacituzumab, datopotamab and PF-06664178. An example of a suitable antibody is anti-CD20 antibody rituximab, which was used to exemplify the present invention.

Because pAg activity of the pAg moiety should be displayed in the cell, internalizing antibodies are preferred.

The antibody or antigen-binding fragment thereof, if applicable, may comprise (1) a constant region that is engineered, i.e., one or more mutations may have been introduced to e.g., increase half-life, provide a site of attachment for the linker-drug and/or increase or decrease effector function; or (2) a variable region that is engineered, i.e., one or more mutations may have been introduced to e.g., provide a site of attachment for the linker-drug.

Antibodies or antigen-binding fragments thereof may be produced recombinantly, synthetically, or by other known suitable methods. Mutations that may decrease Fc mediated effector function of antibodies are, for example, mutations such as those described in Leabman et al., 2013, MAbs, 5 (6): 896-903 and Bruhns P, et al., 2015, Immunol Rev., 268 (1): 25-51. doi: 10.1111/imr.12350. PMID: 26497511.

Conjugates according to the present invention may be wild-type or site-specific (meaning a specific conjugation site, such as a cysteine or non-natural amino acid, has been engineered into the antibody protein sequence) or a combination thereof, and can be produced by any method known in the art.

It was found that immunoconjugates according to the invention deliver their pAg payload, to antigen-presenting cells such as cancer cells, very efficiently, resulting in an active phosphoantigen within the antigen-presenting cells. Antigen-presenting cells can be tumor cells, expressing or overexpressing certain tumor antigens on their surface. Such cells may also express or overexpress TCR activating molecules involved in the indirect activation of gammadelta T-cells by pAgs, such as BTN3A1/BTN2A1 receptor complex molecules.

Phosphoantigen Moiety (pAg)

The term “phosphoantigen moiety” or “pAg” as used throughout the present specification refers to pAg moieties with a structural formula represented between the outer brackets in Formula I. In Formula I at least one pAg is conjugated to the linking moiety (L).

A phosphoantigen moiety comprises a non-peptidic antigen with a relatively small mass, that can stimulate gammadelta T-cells (more specifically Vγ9Vδ2 cells) in the presence of antigen-presenting cells.

A “phosphoantigen moiety” as part of a conjugate or linker-drug compound according to the invention, does not necessarily contain a phosphoantigen in its active form. The phosphoantigen moiety in the conjugate or linker-drug compound may comprise an inactive precursor form of an active phosphoantigen and/or may release an active phosphoantigen only after the conjugate binds to its target and has been processed. The phosphoantigen moiety, in its bound state, as part of a conjugate or linker-drug compound, may therefore be structurally different from the active phosphoantigen released therefrom. For example; disconnection from—or cleavage of—a linking moiety may initiate a structural rearrangement and/or a chemical or enzymatic reaction that leads to the formation of a functionally active phosphoantigen. Also the removal- or rearrangement of prodrug moieties, for example in response to changes in the environment or as a result of enzymatic activity at the target site, may release a functionally active phosphoantigen.

In conjugates according to the invention, the specific binding of, e.g., an antibody (targeting moiety), to its specific binding partner (e.g. a tumor specific antigen) will direct a pAg moiety to its target site, not the other way around (the pAg moiety is not the targeting moiety). In a conjugate according to the invention, it is the binding specificity and affinity of the targeting moiety (e.g. the antibody) which ensures that a phosphoantigen moiety is delivered at the site where it has to exert its therapeutic effect.

pAg moieties for use in linker dug compounds and conjugates according to the present invention, comprise an allylic alcohol, or prodrugs thereof (e.g. pAg moieties wherein the allylic alcohol is generated after a prodrug group is removed or after a linker moiety, conjugated through or to the isoprene unit, is cleaved). Such compounds are believed to be examples of pAg moieties comprising direct pAg activity (pAgs that serve as a BTN3A1 ligand).

The activity of a phosphoantigen on Vγ9Vδ2 T cells can be measured in a cellular assay, as is exemplified in the Examples. In the cell based assay used, in a first step, target cells, e.g. tumor cells such as, for example, cells from the CD₂O-positive Burkitt's Lymphoma human tumor cell line Raji, are incubated (overnight) with a phosphoantigen, or a phosphoantigen bearing conjugate according to the invention.

In this first step a phosphoantigen or a conjugate according to the invention will be internalized into the target (tumor) cells. It is assumed that after internalization (and cleavage of the linker in case of a conjugate) the phosphoantigen will bind to the intracellular domain of the BTN3Al receptor, which will lead to activation of the BTN3A1/BTN2A1 dimer.

In a second step the pre-treated, washed, tumor cells from the first step can be cocultured with gammadelta T-cells. When Vγ9Vδ2 T cells become activated, they produce cytokines and release cytotoxic granules (degranulation), leading to immune activation and target cell killing, respectively.

To assess activity of a phosphoantigen on gammadelta T-cells, monensin and/or brefeldin A are added during co-culture of gammadelta T-cells and targets. This will trap produced cytokines (e.g. interferon gamma (IFNγ) and tumor necrosis factor alpha (TNFα)) in activated cells. Staining with fluorescently-labeled antibodies in the presence of saponin, allowing anti-cytokine antibodies to enter the cell, will identify cytokine-producing cells. Fluorescently-labeled antibodies against CD107a can also be added during co-culture and will stain cells that have undergone degranulation. Degranulation correlates with tumor cell killing (Aktas et al., 2009, Cell Immunol., 254 (2), 149-154).

Thus, by combining fluorescently-labeled immune-cell specific markers and CD107a- and cytokine-markers, it is possible to determine the activation status of the gammadelta T-cells and/or other immune cell subsets after co-culture with pretreated target cells.

The ability of gammadelta T-cells to kill pretreated tumor cells can be examined by determining proportions of dead tumor cells after coculture. Tumor cells can be easily identified with a fluorescent tag and their cell dead can already be determined as early as 1 hour after coculture with gammadelta T-cells.

### Phosphoantigen Analogs

(Chemical) analogs are compounds that differ from natural phosphoantigens in their structural characteristics, but resemble natural phosphoantigens in their functional bio-activity. (i.e. they display an (indirect) immune-stimulating activity, in particular, on gammadelta T-cells). Analogs may be designed to improve one or more characteristics of natural occurring pAgs, such as improved characteristics as to stability, potency, bio-availability, or linkage to a linking moiety in the context of their use in immunoconjugates and linker-drug compounds according to the present invention.

### Phosphoantigen Prodrugs

With prodrugs, inactive precursors of phosphoantigen moieties are meant, that are converted into an active phosphoantigen, after the removal or conversion of protective groups (e.g. neutral protecting groups on the negatively charged non-binding oxygen atoms of the phosphonate group(s)). After a conjugate, comprising a phosphoantigen moiety in the form of a prodrug, according to the invention, is administered to the body, the protective groups may be metabolically removed at the target site. A prodrug may also be formed because of binding of the linking moiety to the phosphoantigen moiety. In this case an active phosphoantigen may be formed because the linker in the conjugate, used to bind the phosphoantigen prodrug moiety to the targeting moiety, is cleaved, resulting in the release of an active phosphoantigen, and/or because protective groups are removed from the phosphoantigen moiety. Preferably such conversions, releasing an active phosphoantigen, take place only after a conjugate according to the invention reaches the site where it has to exert its therapeutic effect, for example, after it is internalized by a tumor cell, or at least in the tumor microenvironment, to prevent unwanted and non-specific side effects of a phosphoantigen moiety in healthy and/or non-target tissue.

In a conjugate according to the invention, a phosphoantigen is conjugated to a targeting moiety (e.g. a tumor specific antibody). In a conjugate according to the invention, it is the binding specificity of the targeting moiety which ensures that a phosphoantigen moiety is delivered at the site where it has to exert its therapeutic effect.

Prodrug forms include protecting groups known in the art such as arylesters, aryl amides or pivaloyloxymethyl (POM) prodrug forms. C-HMBP (monophosphonate) phosphoantigen analog/prodrugs are described in WO2019/182904. With the aim to synthesize phosphoantigen prodrugs that are as potent as the natural phosphoantigens such as HMBPP, aryloxy triester phosphoamidite prodrugs of (monophosphonate) phosphoantigens were synthesized, as described in Davey et al., 2018, J. Med. Chem., 61, 2111-2117. In these prodrugs the monophosphonate groups are masked by an aryl motif and an amino acid ester moiety. These compounds (“HMBP ProPagens”) still had rather low serum stability due to the cleavage of the —P—O-bond between the phosphate moiety and the isoprenoid moiety in the molecule. Similar “ProPagens” compounds, wherein the oxygen in the —P—O-bond was replaced by a carbon are described in WO2020/008189. Proposed structure activity relationship (SAR) of phosphoantigen (prodrug) s is described by Wiemer et al., 2020, Chem.Med. Chem., 15, 1030-1039.

A cleavable linking moiety may conveniently be coupled through the alcohol group of an allylalcohol moiety to the phosphoantigen. In this case the allylalcohol may be (re-) formed within the cell when the cleavable linking moiety is cleaved.

Prodrug moieties in a phosphoantigen prodrug as part of a conjugate according to the invention may be the same or different. For example, all prodrug moieties may be POM groups or the phosphoantigen moiety may comprise a combination of, for example, “proTide” groups such as an aryloxy- and an amino acid ester radical, for example such as those described for phosphoantigen prodrugs in WO2020/008189 or WO2019/182904.

Suitable phosphonate prodrug technologies and synthesis of phosphonate prodrugs are known in the art. Such prodrug technologies are further reviewed in, for example, Pradere et al., 2014, Chem. Rev., 114, 9154-9218, and include the use of carbonyloxymethyl prodrug moieties such as pivaloyloxymethyl (POM) and isopropyloxycarbonyloxymethyl (POC) derivatives, S-Acyl-2-thioethyl (SATE) and S-[(2-hydroxyethyl) sulfidyl]-2-thioethyl (DTE) based prodrugs, cyclosaligenyl (cycloSal) phosphate and phosphonate based prodrugs and alkoxyalkyl monoester (hexadecyloxypropyl-(HDP), octadecyloxyethyl-(ODE)) based prodrugs, phosphoramidite and phosphonamidite based prodrugs (including the aryloxy amino acid amidate (ProTide) prodrugs), and phosphordiamidates and phosphonodiamidates.

### Synthesis of Conjugates According to the Invention.

To synthesize a conjugate according to the invention, one or more linker-drug compound(s) according to the invention may be conjugated to a suitable target moiety. When the target moiety is a polypeptide (antibody, or a binding fragment thereof) the linker-drug compound may be conjugated via a reactive native amino acid residue present in the suitable polypeptide, e.g., a lysine or a cysteine, or via an N-terminus or C-terminus. Alternatively, a reactive amino acid residue, natural or non-natural, may be genetically engineered into the suitable polypeptide, or a reactive group may be introduced via post-translational modification.

Conjugates according to the invention may be produced by conjugating a linker-drug compound according to the invention to an antibody or antigen-binding fragment thereof through e.g., the lysine ε-amino groups of the antibody, preferably using an intermediate comprising an amine-reactive group such as an activated ester. Such methods are known for producing conventional Antibody-drug Conjugates (ADCs).

Alternatively, immunoconjugates can be produced by conjugating the linker through the free thiols of the side chains of cysteines generated through reduction of interchain disulfide bonds, using methods and conditions known in the art, see e.g., Doronina et al, 2006, Bioconjugate Chem., 17, 114-124. The manufacturing process involves partial reduction of the solvent-exposed interchain disulfides followed by modification of the resulting thiols with Michael acceptor-containing linkers such as maleimide-containing linkers, alfa-haloacetic amides or esters. The cysteine attachment strategy results in maximally two linker containing linker-drugs per reduced disulfide.

Preferred antibodies used as targeting moieties in conjugates according to the invention are of the human IgG type. Most human IgG molecules have four solvent-exposed disulfide bonds, which equates to a range of integers of from zero to eight linked linking moieties per antibody. The exact number of linked phosphoantigen moieties per target moiety is determined by the number of phosphoantigen moieties per linking moiety, the extent of disulfide reduction and the number of molar equivalents of linker containing linker-drugs in the ensuing conjugation reaction. Full reduction of all four disulfide bonds gives a homogeneous construct with eight linker moieties per antibody, while a partial reduction typically results in a heterogeneous mixture with zero, two, four, six, or eight linking moieties per antibody.

In a preferred embodiment, the present invention relates to a conjugate, wherein the linker-drug compound according to the invention is conjugated to an antibody or antigen-binding fragment thereof through a cysteine residue of the antibody or the antigen-binding fragment.

### Site Specific Conjugation to Antibodies or Antigen Binding Fragments Thereof

Because antibodies contain many lysine residues and cysteine disulfide bonds, conventional conjugation typically produces heterogeneous mixtures that present challenges with respect to analytical characterization and manufacturing. Furthermore, the individual constituents of these mixtures exhibit different physicochemical properties and pharmacology with respect to their pharmacokinetic, efficacy, and safety profiles, hindering a rational approach to optimizing this modality.

To improve conjugate homogeneity, antibodies used in (immuno) conjugates according to the invention may be modified to allow for site-specific conjugation of the linker. Methods for site-specific drug conjugation to antibodies are comprehensively reviewed by C. R. Behrens and B. Liu, 2014, mAbs, 6 (1), 1-8, and can be found in WO2015/177360, WO2005/084390, and WO2006/034488.

Site-specific immunoconjugates are preferably produced by conjugating the linker-drug compound to the antibody or antigen-binding fragment thereof through the side chains of engineered cysteine residues in suitable positions of the mutated antibody or antigen-binding fragment thereof. Engineered cysteines are usually capped by other thiols, such as cysteine or glutathione, to form disulfides. These capped residues need to be uncapped before linker-drug attachment can occur. Linker-drug attachment to the engineered residues is either achieved (1) by reducing both the native interchain and mutant disulfides, then re-oxidizing the native interchain cysteines using a mild oxidant such as CuSO₄ or dehydroascorbic acid, followed by standard conjugation of the uncapped engineered cysteine with a linker-drug, or (2) by using mild reducing agents which reduce mutant disulfides at a higher rate than the interchain disulfide bonds, followed by standard conjugation of the uncapped engineered cysteine with a linker-drug. Suitable methods for site-specifically conjugating linker-drugs can for example be found in WO 2015/177360 which describes the process of reduction and re-oxidation, WO 2017/137628 which describes a method using mild reducing agents and WO 2018/215427 which describes a method for conjugating both the reduced interchain cysteines and the uncapped engineered cysteines.

### Pharmaceutical Compositions

Conjugates according to the invention are intended to be used as a medicament, in particular for the treatment of cancer, autoimmune disease or an infection.

In a further aspect, the invention provides a composition comprising a conjugate according to the invention, preferably wherein the composition is a pharmaceutical composition, more preferably further comprising one or more a pharmaceutically acceptable excipient(s). Such composition is referred to hereinafter as a composition according to the invention. The composition may for example be a liquid formulation, a lyophilized formulation, or in the form of e.g., capsules or tablets.

Typically, pharmaceutical compositions comprising immunoconjugates according to the invention take the form of lyophilized cakes (lyophilized powders), which require (aqueous) dissolution (i.e., reconstitution) before intravenous infusion, or frozen (aqueous) solutions, which require thawing before use. Accordingly, in preferred embodiments, the invention provides a lyophilized composition comprising an immunoconjugate according to the invention, preferably wherein the composition is a pharmaceutical composition, more preferably further comprising one or more pharmaceutically acceptable excipient(s). In further preferred embodiments, the invention provides a frozen composition comprising water and an immunoconjugate according to the invention, preferably wherein the composition is a pharmaceutical composition, more preferably further comprising one or more pharmaceutically acceptable excipient(s). In this context, the frozen solution is preferably at atmospheric pressure, and the frozen solution was preferably obtained by freezing a liquid composition according to the invention at temperatures below 0° C. Suitable pharmaceutically acceptable excipients for inclusion into the pharmaceutical composition (before freeze-drying) in accordance with the present invention include buffer solutions (e.g., citrate, amino acids such as histidine, or succinate containing salts in water), lyoprotectants (e.g., sucrose, trehalose), tonicity modifiers (e.g., chloride salts, such as sodium chloride), surfactants (e.g., polysorbate), and bulking agents (e.g., mannitol, glycine). Excipients used for freeze-dried protein formulations are selected for their ability to prevent protein denaturation during the freeze-drying process as well as during storage.

### Medical Uses

In a further aspect, the invention provides a conjugate according to the invention, or a composition according to the invention, for use as a medicament, preferably for the treatment of cancer, autoimmune or infectious diseases. Conjugates according to the invention can be used to induce a cytotoxic effect of gammadelta T-cells on, for example, tumor- and/or infected cells.

Conjugates and compositions are collectively referred to hereinafter as products for use according to the invention.

In one embodiment, the products for use according to the invention are for use in the treatment of a solid tumor or hematological malignancy. In a second embodiment, the products for use according to the invention are for use in the treatment of an autoimmune disease. In a third embodiment, the products for use according to the invention are for use in the treatment of an infectious disease, such as a bacterial, viral, fungal, parasitic or other infection.

A cancer in the context of the present invention, preferably is a tumor expressing the antigen to which the products for use according to the invention are directed. Such tumor may be a solid tumor or hematological malignancy. Examples of tumors or hematological malignancies that may be treated with products for use according to the invention as defined above may include, but are not limited to, breast cancer; brain cancer (e.g., glioblastoma); head and neck cancer; thyroid cancer; parotic gland cancer, adrenal cancer (e.g., neuroblastoma, paraganglioma, or pheochromocytoma); bone cancer (e.g., osteosarcoma); soft tissue sarcoma (STS); ocular cancer (e.g., uveal melanoma); esophageal cancer; gastric cancer; small intestine cancer; colorectal cancer; urothelial cell cancer (e.g., bladder, penile, ureter, or renal cancer); ovarian cancer; uterine cancer; vaginal, vulvar and cervical cancer; lung cancer (especially non-small cell lung cancer (NSCLC) and small-cell lung cancer (SCLC)); melanoma; mesothelioma (especially malignant pleural and abdominal mesothelioma); liver cancer (e.g., hepatocellular carcinoma); pancreatic cancer; skin cancer (e.g., basalioma, squamous cell carcinoma, or dermatofibrosarcoma protuberans); testicular cancer; prostate cancer; acute myeloid leukemia (AML); chronic myeloid leukemia (CML); chronic lymphatic leukemia (CLL); acute lymphoblastic leukemia (ALL); myelodysplastic syndrome (MDS); blastic plasmacytoid dendritic cell neoplasia (BPDCN); Hodgkin's lymphoma; non-Hodgkin's lymphoma (NHL) (including follicular lymphoma (FL), CNS lymphoma, and diffuse large B-cell lymphoma (DLBCL)); light chain amyloidosis; plasma cell leukemia; and multiple myeloma (MM).

An autoimmune disease in the context of the present invention, preferably is an autoimmune disease associated with the antigen to which the products for use according to the invention are directed. An autoimmune disease represents a condition arising from an abnormal immune response to normal body cells and tissues. There is a wide variety of at least 80 types of autoimmune diseases. Some diseases are organ specific and are restricted to affecting certain tissues, while others resemble systemic inflammatory diseases that impact many tissues throughout the body. The appearance and severity of these signs and symptoms depend on the location and type of inflammatory response that occurs and may fluctuate over time. Examples of autoimmune diseases that may be treated with products for use according to the invention as defined above may include, but are not limited to, rheumatoid arthritis; juvenile dermatomyositis; psoriasis; psoriatic arthritis; lupus; sarcoidosis; Crohn's disease; eczema; nephritis; uveitis; polymyositis; neuritis including Guillain-Barre syndrome; encephalitis; arachnoiditis; systemic sclerosis; autoimmune mediated musculoskeletal and connective tissue diseases; neuromuscular degenerative diseases including Alzheimer's disease, multiple sclerosis (MS), amyotrophic lateral sclerosis (ALS), neuromyelitis optica, and large, middle size, small vessel Kawasaki and Henoch Schonlein vasculitis; cold and warm agglutinin disease; autoimmune hemolytic anemia (AIHA); immune thrombocytopenic purpura ITP), type 1 diabetes mellitus; Hashimoto's thyroiditis; Graves' disease; Graves' ophthalmopathy; adrenalitis; hypophysitis; pemphigus vulgaris; Addison's disease; ankyloses spondylitis; Behcet's syndrome; celiac disease; Goodpasture's syndrome; myasthenia gravis; sarcoidosis; scleroderma; primary sclerosing cholangitis, epidermolysis bullosa acquisita, and bullous pemphigoid.

An infectious disease in the context of the present invention, preferably is an infectious disease associated with the antigen to which the products for use according to the invention are directed. Such infectious disease may be a bacterial, viral, fungal, parasitic or other infection. Examples of infectious diseases that may be treated with products for use according to the invention as defined above may include, but are not limited to, malaria; toxoplasmosis; Pneumocystis jirovecii melioidosis; shigellosis; listeria; diseases caused by Cyclospora or Mycobacterium leprae; tuberculosis; and infectious prophylaxis in immune compromised individuals, such as in HIV-positive individuals, individuals on immunosuppressive treatment, or individuals with inborn errors such as cystic fibrosis or benign proliferative diseases (e.g., mola hydatidosa or endometriosis).

Products for use according to the invention as described herein can be for the use in the manufacture of a medicament as described herein. Products for use according to the invention as described herein are preferably for methods of treatment, wherein the products for use are administered to a subject, preferably to a subject in need thereof, in a therapeutically effective amount. Thus, alternatively, or in combination with any of the other embodiments, in an embodiment, the present invention relates to a use of products for use according to the invention for the manufacture of a medicament for the treatment of cancer, autoimmune or infectious diseases, in particular for the treatment of cancer. For illustrative, non-limitative, cancers or other diseases to be treated according to the invention: see hereinabove.

Alternatively, or in combination with any of the other embodiments, in an embodiment, the present invention relates to a method for treating cancer, autoimmune or infectious diseases, in particular cancer, which method comprises administering to a subject in need of said treatment a therapeutically effective amount of a product for use according to the invention. For illustrative, non-limitative, cancers or other diseases to be treated according to the invention: see hereinabove.

Products for use according to the invention are for administration to a subject. Products for use according to the invention can be used in the methods of treatment described hereinabove by administration of an effective amount of the composition to a subject in need thereof. The term “subject” as used herein refers to all animals classified as mammals and includes, but is not restricted to, primates and humans. The subject is preferably a human. The expression “therapeutically effective amount” means an amount sufficient to effect a desired response, or to ameliorate a symptom or sign. A therapeutically effective amount for a particular subject may vary depending on factors such as the condition being treated, the overall health of the subject, the method, route, and dose of administration and the severity of side effects.

### Combined Use

In further embodiments, the invention provides the product for use according to the invention, wherein the use is combined with one or more other therapeutic agents. Products for use according to the invention may be used concomitantly or sequentially with the one or more other therapeutic agents.

Suitable chemotherapeutic agents include alkylating agents, such as nitrogen mustards, hydroxyurea, nitrosoureas, tetrazines (e.g., temozolomide) and aziridines (e.g., mitomycin); drugs interfering with the DNA damage response, such as PARP inhibitors, ATR and ATM inhibitors, CHK1 and CHK2 inhibitors, DNA-PK inhibitors, and WEE1 inhibitors; anti-metabolites, such as antifolates (e.g., pemetrexed), fluoropyrimidines (e.g, gemcitabine), deoxynucleoside analogues and thiopurines; anti-microtubule agents, such as vinca alkaloids and taxanes; topoisomerase I and II inhibitors; cytotoxic antibiotics, such as anthracyclines and bleomycins; hypomethylating agents such as decitabine and azacitidine; histone deacetylase inhibitors; all-trans retinoic acid; and arsenic trioxide. Suitable radiation therapeutics include radio-isotopes, such as ¹³¹I-metaiodobenzylguanidine (MIBG), ³²P as sodium phosphate, ²²³Ra chloride, ⁸⁹Sr chloride and ¹⁵³Sm diamine tetramethylene phosphonate (EDTMP). Suitable agents to be used as hormonal therapeutics include inhibitors of hormone synthesis, such as aromatase inhibitors and GnRH analogues; hormone receptor antagonists, such as selective estrogen receptor modulators (e.g., tamoxifen and fulvestrant) and antiandrogens, such as bicalutamide, enzalutamide and flutamide; CYP17A1 inhibitors, such as abiraterone; and somatostatin analogs.

Targeted therapeutics are therapeutics that interfere with specific proteins involved in tumorigenesis and proliferation and may be small-molecule drugs; proteins, such as therapeutic antibodies; peptides and peptide derivatives; or protein-small molecule hybrids, such as ADCs. Examples of targeted small molecule drugs include TLR ligands, mTor inhibitors, such as everolimus, temsirolimus and rapamycin; kinase inhibitors, such as imatinib, dasatinib and nilotinib; VEGF inhibitors, such as sorafenib and regorafenib; EGFR/HER2 inhibitors, such as gefitinib, lapatinib, and erlotinib; and CDK4/6 inhibitors, such as palbociclib, ribociclib and abemaciclib. Examples of peptide or peptide derivative targeted therapeutics include proteasome inhibitors, such as bortezomib and carfilzomib.

Suitable anti-inflammatory drugs include D-penicillamine, azathioprine and 6-mercaptopurine, cyclosporine, anti-TNF biologicals (e.g., infliximab, etanercept, adalimumab, golimumab, certolizumab, or certolizumab pegol), lenflunomide, abatacept, tocilizumab, anakinra, ustekinumab, rituximab, daratumumab, ofatumumab, obinutuzumab, secukinumab, apremilast, acetretin, and JAK inhibitors (e.g., tofacitinib, baricitinib, or upadacitinib).

Immunotherapeutic agents include agents that induce, enhance or suppress an immune response, such as cytokines (IL-2 and IFN-α); immuno modulatory imide drugs, e.g., thalidomide, lenalidomide, pomalidomide, or imiquimod; therapeutic cancer vaccines, e.g., talimogene laherparepvec; cell based immunotherapeutic agents, e.g., dendritic cell vaccines, adoptive T-cells, or chimeric antigen receptor-modified T-cells; and therapeutic (bispecific) antibodies, or other ADCs, that can trigger antibody-dependent cell-mediated cytotoxicity (ADCC), antibody-dependent cellular phagocytosis (ADCP) or complement-dependent cytotoxicity (CDC) via their Fc region when binding to membrane bound ligands on a cell.

In the context of the invention, treatment is preferably preventing, reverting, curing, ameliorating, and/or delaying the cancer, autoimmune or infectious disease. This may mean that the severity of at least one symptom of the cancer, autoimmune or infectious disease has been reduced, and/or at least a parameter associated with the cancer, autoimmune or infectious disease has been improved.

In the context of the invention, a subject may survive and/or may be considered as being disease free. Alternatively, the disease or condition may have been stopped or delayed. In the context of the invention, an improvement of quality of life and observed pain relief may mean that a subject may need less pain relief drugs than at the onset of the treatment. “Less” in this context may mean 5% less, 10% less, 20% less, 30% less, 40% less, 50% less, 60% less, 70% less, 80% less, 90% less. A subject may no longer need any pain relief drug. This improvement of quality of life and observed pain relief may be seen, detected or assessed after at least one week, two weeks, three weeks, four weeks, one month, two months, three months, four months, five months, six months or more of treatment in a subject and compared to the quality of life and observed pain relief at the onset of the treatment of said subject.

### General Definitions

Conjugates and linker-drugs according to the invention may contain one or more chiral centers and/or double bonds and therefore, may exist as stereoisomers, such as double-bond isomers (i.e., geometric isomers), regioisomers, enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the person skilled in the art. The compounds may also exist in several tautomeric forms including the enol form, the keto form and mixtures thereof. Accordingly, the chemical structures depicted herein encompass all possible tautomeric forms of the illustrated or identified compounds. It is also understood that some isomeric forms such as diastereomers, enantiomers and geometrical isomers can be separated by physical and/or chemical methods by those skilled in the art. When a structural formula or chemical name is understood by the skilled person to have chiral centers, yet no chirality is indicated, for each chiral center individual reference is made to all three of either the racemic mixture, the pure R enantiomer, and the pure S enantiomer. When the structure of a compound is depicted as a specific enantiomer, it is to be understood that the invention of the present application is not limited to that specific enantiomer. When two moieties are said to together form a bond, this implies the absence of these moieties as atoms, and compliance of valence being fulfilled by a replacing electron bond. All this is known in the art.

The compounds disclosed in this description and in the claims may further exist as exo and endo regioisomers. Unless stated otherwise, the description of any compound in the description and in the claims is meant to include both the individual exo and the individual endo regioisomer of a compound, as well as mixtures thereof. Furthermore, the compounds disclosed in this description and in the claims may exist as cis and trans isomers. Unless stated otherwise, the description of any compound in the description and in the claims is meant to include both the individual cis and the individual trans isomer of a compound, as well as mixtures thereof. As an example, when the structure of a compound is depicted as a cis isomer, it is to be understood that the corresponding trans isomer or mixtures of the cis and trans isomer are not excluded from the invention of the present application.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The word “about” or “approximately” when used in association with a numerical value (e.g., about 10) preferably means that the value may be the given value more or less 1% of the value.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature (RT) to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such charge more often than that it does not bear or carry such charge. As such, an atom that is indicated in this disclosure to be charged could be non-charged under specific conditions, and a neutral moiety could be charged under specific conditions, as is understood by a person skilled in the art.

All patent and literature references cited in the present specification are hereby incorporated by reference in their entirety.

The following Examples are offered for illustrative purposes only, and are not intended to limit the scope of the present invention in any way.

### EXAMPLES

### General Procedures

### Solvents:

All solvents used were reagent grade or HPLC grade from various vendors.

### NMR Spectra:

NMR spectra were recorded on a Bruker AVANCE400 (400 MHz for ¹H; 101 MHz for ¹³C).

### Chemical Shifts:

Chemical shifts are reported in ppm relative to tetramethylsilane as an internal standard, or residual undeuterated solvent.

### UPLC Characterization of Products:

Products were characterized on a Waters UPLC-MS (equipped with an SQD 2 detector) with a Waters ACQUITY UPLC BEH C18 Column (1.7 μm particle size, 2.1×50 mm) at a flow rate of 0.4 mL/min. (MeCN/Water×0.1% Formic acid).

### HPLC Purification:

Purifications by preparative HPLC were performed using a Shimadzu Prominence 20AP system equipped with a Waters SunFire Prep C18 OBD 5 μm column (19×150 mm) at a flow rate of 17 mL/min.

### General Procedure XXD: Pyrophosphate Formation

The triethylamine salt of phosphate XD36 (1.0 equiv.) was dissolved in DMF (0.15 M) under N₂, and CDI (2.1 equiv.) was added at RT. After stirring for 30 min, dry MeOH (1.0 equiv.) was added and the mixture was stirred for 15 min at RT before being concentrated. The residue was coevaporated with DMF to give crude A.

In a separate flask, the mono-triethylamine salt of the phosphate (1.2 equiv.) was coevaporated with DMF and then redissolved in DMF (0.36 M) under N₂. The mixture was then cannulated into the flask containing crude A at RT. An identical volume of DMF was used to rinse the flask and complete the transfer. The mixture was stirred at RT under N₂, and once UPLC-MS analysis showed essentially complete conversion (typically 20-24 h) the reaction was concentrated and purified by preparative HPLC as indicated. Lyophilization of product fractions afforded the product.

### General Procedure XXE: Click-Reaction

Copper(II) sulfate pentahydrate (0.77 equiv.) in nitrogen purged water (0.034 M) was added to a flask containing solid azide (1.0 equiv.) and alkyne (1.4 equiv.) at RT. An equal volume THF was added to give a homogeneous 1:1 water/THF solution. The headspace of the flask was briefly purged with N₂, and a solution of sodium ascorbate (1.5 equiv.) in nitrogen-purged water (0.13 M) was added. The reaction was stirred at RT until UPLC-MS analysis indicated full conversion (typically 1-2 h). Most of the THF was removed by brief rotary evaporation, and the aq. phase was taken up in MeCN/25 mM NH₄HCO₃ in MilliQ (1:9). Insoluble material was filtered off using a syringe filter and the filtrate was purified by preparative HPLC as indicated. Lyophilization of product fractions afforded the product.

General Procedure XXF: Synthesis of Alkyl Phosphates from XD34 and Allylic Alcohols

To a RT solution of alcohol (1.1 equiv), 2,6-lutidine (3.3 equiv.) and 5-(ethylthio)-1H-tetrazole (1.0 equiv.) in MeCN (0.4 M), was added dropwise XD34 (1.0 equiv.) in DCM (0.4 M). The reaction mixture was stirred for 1 to 3 h, and was subsequently quenched with excess MeOH. The reaction mixture was concentrated and partitioned between 1 M HCl (aq) and EtOAc/heptane (1:2). The org. layer was separated and the aq. layer was extracted with EtOAc/heptane (1:2). The combined org. layer was washed with brine, dried over Na₂SO₄ and concentrated. The crude was purified by flash chromatography as indicated.

General Procedure XXG: TBDPS-Deprotection with HF·Pyridine

The TBDPS-ether (1.0 equiv.) was dissolved in THF/pyridine (1:1, 0.2 M) in a PFA tube under a N₂ atmosphere. The solution was cooled to 0° C. and HF·pyridine (13.3 equiv., 70% HF) was slowly added. The reaction mixture was stirred at 0° C. for 60-90 min, and was then carefully added to a sat. aq. NaHCO₃/EtOAc (1:1) mixture under stirring at 0° C. Once effervescence ceased, the layers were separated and the aq. layer was extracted with EtOAc (2×). The combined org. layer was washed with 1 M HCl (aq) and brine, dried over Na₂SO₄ and concentrated. The crude was purified as indicated.

General Procedure XXH: Fluorenylmethyl-Deprotection with Triethylamine

A difluorenylmethyl phosphate (1 equiv.) was dissolved in MeCN/THF (2:1, 0.13 M) and TEA (10 equiv.) was added at RT. The reaction mixture was stirred for 16-20 h. The mixture was allowed to settle and the supernatant was discarded. The oily residue was dissolved in MeCN/MeOH (1:1, 1.0 mL), and Et₂O (10 mL) was slowly added under stirring. The mixture was stirred for 15 min, and after settling, the supernatant was discarded. This process was repeated twice. The residue was coevaporated with MeCN to yield the alkyl phosphate as the triethylammonium salt.

General Procedure XXI: Stille Coupling with XS60

CuI (0.3 equiv.), Pd(PPh₃)₄ (0.15 equiv.) and a tributyl (alkyl) stannane (1.2 equiv.) were purged with N₂ (3×). A solution of XS60 (1.0 equiv.) in toluene (0.11 M) was added and the reaction mixture was stirred at 80° C. for 3-20 h. The mixture was filtered, concentrated and the crude product was purified by flash chromatography as indicated.

General Procedure XXJ: Suzuki Coupling with XS60

XS60 (1.0 equiv.) and Pd(PPh₃)₄ (0.1 equiv.) were purged with N₂ (3×) and dissolved in THF (0.05 M). A suspension of the described zinc-reagent (2.0-4.0 equiv.) was added and the reaction mixture was stirred at RT for 3 h. Sat. aqueous NH₄Cl was added to the reaction mixture and the product was extracted with Et₂O. The combined organic layers were washed with brine, dried over Na₂SO₄ and concentrated. The crude product was filtered from heptane and the filtrate was purified by flash chromatography as indicated.

### General Procedure XXK: THP-Ether Deprotection

A THP-ether (1.0 equiv.) was dissolved in MeOH (0.1 M) and PPTS (0.1 equiv.) was added. The reaction mixture was stirred at 45-55° C. for 3-9 h and was subsequently added to sat. aqueous NaHCO₃. The product was extracted with DCM (3×) and washed with brine, dried over Na₂SO₄ and concentrated. The crude product was purified by flash chromatography as indicated.

General Procedure XXL: Synthesis of Alkyl Phosphates from XD34 and Allylic Alcohols

To a RT solution of alcohol (1.0 equiv), 2,6-lutidine (3.3 equiv.) and 5-(ethylthio)-1H-tetrazole (1.5 equiv.) in MeCN (0.4 M), was added dropwise XD34 (1.5 equiv.) in DCM (0.4 M). The reaction mixture was stirred for 1 to 3 h, and was subsequently quenched with excess MeOH. The reaction mixture was concentrated and partitioned between 1 M HCl (aq) and EtOAc/heptane (1:2). The organic layer was separated and the aqueous layer was extracted with EtOAc/heptane (1:2). The combined organic layer was washed with brine, dried over Na₂SO₄ and concentrated. The crude product was purified by flash chromatography as indicated.

### Example 1: Synthesis of Linker Drug XD78

### 1A: Preparation of Chloride XD68

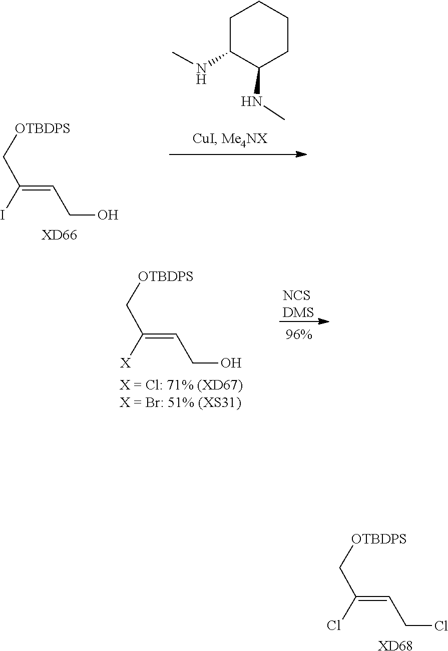

### (Z)-4-((tert-Butyldiphenylsilyl)oxy)-3-chlorobut-2-en-1-ol (XD67)

Prepared according to a procedure by Nitelet, A. et al. Org. Lett. 2016, 18, 1904. A microwave vial was charged with CuI (227 mg, 1.19 mmol), Me₄NCl (872 mg, 7.96 mmol) and iodide XD66 (1.80 g, 3.98 mmol, prepared according to Overman, L. E. Tetrahedron, 2010, 66, 6514). The vial was purged with N₂ and capped. Ethanol (8.0 mL) and (1R,2R)—N1,N2-dimethylcyclohexane-1,2-diamine (340 mg, 2.39 mmol) were added and the vial was heated at 110° C. for 16 h. The reaction was diluted with EtOAc/heptane (1:1, 40 mL) and the suspension was filtered over a silica gel plug. The filtrate was concentrated and the crude was purified by flash chromatography (silica gel, 0-25% ether in heptane), to give alcohol XD67 (1.02 g, 78%) as a colourless oil. ¹H NMR (400 MHz, CDCl₃) ppm=7.73-7.61 (m, 4H), 7.47-7.37 (m, 6H), 6.14 (tt, J=6.3, 1.6 Hz, 1H), 4.35 (tt, J=6.2, 1.3 Hz, 2H), 4.21 (q, J=1.3 Hz, 2H), 1.45-1.39 (m, 1H), 1.08 (s, 9H). MS (ESI⁺) calc, for C₂₀H₂₉ClNO₂Si⁺ [M+NH₄]⁺378.2, found 378.3.

### (Z)-3-Bromo-4-((tert-butyldiphenylsilyl)oxy)but-2-en-1-ol (XS31)

Iodide XD66 (2.00 g, 4.42 mmol, prepared according to Overman, L. E. Tetrahedron, 2010, 66, 6514), was reacted with CuI (253 mg, 1.33 mmol), Me₄NBr (2.72 g, 17.7 mmol) and (1R,2R)—N1,N2-dimethylcyclohexane-1,2-diamine (377 mg, 2.65 mmol) in ethanol (8.9 mL), analogous to the procedure for XD67. The crude was purified by flash chromatography twice (silica gel, 0-20% EtOAc in heptane; silica gel, 0-30% Et₂O in heptane), to give alcohol XS31 (1.79 g, 51%) as a colorless oil. ¹H NMR (400 MHz, DMSO-d₆) ppm=7.67-7.62 (m, 4H), 7.52-7.42 (m, 6H), 6.35-6.30 (m, 1H), 5.04 (t, J=5.6 Hz, 1H), 4.30 (d, J=1.3 Hz, 2H), 4.13-4.08 (m, 2H), 1.03 (s, 9H). MS (ESI⁺) calc, for C₂₀H₂₄BrOSi⁺ [M+H—H₂O]⁺ 387.1, found 387.2.

### (Z)-tert-Butyl ((2,4-dichlorobut-2-en-1-yl)oxy) diphenylsilane (XD68)

N-Chlorosuccinimide (0.621 g, 4.65 mmol) was dissolved in dry DCM (15 mL) and the mixture was cooled to −40° C. Dimethylsulfide (0.424 mL, 5.73 mmol) was added dropwise under stirring, and the mixture was subsequently stirred at 0° C. for 10 min. After cooling to −65° C., alcohol XD67 (1.29 g, 3.58 mmol) in dry DCM (3 mL) was added. The reaction was allowed to warm to 0° C. over 2.5 h, and was then stirred at 0° C. for 90 min. Brine (30 mL) was added at 0° C., and the layers were separated. The aq. layer was extracted with DCM (40 mL) and the combined org. layer was dried over Na₂SO₄. After filtration and concentration, the crude oil was purified by flash chromatography (silica gel, 0-20% DCM in heptane), to give chloride XD68 (1.31 g, 96%) as a colorless oil. ¹H NMR (400 MHz, CDCl₃) ppm=7.70-7.63 (m, 4H), 7.51-7.35 (m, 6H), 6.20 (tt, J=7.6, 1.6 Hz, 1H), 4.28-4.24 (m, 2H), 4.24-4.21 (m, 2H), 1.08 (s, 9H).

### Synthesis of Linker-Drug XD73

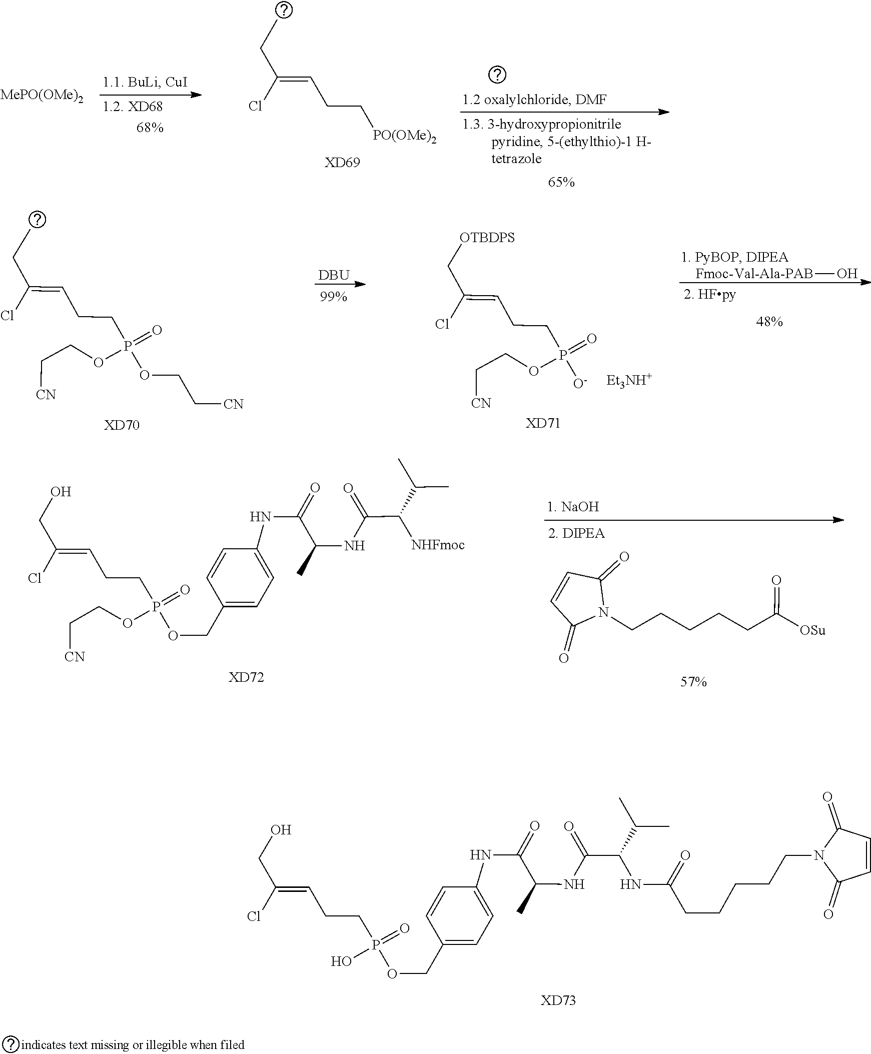

### Dimethyl (Z)-(5-((tert-butyldiphenylsilyl)oxy)-4-chloropent-3-en-1-yl)phosphonate (XD69)

To a solution of dimethyl methylphosphonate (0.817 mL, 7.54 mmol) in THF (28 mL) at −78° C. was added n-BuLi (2.5 M in hexanes, 3.02 mL, 7.54 mmol) under N₂. The reaction was stirred at this temperature for 1 h before being warmed to −50° C. Subsequently, CuI (718 mg, 3.77 mmol) was added and the turbid mixture was stirred for 1 h between −50° C. and −40° C., to give a clear solution. At −40° C., chloride XD68 (1.30 g, 3.43 mmol) in THF (7 mL) was added. The reaction was allowed to warm to RT overnight, and was then quenched with sat. aq. NH₄Cl at 0° C. The aq. layer was extracted with EtOAc (2×) and the combined org. layer was washed with brine, dried over Na₂SO₄, filtered and concentrated in vacuo. Purification by flash chromatography (silica gel, 0-100% EtOAc in heptane) afforded phosphonate XD69 (1.09 g, 68%) as a colorless oil. ¹H NMR (400 MHz, CDCl₃) ppm=7.71-7.59 (m, 4H), 7.51-7.32 (m, 6H), 5.95-5.83 (m, 1H), 4.19 (d, J=1.4 Hz, 2H), 3.76 (s, 3H), 3.74 (s, 3H), 2.57-2.42 (m, 2H), 1.91-1.78 (m, 2H), 1.15-0.99 (m, 9H). MS (ESI⁺) calc. for C₂₃H₃₂ClNaO₄PSi⁺ [M+Na]⁺ 489.1, found 489.4.

### bis(2-Cyanoethyl) (Z)-(5-((tert-butyldiphenylsilyl)oxy)-4-chloropent-3-en-1-yl)phosphonate (XD70)

Step 1: TMSBr (3.03 mL, 23.0 mmol) was added over 10 min to a cooled (0° C.) solution of phosphonate XD69 (1.07 g, 2.30 mmol) in DCM (11 mL). After 30 min, the ice bath was removed and the reaction was stirred at RT for 3 h and 30 min. The mixture was concentrated and the crude was redissolved in DCM (11 mL) under N₂, and cooled to 0° C.

Step 2: DMF (2 drops) was added followed by oxalyl dichloride (0.591 mL, 6.89 mmol). After stirring for 30 min, the ice bath was removed and the mixture was stirred at RT for 16 h. The reaction mixture was concentrated and coevaporated with DCM (2×10 mL). The crude oil was dried at RT under high vacuum for 1 h.

Step 3: The crude oil was dissolved in DCM (16 mL) under N₂ and the mixture was cooled to 0° C. To the mixture was added 5-(ethylthio)-1H-tetrazole (0.149 g, 1.15 mmol), followed by pyridine (0.742 mL, 9.18 mmol) and 3-hydroxypropanenitrile (0.627 mL, 9.18 mmol). After stirring for 2 h at 0° C. and 3 h at RT, the reaction mixture was transferred to separatory funnel and diluted with DCM (20 mL). The organic phase was washed with aq. HCl (1 M, 20 mL), and the aq. phase was back-extracted with DCM (30 mL). The combined org. phase was washed with brine, dried over Na₂SO₄, filtered and concentrated. Purification by flash chromatography (silica gel, 0-100% EtOAc in heptane) afforded phosphonate XD70 (0.811 g, 65%). ¹H NMR (400 MHz, CDCl₃) ppm=7.70-7.61 (m, 4H), 7.48-7.35 (m, 6H), 5.93-5.85 (m, 1H), 4.36-4.22 (m, 4H), 4.21 (d, J=1.3 Hz, 2H), 2.76 (t, J=6.1 Hz, 4H), 2.60-2.48 (m, 2H), 2.00-1.90 (m, 2H), 1.08 (s, 9H). MS (ESI⁺) calc, for C₂₇H₃₅ClN₂O₄PSi⁺ [M+H]⁺ 545.2, found 545.4.

### Triethylammonium 2-cyanoethyl (Z)-(5-((tert-butyldiphenylsilyl)oxy)-4-chloropent-3-en-1-yl)phosphonate (XD71)

DBU (0.247 mL, 1.64 mmol) was added to a solution of phosphonate XD70 (811 mg, 1.49 mmol) in THF (13 mL) at RT. After 30 min, the reaction was concentrated to ~1 mL, diluted with MeOH (6.8 mL), and eluted through a DOWEX 50WX8 plug with methanol (30 mL). Triethylamine (0.228 mL, 1.64 mmol) was added and the mixture was concentrated, and coevaporated with MeCN (2×), to give phosphonate XD71 (840 mg, 99%) as a colorless oil. NMR-analysis indicated a salt ratio of 1:0.8 phosphonate: amine. ¹H NMR (400 MHz, CD₃OD) ppm=7.71-7.65 (m, 4H), 7.48-7.37 (m, 6H), 5.93-5.86 (m, 1H), 4.21 (d, J=1.1 Hz, 2H), 4.05 (dt, J=7.1, 6.2 Hz, 2H), 3.20 (q, J=7.3 Hz, 5H), 2.77 (t, J=6.1 Hz, 2H), 2.54-2.41 (m, 2H), 1.73-1.60 (m, 2H), 1.31 (t, J=7.3 Hz, 7H), 1.06 (s, 9H). MS (ESI⁻) calc. for C₂₄H₃₀ClNO₄PSi⁻ [M−H]⁻ 490.1, found 490.4.

### Phosphonate XD72

Step 1: Triethylamine salt XD71 (830 mg, 1.45 mmol) and Fmoc-Val-Ala-PAB-OH (898 mg, 1.74) were coevaporated with dry DMF (3×8 mL). DMF (7 mL) was added) at RT under N₂, followed by PyBOP (906 mg, 1.74 mmol) and DIPEA (0.507 mL, 2.90 mmol. After 2 h, the reaction was then slowly added, in a dropwise fashion, to ice-cold water (70 mL) under very gentle stirring (to suppress gel formation). The white suspension was gently stirred for 5 min, and was then filtered. The solid was collected and residual water was removed by coevaporation with MeCN (2×). Purification of the crude solid by flash chromatography (silica gel, 0-6% MeOH in DCM) afforded the intermediate phosphonate (1.10 g, 77%).

Step 2: To a nitrogen flushed PFA vial containing the intermediate phosphonate (1.10 g, 1.11 mmol) prepared above, was added THF (6.2 mL) and pyridine (3.1 mL). HF-pyridine (70% HF, 1.5 mL) was introduced by syringe under N₂ at 0° C., and the mixture was stirred for 90 min. at 0° C. The reaction mixture was carefully transferred via cannula to cold (0° C.) sat. aq. NaHCO₃ (150 mL) under gentle stirring. After stirring for 15 min, the suspension was filtered and the white solid was washed with water (2×10 mL), collected from the filter and coevaporated with MeCN (2×15 mL). The solid was dried under vacuum ON and was then purified by flash chromatography (silica gel, 0-8% MeOH in DCM) to give phosphonate XD72 (516 mg, 62%) as a white foam. ¹H NMR (400 MHz, CD₃OD) ppm=7.79 (dd, J=7.5, 0.8 Hz, 2H), 7.70-7.59 (m, 4H), 7.43-7.35 (m, 4H), 7.33-7.28 (m, 2H), 5.88 (t, J=7.1 Hz, 1H), 5.10 (d, J=12.5 Hz, 1H), 5.06 (d, J=12.5 Hz, 1H), 4.49 (q, J=7.1 Hz, 1H), 4.44-4.34 (m, 2H), 4.25-4.07 (m, 3H), 4.06 (s, 2H), 3.95 (d, J=7.0 Hz, 1H), 2.79 (t, J=5.9 Hz, 2H), 2.47 (dq, J=15.1, 7.4 Hz, 2H), 2.08 (dq, J=13.6, 6.8 Hz, 1H), 2.01-1.89 (m, 2H), 1.44 (d, J=7.1 Hz, 3H), 0.98 (d, J=6.9 Hz, 3H), 0.96 (d, J=6.8 Hz, 3H). MS (ESI⁺) calc. for C₃₈H₄₅ClN₄O₈P⁺ [M+H]⁺ 751.3, found 751.8.

### 1B: Linker-Drug XD73

Step 1: To a suspension of phosphonate XD72 (0.117 g, 0.156 mmol) in MeOH (2.3 mL) and water (0.26 mL) at 0° C., was added aq. NaOH (2.0 M, 0.389 mL, 0.779 mmol). The cooling bath was removed and the mixture was stirred for 100 min. The reaction was cooled to 0° C. and AcOH (2 M in MeOH, 0.623 mL, 1.25 mmol) was added. Methanol was removed by rotary evaporation and the suspension was diluted with water (2 mL) and filtered. The solid was washed with water and the aq. filtrate was lyophilized to give the crude amine as a glassy solid. The material was carried forward without further purification.

Step 2: The crude amine was dissolved/suspended in DMF (1 mL). DIPEA (0.108 mL, 0.622 mmol) was added at RT followed by 2,5-dioxopyrrolidin-1-yl 6-(2,5-dioxo-2,5-dihydro-1H-pyrrol-1-yl) hexanoate (0.096 g, 0.311 mmol). The reaction was stirred at RT for 2 h at which point acetic acid (0.053 mL, 0.933 mmol) in DMF (1 mL) was added, and the mixture was concentrated. The residue was dissolved in 1:9 MeCN/aq. NH₄HCO₃ (25 mM), and the solution was washed with EtOAc (4×5 mL) and ether (1×5 mL). The aq. phase was then directly purified by preparative RP-HPLC (25 mM NH₄HCO₃ in MilliQ/MeCN, gradient 90:10 to 40:60). Lyophilization of product fractions afforded linker-drug XD73 (60.5 mg, 57%) as a white solid. ¹H NMR (400 MHz, DMSO-d₆) ppm=9.96 (s, 1H), 8.18 (d, J=6.8 Hz, 1H), 7.86 (d, J=8.5 Hz, 1H), 7.55 (d, J=8.6 Hz, 2H), 7.28 (d, J=8.6 Hz, 2H), 6.95 (s, 2H), 5.87 (t, J=7.0 Hz, 1H), 4.76 (d, J=7.1 Hz, 2H), 4.35 (q, J=7.1 Hz, 1H), 4.12 (d, J=6.9 Hz, 1H), 3.92 (br s, 2H), 3.35 (t, J=7.1 Hz, 2H), 2.35-2.22 (m, 2H), 2.21-2.05 (m, 2H), 2.02-1.87 (m, 1H), 1.62-1.50 (m, 2H), 1.50-1.38 (m, 4H), 1.29 (d, J=7.1 Hz, 3H), 1.15 (quint, J=7.6 Hz, 2H), 0.84 (d, J=6.8 Hz, 3H), 0.80 (d, J=6.8 Hz, 3H). MS (ESI⁻) calc. for C₃₀H₄₁ClN₄O₉P⁻ [M−H]⁻ 667.2, found 667.7.

### Example 2: Synthesis of Linker Drugs XS54-XS58

### 2A. Synthesis of Alcohols XS3 and XS38

### Synthesis of Alcohol XS33

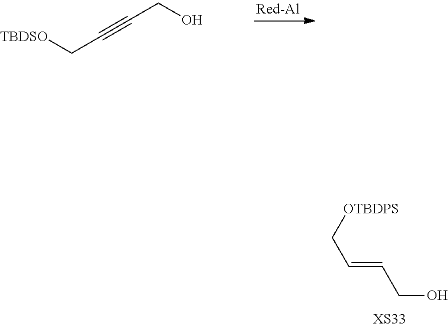

### (E)-4-((tert-Butyldiphenylsilyl)oxy)but-2-en-1-ol (XS33)

To a 0° C. suspension of Red-Al (3.17 mL, 70% in toluene, 11.2 mmol) in Et₂O (26 mL) was dropwise added a solution of 4-((tert-butyldiphenylsilyl)oxy)but-2-yn-1-ol (1.75 g, 5.39 mmol, prepared as described in Trost and Livingston, J. Am. Chem. Soc. 2008, 130, 11970-11978) in Et₂O (5.2 mL). The reaction mixture was stirred at 0° C. for 90 min and EtOAc (0.528 mL, 5.39 mmol) was added. Stirring was continued at 0° C. for 30 min, after which the reaction was quenched by addition of 1 M aq. sodium potassium tartrate (5.4 mL). The mixture was stirred at 0° C. for 30 min, diluted with water (5.4 mL) and added to heptane (30 mL). The organic layer was washed with brine (20 mL), dried over Na₂SO₄ and concentrated. Purification by flash chromatography (silica gel, 0-25% EtOAc in heptane) afforded allyl alcohol XS33 (1.39 g, 79%) as a colorless oil. ¹H NMR (400 MHz, DMSO-d₆) ppm=7.65-7.61 (m, 4H), 7.50-7.40 (m, 6H), 5.83 (dtt, J=15.4, 4.9, 1.5 Hz, 1H), 5.72 (dtt, J=15.4, 4.4, 1.5 Hz, 1H), 4.74 (t, J=5.4 Hz, 1H), 4.22-4.17 (m, 2H), 3.99-3.92 (m, 2H), 1.00 (s, 9H). MS (ESI⁺) calc, for C₂₀H₂₅OSi⁺ [M+H−H₂O]⁺ 309.2, found 309.3.

Synthesis of alcohol XS38

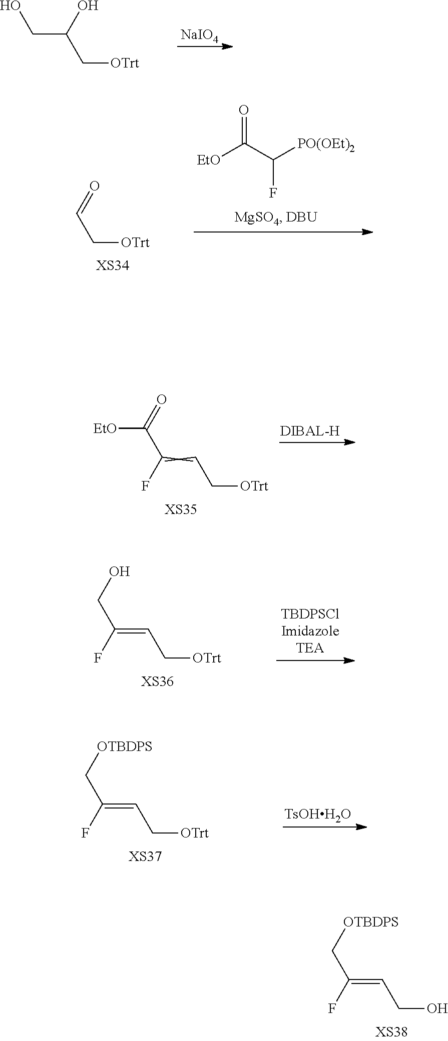

### 2-(Trityloxy) acetaldehyde (XS34)

Silica gel-supported NaIO₄ (22.3 g, 15.2 mmol, prepared as described in Zong and Shing, J. Org. Chem. 1997, 62, 2622-2624) was purged with N₂ (3×) and suspended in DCM (50 mL), and 3-(trityloxy) propane-1,2-diol (3.74 g, 11.2 mmol, prepared as described in CN108478807) dissolved in DCM (25 mL) was added. The reaction mixture was stirred at RT for 2 h. The reaction mixture was filtered over a silica gel plug and was washed with DCM (100 mL). The filtrate was concentrated and the crude was purified by flash chromatography (silica gel, 0-25% EtOAc in heptane), to give aldehyde XS34 (2.88 g, 85%) as a colorless oil. ¹H NMR (400 MHz, DMSO-d₆) ppm=9.36 (s, 1H), 7.44-7.31 (m, 15H), 3.85 (s, 2H).

### Ethyl 2-fluoro-4-(trityloxy)but-2-enoate (XS35)

A flask containing aldehyde XS34 (2.77 g, 9.16 mmol) and MgSO₄ (1.33 g, 11.1 mmol) was purged with N₂ (3×), and MeCN (46 mL) was added. The mixture was cooled to 0° C. and ethyl 2-(diethoxyphosphoryl)-2-fluoroacetate (2.29 mL, 11.3 mmol) was added, followed by DBU (1.38 mL, 9.16 mmol). The reaction mixture was allowed to reach RT and stirred for 2 h. The reaction mixture was concentrated and the remainder was taken up in EtOAc (100 mL), washed with 1 M HCl (2×50 mL), 2 M NaOH (2×50 mL) and brine (50 mL), dried over Na₂SO₄ and concentrated. Purification by flash chromatography (silica gel, 0-15% EtOAc in heptane) afforded vinyl fluoride XS35 (2.92 g, 82%) as a mixture of Z:E isomers (2:1 ratio). MS (ESI⁺) calc, for C₂₅H₂₃FNaO₃⁺ [M+Na]⁺ 413.2, found 413.4.

### (Z)-2-Fluoro-4-(trityloxy)but-2-en-1-ol (XS36)

Ester XS35 (2.85 g, 7.30 mmol, 2:1 Z:E ratio) was purged with N₂ (3×) and dissolved in THF (24 mL). The solution was cooled to −78° C. and DIBAL-H (21.9 mL, 1 M in toluene, 21.9 mmol) was dropwise added. After 15 min, the reaction mixture was allowed to reach RT and was stirred for 2 h. The reaction mixture was cooled to 0° C. and quenched by addition of 15% aq. citric acid (25 mL). The mixture was diluted with water (75 mL) and the product was extracted with EtOAc (3×100 mL). The combined org. layers were washed with brine (100 mL), dried over Na₂SO₄ and concentrated. Purification by flash chromatography (silica gel, 0-35% EtOAc in heptane) afforded alcohol XS36 (1.54 g, 60%) as a colorless oil. ¹H NMR (400 MHz, DMSO-d₆) ppm=7.41-7.32 (m, 12H), 7.30-7.24 (m, 3H), 5.32 (t, J=5.9 Hz, 1H), 5.19 (dt, J=37.8, 7.0 Hz, 1H), 3.94 (dd, J=12.9, 5.6 Hz, 2H), 3.58 (dd, J=6.8, 1.0 Hz, 2H). MS (ESI⁺) calc, for C₂₃H₂₁FNaO₂⁺ [M+Na]⁺ 371.1, found 371.3. (E)-2-fluoro-4-(trityloxy)but-2-en-1-ol (0.777 g, 31%) was also isolated. ¹H NMR (400 MHz, DMSO-d₆) ppm=7.41-7.32 (m, 12H), 7.31-7.24 (m, 3H), 5.31 (dt, J=20.1, 7.6 Hz, 1H), 5.17 (t, J=5.6 Hz, 1H), 3.86 (dd, J=22.1, 5.6 Hz, 2H), 3.56 (dd, J=7.8, 1.0 Hz, 2H). MS (ESI⁺) calc. for C₂₃H₂₁FNaO₂⁺ [M+Na]⁺ 371.1, found 371.3.

### (Z)-tert-Butyl ((2-fluoro-4-(trityloxy)but-2-en-1-yl)oxy) diphenylsilane (XS37)

Alcohol XS36 (1.53 g, 4.38 mmol) was dissolved in DCM (29 mL) under N₂. The solution was cooled to 0° C., and TEA (1.22 mL, 8.76 mmol) and imidazole (0.328 g, 4.82 mmol) were added, followed by the dropwise addition of TBDPS-C₁ (1.69 mL, 6.57 mmol). After 15 min, the reaction mixture was allowed to reach R^{T} and was stirred for 2 h. The reaction mixture was added to water (30 mL), the org. layer was separated and the water was extracted with DCM (30 mL). The combined org. layers were washed with (20 mL), dried over Na₂SO₄ and concentrated. Purification by flash chromatography (silica gel, 0-5% EtOAc in heptane) afforded silyl ether XS37 (2.47 g, 96%) as a colorless oil. ¹H NMR (400 MHz, DMSO-d₆) ppm=7.67-7.59 (m, 4H), 7.53-7.40 (m, 6H), 7.39-7.31 (m, 12H), 7.30-7.23 (m, 3H), 5.17 (dt, J=37.3, 6.9 Hz, 1H), 4.21 (d, J=13.4 Hz, 2H), 3.59 (d, J=6.9 Hz, 2H), 1.01 (s, 9H).

### (Z)-4-((tert-Butyldiphenylsilyl)oxy)-3-fluorobut-2-en-1-ol (XS38)

Trityl ether XS37 (1.00 g, 1.70 mmol) was dissolved in DCM (8.5 mL) and MeOH (8.5 mL) under N₂. The solution was cooled to 0° C. and TsOH·H₂O (0.486 g, 2.56 mmol) was added. The reaction mixture was stirred at 0° C. for 1 h. The reaction mixture was added to sat. aq. NaHCO₃ (50 mL) and the product was extracted with DCM (3×50 mL). The combined org. layers were washed with brine (50 mL), dried over Na₂SO₄ and concentrated. Purification by flash chromatography (silica gel, 0-25% EtOAc in heptane) afforded alcohol XS38 (0.462 g, 79%) as a colorless oil. ¹H NMR (400 MHz, DMSO-d₆) ppm=7.66-7.60 (m, 4H), 7.52-7.41 (m, 6H), 5.12 (dt, J=38.0, 6.9 Hz, 1H), 4.78 (t, J=5.6 Hz, 1H), 4.20 (d, J=13.0 Hz, 2H), 4.05-3.99 (m, 2H), 1.01 (s, 9H). MS (ESI⁺) calc, for C₂₀H₂₉FNO₂Si⁺ [M+H]⁺362.2, found 362.3.

### 2B. Preparation of bis((9H-fluoren-9-yl)methyl) phosphorochloridate (XD34)

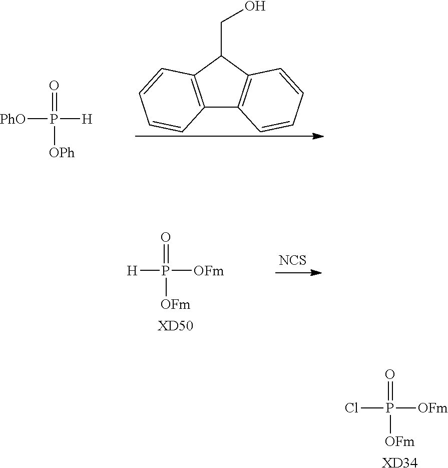

### bis((9H-Fluoren-9-yl)methyl) phosphonate (XD50)

(9H-Fluoren-9-yl) methanol (4.55 g, 23.2 mmol) was added to a solution of diphenyl phosphite (2.13 mL, 10.6 mmol) in dry pyridine (20 mL) at RT under N₂, and the mixture was stirred for 2 h. The reaction was concentrated and taken up in EtOAc (250 mL). The organic phase was washed with aq. HCl (2×, 1 M) and brine, dried over Na₂SO₄, filtered and concentrated on silica gel. Purification by flash chromatography (silica gel, 0-85% EtOAc/DCM (1:4) in heptane) afforded H-phosphonate XD50 (3.46 g, 75%) as a colorless wax. ¹H NMR (400 MHz, CDCl₃) ppm=7.76-7.66 (m, 4H), 7.58-7.45 (m, 4H), 7.42-7.31 (m, 4H), 7.31-7.22 (m, 4H), 7.19-7.12 (m, 1H), 6.68 (d, J=705.8 Hz, 1H), 4.34-4.21 (m, 4H), 4.15-4.08 (m, 2H). MS (ESI⁺) calcd, for C₂₈H₂₄O₃P⁺ [M+H]⁺ 439.2 found 439.3.

### bis((9H-Fluoren-9-yl)methyl) phosphorochloridate (XD34)

H-phosphonate XD50 (8.57 g, 19.6 mmol) was dissolved in toluene (98 mL) and the overhead space was purged with N₂. NCS (3.13 g, 23.5 mmol) was added at RT and the reaction mixture was then stirred at 40° C. for 2 h. After cooling to RT, the reaction mixture was filtered and concentrated. The residue was coevaporated with MeCN (10 mL) to afford a white solid. The solid was dissolved in MeCN (25 mL) using gentle heating with a heat gun to dissolve all the solid. The solution was gradually cooled down to −30° C. at which point a white solid started to precipitate. The flask was stored at −30° C. overnight and was then allowed to warm to RT before filtration. Ice-cold MeCN (10 mL) was used to wash the solid to give chloride XD34 (8.03 g, 87% yield) as a white solid. ¹H NMR (400 MHz, CDCl₃) ppm=7.76-7.71 (m, 4H), 7.56-7.48 (m, 4H), 7.43-7.36 (m, 4H), 7.33-7.25 (m, 4H), 4.46 (dt, J=9.7, 7.1 Hz, 2H), 4.36-4.28 (m, 2H), 4.25-4.19 (m, 2H). MS (ESI⁺) calcd. for C₂₈H₂₄ClO₄P⁺ [M+NH₄]⁺ 490.1 found 490.3.

### 2C. Synthesis of phosphates XS44-XS48

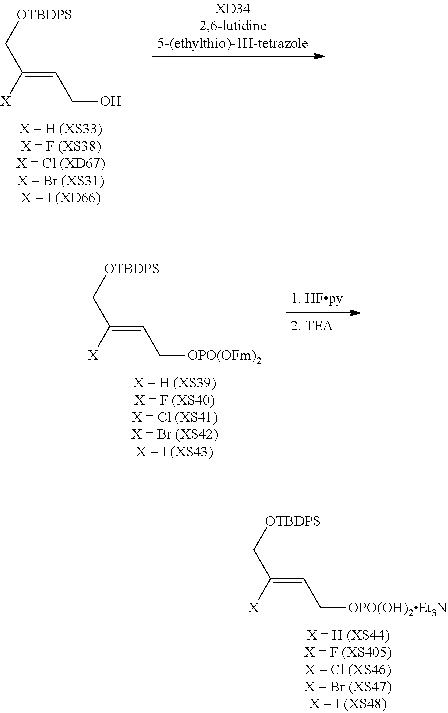

### (E)-bis((9H-Fluoren-9-yl)methyl) (4-((tert-butyldiphenylsilyl)oxy)but-2-en-1-yl) phosphate (XS39)

Allylic alcohol XS33 was reacted with XD34 (0.500 g, 1.06 mmol) according to general procedure XXF. The crude was purified by flash chromatography (silica gel, 0-40% EtOAc in heptane) to yield alkyl phosphate XS39 (0.458 g, 57%) as a white sticky solid. ¹H NMR (400 MHz, DMSO-d₆) ppm=7.84 (dd, J=7.5, 4.1 Hz, 4H), 7.61-7.48 (m, 8H), 7.47-7.33 (m, 10H), 7.26 (tdd, J=7.4, 5.2, 1.0 Hz, 4H), 5.76-5.62 (m, 2H), 4.29-4.13 (m, 8H), 4.13-4.09 (m, 2H), 0.95 (s, 9H). MS (ESI⁺) calc, for C₄₈H₄₈O₅PSi⁺ [M+H]⁺ 763.3, found 763.6.

### (Z)-bis((9H-Fluoren-9-yl)methyl) (4-((tert-butyldiphenylsilyl)oxy)-3-fluorobut-2-en-1-yl) phosphate (XS40)

Allylic alcohol XS38 was reacted with XD34 (0.570 g, 1.21 mmol) according to general procedure XXF. The crude was purified by flash chromatography (silica gel, 0-40% EtOAc in heptane) to yield alkyl phosphate XS40 (0.555 g, 59%) as a white sticky solid. ¹H NMR (400 MHz, DMSO-d₆) ppm=7.83 (dd, J=7.5, 3.8 Hz, 4H), 7.63-7.31 (m, 18H), 7.30-7.21 (m, 4H), 5.02 (dt, J=35.8, 7.4 Hz, 1H), 4.28-4.19 (m, 6H), 4.18-4.09 (m, 4H), 0.95 (s, 9H). MS (ESI⁺) calc, for C₄₈H₄₇FO₅PSi⁺ [M+H]⁺ 781.3, found 781.7.

### (Z)-bis((9H-Fluoren-9-yl)methyl) (4-((tert-butyldiphenylsilyl)oxy)-3-chlorobut-2-en-1-yl) phosphate (XS41)

Allylic alcohol XD67 was reacted with XD34 (0.378 g, 0.799 mmol) according to general procedure XXF. The crude was purified by flash chromatography (silica gel, 0-40% EtOAc in heptane) to yield alkyl phosphate XS41 (0.426 g, 67%) as a white sticky solid. ¹H NMR (400 MHz, DMSO-d₆) ppm=7.84 (dd, J=7.6, 3.8 Hz, 4H), 7.60-7.34 (m, 18H), 7.30-7.22 (m, 4H), 5.91 (t, J=6.3 Hz, 1H), 4.35-4.21 (m, 6H), 4.19-4.13 (m, 4H), 0.96 (s, 9H). MS (ESI⁺) calc, for C₄₈H₄₇C₁₀₅PSi⁺ [M+H]⁺ 797.3, found 797.9.

### (Z)-bis((9H-Fluoren-9-yl)methyl) (3-bromo-4-((tert-butyldiphenylsilyl)oxy)but-2-en-1-yl) phosphate (XS42)

Allylic alcohol XS31 was reacted with XD34 (0.500 g, 1.06 mmol) according to general procedure XXF. The crude was purified by flash chromatography (silica gel, 0-35% EtOAc in heptane) to yield alkyl phosphate XS42 (0.890 g, 51%) as a colorless oil. ¹H NMR (400 MHz, DMSO-d₆) ppm=7.84 (dd, J=7.5, 3.8 Hz, 4H), 7.62-7.57 (m, 4H), 7.53 (dd, J=16.3, 7.5 Hz, 4H), 7.48-7.31 (m, 10H), 7.30-7.24 (m, 4H), 6.15 (t, J=6.0 Hz, 1H), 4.33-4.19 (m, 8H), 4.19-4.13 (m, 2H), 0.96 (s, 9H).

### (Z)-bis((9H-Fluoren-9-yl)methyl) (4-((tert-butyldiphenylsilyl)oxy)-3-iodobut-2-en-1-yl) phosphate (XS43)

Allylic alcohol XD66 was reacted with XD34 (0.350 g, 0.740 mmol) according to general procedure XXF. The crude was purified by flash chromatography (silica gel, 0-35% EtOAc in heptane) to yield alkyl phosphate XS43 (0.430 g, 65%) as a white sticky solid. ¹H NMR (400 MHz, DMSO-d₆) ppm=7.84 (dd, J=7.5, 3.8 Hz, 4H), 7.61-7.50 (m, 8H), 7.47-7.33 (m, 10H), 7.31-7.22 (m, 4H), 6.12 (t, J=5.8 Hz, 1H), 4.30-4.12 (m, 10H), 0.97 (s, 9H). MS (ESI⁺) calc, for C₄₈H₄₇IO₅PSi⁺ [M+H]⁺ 889.2, found 889.7.

Triethylammonium (E)-4-hydroxybut-2-en-1-yl phosphate (XS44) Step 1: TBDPS-ether XS39 (0.455 g, 0.596 mmol) was reacted according to general procedure XXG. The crude was purified by flash chromatography (silica gel, 0-75% EtOAc in heptane) to yield the intermediate allylic alcohol (47 mg, 15%) as a white solid. ¹H NMR (400 MHz, DMSO-d₆) ppm=7.87 (t, J=6.7 Hz, 4H), 7.53 (dd, J=17.1, 7.5 Hz, 4H), 7.39 (dt, J=11.8, 7.3 Hz, 4H), 7.34-7.25 (m, 4H), 5.74-5.65 (m, 1H), 5.59-5.50 (m, 1H), 4.79 (t, J=5.4 Hz, 1H), 4.27-4.11 (m, 8H), 3.92-3.85 (m, 2H). MS (ESI⁺) calc, for C₃₂H₃₀O₅P⁺ [M+H]⁺ 525.2, found 525.4.

Step 2: The intermediate (47 mg, 0.090 mmol) was reacted according to general procedure XXH, to yield alkyl phosphate XS44 (14 mg, 61%) as the triethylamine salt. MS (ESI⁻) calc, for C₄H₈O₅P⁻ [M−H]⁻ 167.0, found 166.9.

Triethylammonium (Z)-3-fluoro-4-hydroxybut-2-en-1-yl phosphate (XS45) Step 1: TBDPS-ether XS40 (0.552 g, 0.707 mmol) was reacted according to general procedure XXG. The crude was purified by flash chromatography (silica gel, 0-60% EtOAc in heptane) to yield the intermediate allylic alcohol (0.355 g, 93%) as a colorless oil. ¹H NMR (400 MHz, DMSO-d₆) ppm=7.86 (t, J=6.8 Hz, 4H), 7.53 (dd, J=16.6, 7.5 Hz, 4H), 7.39 (dt, J=12.1, 7.4 Hz, 4H), 7.34-7.24 (m, 4H), 5.37 (t, J=5.9 Hz, 1H), 4.99 (dt, J=36.1, 7.4 Hz, 1H), 4.29-4.19 (m, 6H), 4.19-4.13 (m, 2H), 3.91 (dd, J=12.1, 5.9 Hz, 2H). MS (ESI⁺) calc, for C₃₂H₂₉FO₅P⁺ [M+H]⁺ 543.2, found 543.2.

Step 2: The intermediate (0.350 g, 0.645 mmol) was reacted according to general procedure XXH, to yield alkyl phosphate XS45 (0.115 g, 69%) as the triethylamine salt in a 1:0.7 ratio of phosphate:Et₃N. ¹H NMR (400 MHz, DMSO-d₆) ppm=5.11 (dt, J=37.9, 7.0 Hz, 1H), 4.30-4.24 (m, 2H), 3.93 (d, J=13.1 Hz, 2H), 2.94 (q, J=7.3 Hz, 4H), 1.15 (t, J=7.3 Hz, 6H). MS (ESI⁻) calc, for C₄H₇FO₅P⁻ [M−H]⁻ 185.0, found 185.0.

### Triethylammonium (Z)-3-chloro-4-hydroxybut-2-en-1-yl hydrogen phosphate (XS46)

Step 1: TBDPS-ether XS41 (0.422 g, 0.529 mmol) was reacted according to general procedure XXG. The crude was suspended in DCM (10 mL) and to this were added Et₂O (40 mL) and heptane (50 mL). The suspension was filtered after gentle heating and the residue was washed with Et₂O (10 mL), to yield the intermediate allylic alcohol (0.202 g, 68%) as a white solid. ¹H NMR (400 MHz, DMSO-d₆) ppm=7.86 (t, J=6.6 Hz, 4H), 7.54 (dd, J=17.3, 7.5 Hz, 4H), 7.39 (dt, J=11.6, 7.3 Hz, 4H), 7.33-7.25 (m, 4H), 5.92-5.86 (m, 1H), 5.58 (t, J=6.1 Hz, 1H), 4.33-4.21 (m, 6H), 4.19-4.13 (m, 2H), 3.96 (dd, J=6.1, 1.1 Hz, 2H). MS (ESI⁺) calc, for C₃₂H₂₉ClO₅P⁺ [M+H]⁺ 559.1, found 559.4.

Step 2: The intermediate (0.198 g, 0.354 mmol) was reacted according to general procedure XXH, to yield alkyl phosphate XS46 (53 mg, 49%) as the triethylamine salt in a 1:0.9 ratio of phosphate:Et₃N. ¹H NMR (400 MHz, DMSO-d₆) ppm=6.03 (tt, J=5.8, 1.3 Hz, 1H), 4.38-4.32 (m, 2H), 3.98 (d, J=1.3 Hz, 2H), 2.94 (q, J=7.0 Hz, 5H), 1.15 (t, J=7.3 Hz, 8H). MS (ESI⁻) calc, for C₄H₇ClO₅P⁻ [M−H]⁻ 201.0, found 200.7.

### Triethylammonium (Z)-3-bromo-4-hydroxybut-2-en-1-yl phosphate (XS47)

Step 1: TBDPS-ether XS42 (0.445 g, 0.529 mmol) was reacted according to general procedure XXG. The crude was purified by flash chromatography (silica gel, 0-60% EtOAc in heptane) to yield the intermediate allylic alcohol (0.294 g, 92%) as a white solid. ¹H NMR (400 MHz, DMSO-d₆) ppm=7.86 (t, J=6.6 Hz, 4H), 7.54 (dd, J=16.9, 7.5 Hz, 4H), 7.39 (dt, J=11.8, 7.4 Hz, 4H), 7.34-7.24 (m, 4H), 6.16-6.09 (m, 1H), 5.63 (t, J=6.2 Hz, 1H), 4.32-4.22 (m, 6H), 4.20-4.14 (m, 2H), 4.02 (dd, J=6.1, 1.4 Hz, 2H). MS (ESI⁺) calc. for C₃₂H₂₉BrO₅P⁺ [M+H]⁺ 603.1, found 603.4.

Step 2: The intermediate (0.290 g, 0.481 mmol) was reacted according to general procedure XXH, to yield alkyl phosphate XS47 (0.130 g, 78%) as the triethylamine salt in a 1:0.95 ratio of phosphate:Et₃N. ¹H NMR (400 MHz, D₂O) ppm=6.18 (t, J=5.9 Hz, 1H), 4.47-4.39 (m, 2H), 4.15 (d, J=0.9 Hz, 2H), 3.08 (q, J=7.4 Hz, 6H), 1.15 (t, J=7.4 Hz, 9H). MS (ESI⁻) calc, for C₄H₇BrO₅P⁻ [M−H] 244.9, found 245.1.

### Triethylammonium (Z)-4-hydroxy-3-iodobut-2-en-1-yl hydrogen phosphate (XS48)

Step 1: TBDPS-ether XS43 (0.423 g, 0.476 mmol) was reacted according to general procedure XXG. The crude was purified by flash chromatography (silica gel, 0-60% EtOAc in heptane) to yield the intermediate allylic alcohol (0.262 g, 85%) as a white solid. ¹H NMR (400 MHz, DMSO-d₆) ppm=7.87 (t, J=6.8 Hz, 4H), 7.54 (dd, J=15.1, 7.4 Hz, 4H), 7.39 (dt, J=12.1, 7.3 Hz, 4H), 7.33-7.25 (m, 4H), 6.13-6.08 (m, 1H), 5.64 (t, J=6.1 Hz, 1H), 4.29-4.20 (m, 6H), 4.20-4.14 (m, 2H), 4.02 (dd, J=6.2, 1.4 Hz, 2H). MS (ESI⁺) calc. for C₃₂H₂₉IO₅P⁺ [M+H]⁺ 651.1, found 651.5.

Step 2: The intermediate (0.258 g, 0.397 mmol) was reacted according to general procedure XXH, to yield alkyl phosphate XS48 (76 mg, 49%) as the triethylamine salt in a 1:0.9 ratio of phosphate:Et₃N. ¹H NMR (400 MHz, DMSO-d₆) ppm=6.24 (tt, J=5.3, 1.4 Hz, 1H), 4.28-4.23 (m, 2H), 4.04 (d, J=1.6 Hz, 2H), 2.95 (q, J=7.1 Hz, 5H), 1.15 (t, J=7.3 Hz, 8H). MS (ESI⁻) calc, for C₄H₇IO₅P⁻ [M−H]⁻ 292.9, found 292.9.

### Preparation of 4-((14S,17S)-1-Azido-14-isopropyl-17-methyl-12, 15-dioxo-3,6,9-trioxa-13,16-diazaoctadecan-18-amido)benzyl dihydrogen phosphate (XD36)

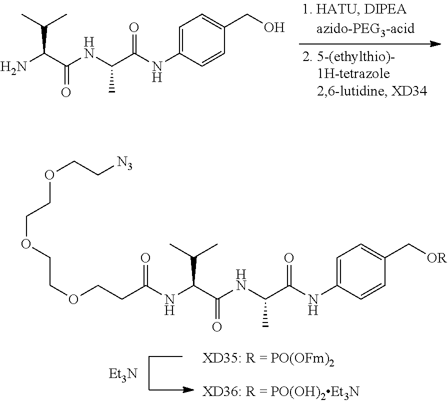

### bis((9H-Fluoren-9-yl)methyl) (4-((14S,17S)-1-azido-14-isopropyl-17-methyl-12,15-dioxo-3,6,9-trioxa-13,16-diazaoctadecan-18-amido)benzyl) phosphate (XD35)

Step 1:3-(2-(2-(2-azidoethoxy) ethoxy) ethoxy) propanoic acid (142 mg, 0.574 mmol) was dissolved in DMF (1 mL). Val-Ala-PAB-OH (160 mg, 0.545 mmol) in DMF (3.0 mL) was added, followed by the addition of HATU (228 mg, 0.600 mmol) and DIPEA (0.143 mL, 0.818 mmol) at RT. The reaction was stirred for 30 min before being concentrated. The crude was taken up in MeOH (1 mL) and basic impurities were removed by passing the solution through a short DOWEX 50WX8 plug that had been pre-washed with methanol. The product was eluted with methanol and the crude product was concentrated on silica gel. Purification by flash chromatography (silica gel, 0-8% MeOH in DCM) afforded the resulting amide (262 mg, 92%) as a cream solid. MS (ESI⁺) calcd, for C₂₄H₃₉N₆O₇⁺ [M+H]⁺ 523.3 found 523.6.

Step 2: To the amide product (977 mg, 1.87 mmol) and 5-(ethylthio)-1H-tetrazole (19 mg, 0.15 mmol) in MeCN (3.7 mL) under N₂ was added 2,6-lutidine (719 μL, 6.17 mmol) at RT followed by a solution of chloride XD34 (884 mg, 1.87 mmol) in DCM (3.7 mL), and the mixture was stirred at RT. More chloride XD34 was added after 80 min (88 mg, 0.187 mmol), and 140 min (177 mg, 0.374 mmol). After a total reaction time of 185 min, more 2,6-lutidine (218 μL, 1.87 mmol) was added and the reaction was continued for 2 h before being quenched with methanol (1 mL). The mixture was concentrated and the crude was taken up in EtOAc (80 mL) and aq. HCl (40 mL, 1 M). A small amount of MeCN (4 mL) was added to dissolve residual solids and the layers were separated. The water layer was extracted with EtOAc (80 mL) and the combined organic layers were washed with brine and dried over Na₂SO₄. The crude was purified by flash chromatography (silica gel, 0-5% MeOH in DCM) to yield phosphate ester XD35 (1.40 g, 66% yield). ¹H NMR (400 MHz, DMSO-d₆) ppm=9.94 (s, 1H), 8.18 (d, J=7.0 Hz, 1H), 7.89-7.82 (m, 5H), 7.55 (d, J=8.6 Hz, 2H), 7.52-7.44 (m, 4H), 7.42-7.34 (m, 4H), 7.30-7.24 (m, 4H), 7.09 (d, J=8.6 Hz, 2H), 4.60 (d, J=8.8 Hz, 2H), 4.40 (quint, J=7.0 Hz, 1H), 4.25-4.17 (m, 5H), 4.15-4.11 (m, 2H), 3.62-3.56 (m, 4H), 3.55-3.46 (m, 8H), 3.39-3.36 (m, 2H), 2.50-2.36 (m, 2H), 2.02-1.93 (m, 1H), 1.31 (d, J=7.1 Hz, 3H), 0.88 (d, J=6.8 Hz, 3H), 0.84 (d, J=6.8 Hz, 3H). MS (ESI⁺) calcd. for C₅₂H₅₉N₆O₁₀PNa⁺ [M+H]⁺ 981.4, found 981.8.

### 4-((14S,17S)-1-Azido-14-isopropyl-17-methyl-12,15-dioxo-3,6,9-trioxa-13,16-diazaoctadecan-18-amido)benzyl dihydrogen phosphate (XD36)

Triethylamine (0.25 mL) was added to a RT solution of phosphate XD35 (160 mg, 0.167 mmol) in MeCN (1 mL), and the reaction was stirred for 24 h. The reaction was diluted with toluene (8 mL) and then concentrated. The crude was suspended in ether (10 mL), filtered, and the solid was repetitively washed with ether to give alkyl phosphate XD36 (108 mg, 92%) as the mono triethylammonium salt. (Note: The product contained an impurity (m/z 606), potentially formed by elimination of the phosphate and trapping of the intermediate azaquinone methide with triethylamine. This impurity is unreactive in the next step and no further purification was required). MS (ESI⁻) calcd, for C₂₄H₃₈N₆O₁₀P⁻ [M−H]⁻ 601.2, found 601.7.

### Preparation of Alkyne Linker (XD43)

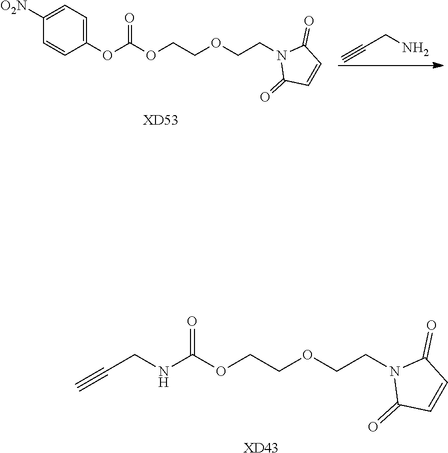

### 2-(2-(2,5-Dioxo-2,5-dihydro-1H-pyrrol-1-yl) ethoxy)ethyl prop-2-yn-1-ylcarbamate (XD43)

To PNP-carbonate XD53 (511 mg, 1.46 mmol, synthesized according to Elgersma, R. C. et al. Mol. Pharm. 2015, 12, 1813-1835) in THF (10 mL) at 0° C., propargylamine (0.093 mL, 1.46 mmol) was added. The cooling bath was removed and the mixture was stirred for 2 h at RT. The mixture was concentrated and the crude product was purified by flash chromatography (silica gel, 0-70% EtOAc in heptane), to give XD43 (265 mg, 68%) as a white solid. ¹H NMR (400 MHz, DMSO-d₆) ppm=7.60 (br t, J=5.5 Hz, 1H), 7.02 (s, 2H), 4.07-3.96 (m, 2H), 3.74 (dd, J=5.8, 2.4 Hz, 2H), 3.61-3.48 (m, 7H), 3.07 (t, J=2.5 Hz, 1H).

### 2D. Synthesis of Linker-Drugs XS54-XS58

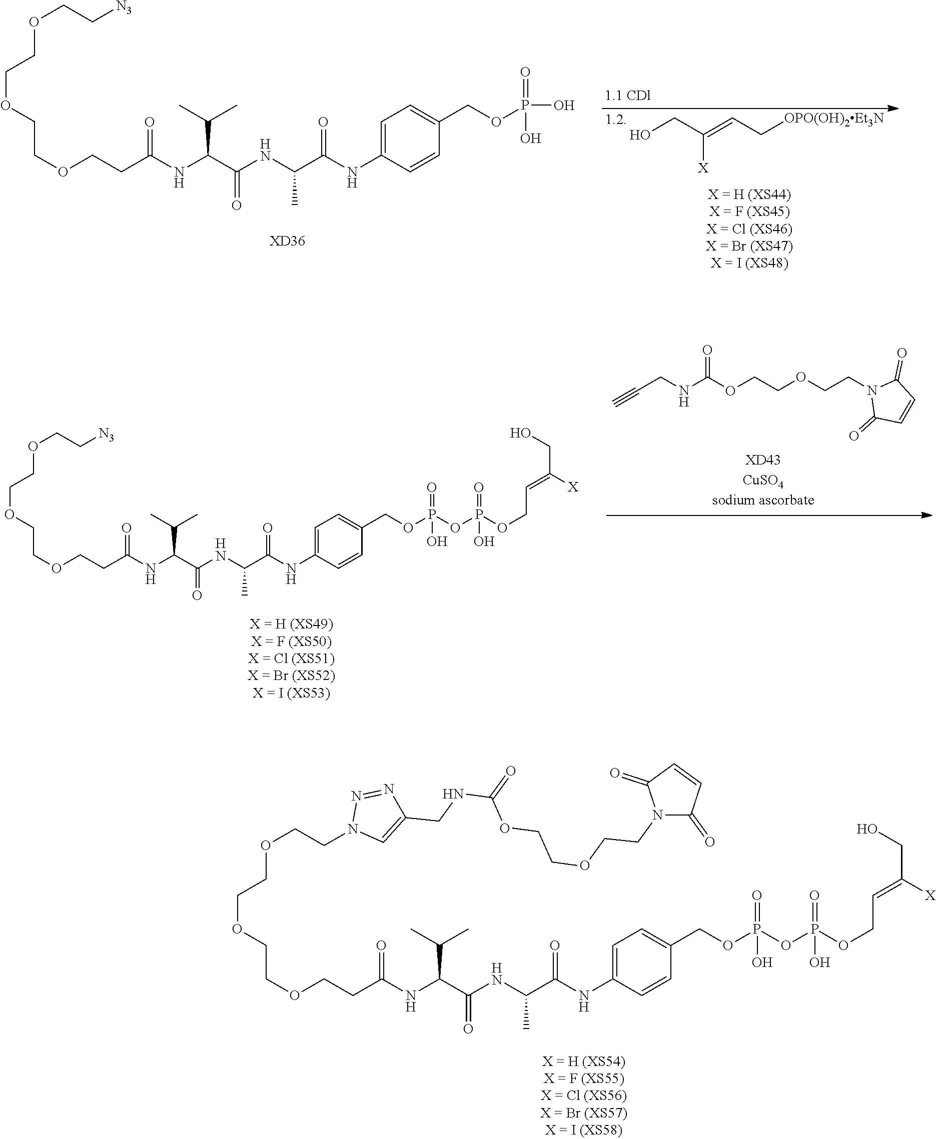

### Pyrophosphate XS49

Alkyl phosphate XD36 (32 mg, 0.045 mmol) was reacted with phosphate XS44 according to general procedure XXD. The crude was purified by preparative RP-HPLC (25 mM NH₄HCO₃ in MilliQ/MeCN, gradient 90:10 to 50:50), to give after lyophilization, pyrophosphate XS49 (19.6 mg, 55%) as a white solid. MS (ESI⁺) calc, for C₂₈H₄₇N₆O₁₄P2+ [M+H]⁺ 753.3, found 753.8.

### Pyrophosphate XS50

Alkyl phosphate XD36 (55 mg, 0.078 mmol) was reacted with phosphate XS45 according to general procedure XXD. The crude was purified by preparative RP-HPLC (25 mM NH₄HCO₃ in MilliQ/MeCN, gradient 90:10 to 60:40), to give after lyophilization, pyrophosphate XS50 (28.8 mg, 46%) as a white solid. ¹H NMR (400 MHz, D₂O)ppm=7.46-7.39 (m, 4H), 5.15 (dt, J=35.8, 7.2 Hz, 1H), 4.94 (d, J=6.8 Hz, 2H), 4.47 (t, J=7.3 Hz, 2H), 4.39 (q, J=7.2 Hz, 1H), 4.12 (d, J=7.1 Hz, 1H), 4.03 (d, J=15.9 Hz, 2H), 3.73 (t, J=6.0 Hz, 2H), 3.67-3.56 (m, 10H), 3.45-3.40 (m, 2H), 2.64-2.48 (m, 2H), 2.12-1.98 (m, 1H), 1.43 (d, J=7.3 Hz, 3H), 0.92 (d, J=5.1 Hz, 3H), 0.90 (d, J=5.1 Hz, 3H). MS (ESI⁺) calc, for C₂₈H₄₆FN₆O₁₄P₂⁺ [M+H]⁺ 771.3, found 771.9.

### Pyrophosphate XS51

Alkyl phosphate XD36 (100 mg, 0.142 mmol) was reacted with phosphate XS46 according to general procedure XXD. The crude was purified by preparative RP-HPLC (25 mM NH₄HCO₃ in MilliQ/MeCN, gradient 90:10 to 60:40), to give after lyophilization, pyrophosphate XS51 (62.4 mg, 54%) as a white solid. ¹H NMR (400 MHz, D₂O) ppm=7.36 (d, J=1.8 Hz, 4H), 5.90 (t, J=6.0 Hz, 1H), 4.86 (d, J=7.0 Hz, 2H), 4.50-4.42 (m, 2H), 4.37-4.28 (m, 1H), 4.09-4.03 (m, 1H), 4.01 (d, J=0.9 Hz, 2H), 3.66 (t, J=6.0 Hz, 2H), 3.59-3.49 (m, 10H), 3.39-3.33 (m, 2H), 2.57-2.41 (m, 2H), 1.98 (dq, J=13.7, 6.8 Hz, 1H), 1.36 (d, J=7.3 Hz, 3H), 0.85 (d, J=5.1 Hz, 3H), 0.84 (d, J=5.1 Hz, 3H). MS (ESI⁺) calc. for C₂₈H₄₆ClN₆O₁₄P₂⁺ [M+H]⁺ 787.2, found 787.6.

### Pyrophosphate XS52

Alkyl phosphate XD36 (70 mg, 0.099 mmol) was reacted with phosphate XS47 according to general procedure XXD. The crude was purified by preparative RP-HPLC (25 mM NH₄HCO₃ in MilliQ/MeCN, gradient 90:10 to 60:40), to give after lyophilization, pyrophosphate XS52 (51.9 mg, 60%) as a white solid. ¹H NMR (400 MHz, D₂O)ppm=7.45-7.36 (m, 4H), 6.17 (t, J=5.8 Hz, 1H), 4.95-4.88 (m, 2H), 4.46 (t, J=6.5 Hz, 2H), 4.41-4.34 (m, 1H), 4.15-4.06 (m, 3H), 3.71 (t, J=5.9 Hz, 2H), 3.65-3.53 (m, 10H), 3.45-3.37 (m, 2H), 2.65-2.42 (m, 2H), 2.13-1.95 (m, 1H), 1.41 (d, J=7.3 Hz, 3H), 0.90 (d, J=5.3 Hz, 3H), 0.89 (d, J=5.3 Hz, 3H). MS (ESI⁺) calc, for C₂₈H₄₆BrN₆O₁₄P₂⁺ [M+H]⁺ 831.2, found 831.6.

### Pyrophosphate XS53

Alkyl phosphate XD36 (100 mg, 0.142 mmol) was reacted with phosphate XS48 according to general procedure XXD. The crude was purified by preparative RP-HPLC (25 mM NH₄HCO₃ in MilliQ/MeCN, gradient 90:10 to 50:50), to give after lyophilization pyrophosphate XS53 (66.4 mg, 51%) as a white solid. ¹H NMR (400 MHz, D₂O) ppm=7.46-7.39 (m, 4H), 6.12 (t, J=5.6 Hz, 1H), 4.96-4.90 (m, 2H), 4.44-4.33 (m, 3H), 4.14-4.09 (m, 3H), 3.73 (t, J=6.0 Hz, 2H), 3.66-3.54 (m, 10H), 3.45-3.39 (m, 2H), 2.65-2.47 (m, 2H), 2.11-1.98 (m, 1H), 1.43 (d, J=7.1 Hz, 3H), 0.92 (d, J=5.4 Hz, 3H), 0.90 (d, J=5.5 Hz, 3H). MS (ESI⁺) calc, for C₂₈H₄₆IN₆O₁₄P₂⁺ [M+H]⁺ 879.2, found 879.6.

### Linker-Drug XS54

Azide XS49 (19.6 mg, 0.025 mmol) was reacted with alkyne XD43 according to general procedure XXE. Purification by preparative RP-HPLC (25 mM NH₄HCO₃ in MilliQ/MeCN, gradient 90:10 to 50:50), afforded after lyophilization pyrophosphate XS54 (11.7 mg, 45%) as a white solid. ¹H NMR (400 MHz, D₂O) ppm=7.92 (br s, 1H), 7.40 (s, 4H), 6.73 (s, 2H), 5.90-5.79 (m, 1H), 5.79-5.69 (m, 1H), 4.92 (s, 2H), 4.53 (t, J=4.8 Hz, 2H), 4.41-4.33 (m, 3H), 4.33-4.26 (m, 2H), 4.14-4.04 (m, 3H), 4.01 (d, J=4.9 Hz, 2H), 3.87 (t, J=4.9 Hz, 2H), 3.70 (t, J=5.9 Hz, 2H), 3.65-3.57 (m, 6H), 3.57-3.45 (m, 8H), 2.63-2.46 (m, 2H), 2.03 (dq, J=13.7, 6.8 Hz, 1H), 1.41 (d, J=7.1 Hz, 3H), 0.90 (d, J=6.9 Hz, 3H), 0.88 (d, J=6.9 Hz, 3H). MS (ESI⁺) calc, for C₄₀H₆₁N₈O₁₉P₂⁺ [M+H]⁺ 1019.4, found 1020.0.

### Linker-Drug XS55

Azide XS50 (24.5 mg, 0.030 mmol) was reacted with alkyne XD43 according to general procedure XXE. Purification by preparative RP-HPLC (25 mM NH₄HCO₃ in MilliQ/MeCN, gradient 90:10 to 60:40), afforded after lyophilization pyrophosphate XS55 (12.6 mg, 39%) as a white solid. ¹H NMR (400 MHz, D₂O) ppm=7.92 (br s, 1H), 7.47-7.35 (m, 4H), 6.74 (s, 2H), 5.15 (dt, J=35.8, 7.0 Hz, 1H), 4.99-4.88 (m, 2H), 4.53 (t, J=4.8 Hz, 2H), 4.50-4.45 (m, 2H), 4.38 (q, J=7.2 Hz, 1H), 4.31 (t, J=7.3 Hz, 2H), 4.15-4.06 (m, 3H), 4.03 (d, J=15.9 Hz, 2H), 3.88 (t, J=4.9 Hz, 2H), 3.70 (t, J=5.9 Hz, 2H), 3.66-3.59 (m, 6H), 3.56-3.47 (m, 8H), 2.62-2.47 (m, 2H), 2.04 (dq, J=13.7, 6.8 Hz, 1H), 1.42 (d, J=7.3 Hz, 3H), 0.90 (d, J=7.0 Hz, 3H), 0.88 (d, J=6.9 Hz, 3H). MS (ESI⁺) calc, for C₄₀H₆₀FN₈O₁₉P2+ [M+H]⁺ 1037.3, found 1037.8.

### Linker-Drug XS56

Azide XS51 (59.2 mg, 0.072 mmol) was reacted with alkyne XD43 according to general procedure XXE. Purification by preparative RP-HPLC (25 mM NH₄HCO₃ in MilliQ/MeCN, gradient 90:10 to 60:40), afforded after lyophilization pyrophosphate XS56 (43.4 mg, 55%) as a white solid. ¹H NMR (400 MHz, D₂O) ppm=7.93 (br s, 1H), 7.40 (s, 4H), 6.73 (s, 2H), 5.96 (t, J=5.1 Hz, 1H), 4.92 (br s, 2H), 4.57-4.48 (m, 4H), 4.37 (q, J=7.2 Hz, 1H), 4.30 (br s, 2H), 4.11 (d, J=7.0 Hz, 1H), 4.07 (s, 4H), 3.87 (t, J=4.8 Hz, 2H), 3.70 (t, J=5.9 Hz, 2H), 3.66-3.58 (m, 6H), 3.57-3.42 (m, 8H), 2.61-2.46 (m, 2H), 2.04 (dq, J=13.7, 6.8 Hz, 1H), 1.41 (d, J=7.3 Hz, 3H), 0.90 (d, J=6.9 Hz, 3H), 0.88 (d, J=6.9 Hz, 3H). MS (ESI⁺) calc, for C₄₀H₆₀ClN₈O₁₉P₂⁺ [M+H]⁺ 1053.3, found 1053.9.

### Linker-Drug XS57

Azide XS52 (40.7 mg, 0.047 mmol) was reacted with alkyne XD43 according to general procedure XXE. Purification by preparative RP-HPLC (25 mM NH₄HCO₃ in MilliQ/MeCN, gradient 90:10 to 60:40), afforded after lyophilization pyrophosphate XS57 (23.9 mg, 45%) as a white solid. ¹H NMR (400 MHz, D₂O) ppm=7.89 (br s, 1H), 7.39 (s, 4H), 6.72 (s, 2H), 6.17 (t, J=5.4 Hz, 1H), 4.91 (br s, 2H), 4.51 (t, J=4.9 Hz, 2H), 4.46 (br s, 2H), 4.36 (q, J=7.2 Hz, 1H), 4.29 (br s, 2H), 4.14-4.02 (m, 5H), 3.86 (t, J=4.9 Hz, 2H), 3.68 (t, J=5.9 Hz, 2H), 3.64-3.57 (m, 6H), 3.54-3.45 (m, 8H), 2.61-2.45 (m, 2H), 2.09-1.95 (m, 1H), 1.40 (d, J=7.3 Hz, 3H), 0.89 (d, J=7.0 Hz, 3H), 0.86 (d, J=6.9 Hz, 3H). MS (ESI⁺) calc. for C₄₀H₆₀BrN₈O₁₉P₂⁺ [M+H]⁺ 1097.3, found 1097.6.

### Linker-Drug XS58

Azide XS53 (62.6 mg, 0.069 mmol) was reacted with alkyne XD43 according to general procedure XXE. Purification by preparative RP-HPLC (25 mM NH₄HCO₃ in MilliQ/MeCN, gradient 90:10 to 60:40), afforded after lyophilization pyrophosphate XS58 (41.2 mg, 51%) as a white solid. ¹H NMR (400 MHz, D₂O) ppm=7.89 (br s, 1H), 7.39 (s, 4H), 6.72 (s, 2H), 6.11 (t, J=5.0 Hz, 1H), 4.91 (br s, 2H), 4.51 (t, J=4.8 Hz, 2H), 4.44-4.32 (m, 3H), 4.29 (br s, 2H), 4.15-4.02 (m, 5H), 3.86 (t, J=4.9 Hz, 2H), 3.68 (t, J=5.9 Hz, 2H), 3.64-3.57 (m, 6H), 3.55-3.44 (m, 8H), 2.52 (q, J=5.8 Hz, 2H), 2.02 (dq, J=13.7, 6.8 Hz, 1H), 1.40 (d, J=7.1 Hz, 3H), 0.89 (d, J=6.9 Hz, 3H), 0.86 (d, J=6.9 Hz, 3H). MS (ESI⁺) calc, for C₄₀H₆₀IN₈O₁₉P₂⁺ [M+H]⁺ 1145.2, found 1146.1.

### Example 3: Synthesis of Linker Drug Compounds XS100-XS107

### 3A. Preparation of Ether (XS60)

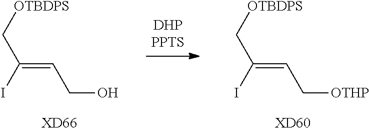

### (Z)-tert-butyl ((2-iodo-4-((tetrahydro-2H-pyran-2-yl)oxy)but-2-en-1-yl)oxy)diphenylsilane (XS60)

Alcohol XD66 (1.00 g, 2.21 mmol) was dissolved in DCM (2.2 mL) and PPTS (56 mg, 0.22 mmol) was added. The reaction mixture was cooled to 0° C. and DHP (303 μL, 3.32 mmol) was dropwise added. The reaction mixture was allowed to reach R^{T} and was stirred for 2 h. DCM was added and the mixture was washed with sat. aqueous NaHCO₃, water and brine, dried over Na₂SO₄ and concentrated. The crude product was purified by flash chromatography (silica gel, 0-10% EtOAc in heptane), to give ether XS60 (1.05 g, 89%) as a colorless oil. ¹H NMR (400 MHz, DMSO-d₆) ppm=7.64 (dd, J=7.8, 1.5 Hz, 4H), 7.52-7.41 (m, 6H), 6.30 (t, J=5.7 Hz, 1H), 4.60 (t, J=3.3 Hz, 1H), 4.30 (d, J=1.3 Hz, 2H), 4.24-4.01 (m, 2H), 3.75 (ddd, J=11.3, 8.3, 3.1 Hz, 1H), 3.49-3.41 (m, 1H), 1.76-1.57 (m, 2H), 1.55-1.39 (m, 4H), 1.03 (s, 9H). MS (ESI⁺) calcd, for C₂₅H₃₃INaO₃Si⁺ [M+Na]⁺ 559.1 found 559.5.

### 3B. Preparation of Functionalized Alkenes (XS61-XS65), Trifluoroalkene (XS71) and Alkyne (XS72)

### Preparation of Functionalized Alkenes (XS61-XS62)

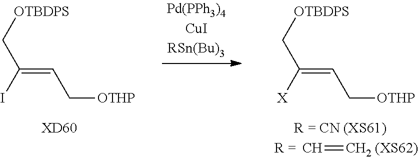

### (E)-2-(((tert-butyldiphenylsilyl)oxy)methyl)-4-((tetrahydro-2H-pyran-2-yl)oxy)but-2-enenitrile (XS61)

Tributylstannanecarbonitrile was reacted with XS60 (1.05 g, 1.96 mmol) according to general procedure XXI. The crude product was purified by flash chromatography (silica gel, 0-25% Et₂O in heptane) and filtered from heptane. The filtrate was concentrated to yield nitrile XS61 (0.882 g, quant.) as a yellow oil. ¹H NMR (400 MHz, CDCl₃) ppm=7.65 (dd, J=7.8, 1.4 Hz, 4H), 7.49-7.36 (m, 6H), 6.61-6.55 (m, 1H), 4.66 (t, J=3.5 Hz, 1H), 4.53-4.46 (m, 1H), 4.36-4.29 (m, 1H), 4.27-4.22 (m, 2H), 3.90-3.81 (m, 1H), 3.58-3.51 (m, 1H), 1.91-1.70 (m, 2H), 1.68-1.58 (m, 2H), 1.55-1.50 (m, 1H), 1.43-1.29 (m, 1H), 1.08 (s, 9H). MS (ESI⁺) calcd. for C₂₆H₃₄NO₃Si⁺ [M+H]⁺ 436.2 found 436.4.

### (E)-tert-butyldiphenyl ((4-((tetrahydro-2H-pyran-2-yl)oxy)-2-vinylbut-2-en-1-yl)oxy) silane (XS62)

Tributyl (vinyl) stannane was reacted with XS60 (0.400 g, 0.746 mmol) according to general procedure XXI. The crude product was purified by flash chromatography (silica gel, 0-10% EtOAc in heptane) to yield diene XS62 (0.284 g, 87%) as a yellow solid. ¹H NMR (400 MHz, DMSO-d₆) ppm=7.66-7.61 (m, 4H), 7.50-7.41 (m, 6H), 6.60 (dd, J=17.7, 11.3 Hz, 1H), 5.87 (t, J=6.7 Hz, 1H), 5.22-5.09 (m, 2H), 4.59 (t, J=3.5 Hz, 1H), 4.36 (s, 2H), 4.33-4.18 (m, 2H), 3.75 (ddd, J=11.2, 8.1, 3.1 Hz, 1H), 3.49-3.38 (m, 1H), 1.76-1.56 (m, 2H), 1.55-1.39 (m, 4H), 1.01 (s, 9H). MS (ESI⁺) calcd, for C₂₇H₃₇O₃Si⁺ [M+H]⁺ 437.3 found 437.4.

Preparation of functionalized alkene (XS63)

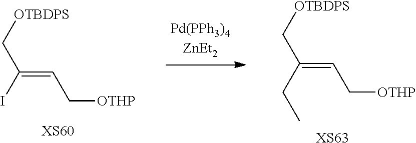

### (E)-tert-butyl ((2-ethyl-4-((tetrahydro-2H-pyran-2-yl)oxy)but-2-en-1-yl)oxy)diphenylsilane (XS63)

Diethylzinc (1.0 M in hexanes, 1.92 mL) was reacted with XS60 (0.515 g, 0.960 mmol) according to general procedure XXJ. The crude product was purified by flash chromatography (silica gel, 0-10% EtOAc in heptane) to yield alkane XS63 (0.193 g, 46%) as a colorless oil. ¹H NMR (400 MHz, CDCl₃) ppm=7.70-7.66 (m, 4H), 7.45-7.34 (m, 6H), 5.69 (t, J=6.8 Hz, 1H), 4.64 (t, J=3.6 Hz, 1H), 4.29 (dd, J=12.3, 6.2 Hz, 1H), 4.17-4.08 (m, 3H), 3.93-3.86 (m, 1H), 3.56-3.47 (m, 1H), 2.15-2.00 (m, 2H), 1.91-1.68 (m, 2H), 1.66-1.57 (m, 2H), 1.56-1.50 (m, 2H), 1.06 (s, 9H), 0.92 (t, J=7.6 Hz, 3H). MS (ESI⁺) calcd, for C₂₇H₃₈NaO₃Si⁺ [M+H]⁺461.2 found 461.4.

### Functionalized Alkene (XS64)

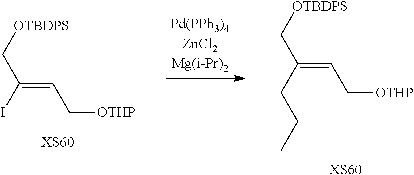

### (E)-tert-butyldiphenyl ((2-(2-((tetrahydro-2H-pyran-2-yl)oxy)ethylidene) pentyl)oxy) silane (XS64)

Isopropylzinc chloride (0.815 g, 5.74 mmol) (freshly prepared by adding i-PrMgCl (2.0 M in THF, 2.87 mL) to a solution of ZnCl₂ (0.782 g, 5.74 mmol) in THF (4.8 mL) at 0° C.) was reacted with XS60 (0.770 g, 1.44 mmol) according to procedure XXJ. The crude product was purified by flash chromatography (silica gel, 0-8% EtOAc in heptane) to yield alkane XS64 (0.335 g, 52%) as a colorless oil ¹H NMR (400 MHz, CDCl₃) ppm=7.70-7.66 (m, 4H), 7.44-7.33 (m, 6H), 5.74 (t, J=6.9 Hz, 1H), 4.67-4.62 (m, 1H), 4.28 (dd, J=12.2, 6.2 Hz, 1H), 4.17-4.12 (m, 1H), 4.11 (s, 2H), 3.94-3.85 (m, 1H), 3.55-3.48 (m, 1H), 2.10-1.96 (m, 2H), 1.90-1.68 (m, 2H), 1.64-1.56 (m, 2H), 1.54-1.49 (m, 2H), 0.36-1.28 (m, 2H), 1.06 (s, 9H), 0.83 (t, J=7.4

Functionalized alkene (XS65)

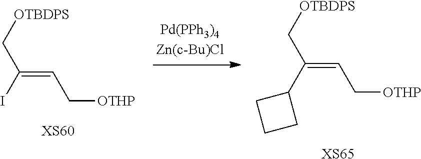

### (E)-tert-butyl ((2-cyclobutyl-4-((tetrahydro-2H-pyran-2-yl)oxy)but-2-en-1-yl)oxy)diphenylsilane (XS65)

Cyclobutylzinc chloride (0.5 M in THF, 11.2 mL) was reacted with XS60 (0.750 g, 1.40 mmol) according to general procedure XXJ. The crude product was purified by flash chromatography (silica gel, 0-10% EtOAc in heptane) to yield cycloalkane XS65 (0.574 g, 88%) as a colorless oil. ¹H NMR (400 MHz, CDCl₃) ppm=7.72-7.67 (m, 4H), 7.46-7.34 (m, 6H), 5.67-5.61 (m, 1H), 4.65-4.60 (m, 1H), 4.25 (dd, J=12.4, 6.2 Hz, 1H), 4.21 (s, 2H), 4.11 (dd, J=12.3, 7.5 Hz, 1H), 3.94-3.86 (m, 1H), 3.55-3.48 (m, 1H), 3.33 (quint, J=9.1 Hz, 1H), 2.06-1.95 (m, 4H), 1.90-1.79 (m, 2H), 1.77-1.64 (m, 2H), 1.64-1.53 (m, 4H), 1.06 (s, 9H). MS (ESI⁺) calcd, for C₂₉H₄₀NaO₃Si⁺ [M+H]⁺ 487.3 found 487.5.

### Trifluoroalkene (XS71)

### Aldehyde (XS67)

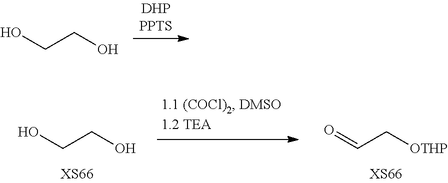

### 2-((tetrahydro-2H-pyran-2-yl)oxy) ethan-1-ol (XS66)

Ethylene glycol (5.00 mL, 90.0 mmol) was suspended in THF (90 mL) and cooled to 0° C. PTSA monohydrate (0.426 g, 2.24 mmol) was added, followed by dropwise addition of DHP (2.05 mL, 22.4 mmol). The reaction mixture was stirred at 0° C. for 30 min, allowed to reach R^{T} and stirred for 3 days. The reaction mixture was diluted with water and the product was extracted with DCM (2×). The organic layer was washed with brine, dried over Na₂SO₄ and concentrated. The crude product was purified by flash chromatography (silica gel, 0-60% EtOAc in heptane) to yield ether XS66 (2.24 g, 68%) as a colorless oil. ¹H NMR (400 MHz, CDCl₃) ppm=4.60-4.55 (m, 1H), 3.98-3.90 (m, 1H), 3.82-3.66 (m, 4H), 3.61-3.50 (m, 1H), 2.85 (dd, J=7.0, 4.5 Hz, 1H), 1.88-1.72 (m, 2H), 1.64-1.50 (m, 4H). MS (ESI⁺) calcd. for C₇H₁₅O₃⁺ [M+H]⁺ 147.1 found 147.0.

### 2-((tetrahydro-2H-pyran-2-yl)oxy) acetaldehyde (XS67)

To a −78° C. solution of oxalyl chloride (1.74 mL, 19.9 mmol) in DCM (140 mL) was dropwise added DMSO (2.80 mL, 39.4 mmol). The reaction mixture was stirred for 1 h at −78° C., after which a solution of alcohol XS66 (2.24 g, 15.3 mmol) in DCM (13 mL) was dropwise added. The reaction mixture was stirred at −78° C. for 30 min, after which TEA (13.7 mL, 98.0 mmol) was dropwise added. The reaction mixture was stirred at −78° C. for 45 min, allowed to reach RT and stirred for 45 min. Sat. aqueous NaHCO₃ was added and the mixture was stirred for 15 min. The organic layer was separated and the water layer was extracted with DCM (2×). The combined organic layers were washed with brine, dried over Na₂SO₄ and concentrated. The crude product was purified by flash chromatography (silica gel, 0-50% EtOAc in heptane) to yield aldehyde XS67 (1.32 g, 60%) as a colorless oil. ¹H NMR (400 MHz, CDCl₃) ppm=9.76 (t, J=1.0 Hz, 1H), 4.66 (dd, J=4.0, 3.1 Hz, 1H), 4.24 (dd, J=18.0, 1.2 Hz, 1H), 4.17 (dd, J=18.0, 0.8 Hz, 1H), 3.92-3.82 (m, 1H), 3.59-3.48 (m, 1H), 1.91-1.67 (m, 4H), 1.58 (td, J=4.2, 2.5 Hz, 2H).

### Trifluoroalkene (XS71)

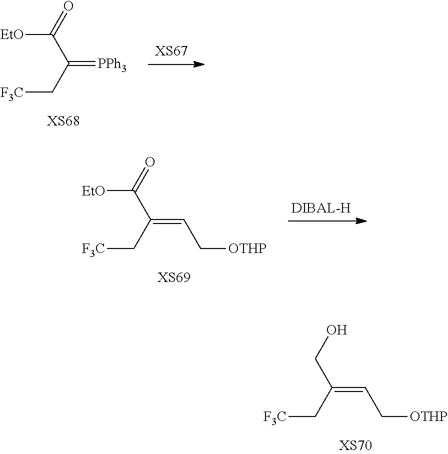

### ethyl (E)-4-((tetrahydro-2H-pyran-2-yl)oxy)-2-(2,2,2-trifluoroethyl)but-2-enoate (XS69)

To a solution of aldehyde XS67 (0.894 g, 6.20 mmol) in THF (4.1 mL) was added a solution of Wittig reagent XS68 (1.78 g, 4.14 mmol, synthesized according to US2007/249723) in THF (4.1 mL). The reaction mixture was stirred for 16 h at 100° C. The reaction mixture was diluted with Et₂O, dried over Na₂SO₄ and filtered over a silica plug. The filtrate was concentrated and the crude product was purified by flash chromatography (silica gel, 0-20% EtOAc in heptane) to yield E-alkene XS69 (0.435 g, 36%) as a colorless oil. ¹H NMR (400 MHz, CDCl₃) ppm=7.20 (t, J=6.0 Hz, 1H), 4.65 (t, J=3.3 Hz, 1H), 4.47 (dd, J=15.1, 5.7 Hz, 1H), 4.29-4.19 (m, 3H), 3.85 (ddd, J=11.3, 8.4, 3.2 Hz, 1H), 3.58-3.51 (m, 1H), 3.28 (qd, J=10.5, 1.8 Hz, 2H), 1.89-1.70 (m, 2H), 1.67-1.50 (m, 4H), 1.32 (t, J=7.1 Hz, 3H).

### (E)-4-((tetrahydro-2H-pyran-2-yl)oxy)-2-(2,2,2-trifluoroethyl)but-2-en-1-ol (XS70)

Ester XS69 (0.425 g, 1.43 mmol) was purged with N₂ and dissolved in THF (7.2 mL). At 0° C., DIBAL-H (1.0 M in hexane, 3.16 mL) was dropwise added. The reaction mixture was stirred at 0° C. for 2 h. Next, HCl (1.0 M) was added and the reaction mixture was stirred for 30 min. The aqueous layer was extracted with DCM (3×) and the combined organic layers were washed with brine, dried over Na₂SO₄ and concentrated. The crude product was purified by flash chromatography (silica gel, 0-40% EtOAc in heptane) to yield alcohol XS70 (0.232 g, 64%) as a pale yellow oil. ¹H NMR (400 MHz, CDCl₃) ppm=6.00 (t, J=6.4 Hz, 1H), 4.63 (t, J=3.4 Hz, 1H), 4.33 (dd, J=13.0, 6.1 Hz, 1H), 4.17 (d, J=6.4 Hz, 2H), 4.09 (dd, J=13.1, 6.9 Hz, 1H), 3.86 (ddd, J=11.2, 8.0, 3.4 Hz, 1H), 3.57-3.49 (m, 1H), 3.07-2.93 (m, 2H), 1.86-1.71 (m, 2H), 1.63-1.57 (m, 2H), 1.54-1.51 (m, 2H), 1.51-1.45 (m, 1H).

### (E)-tert-butyldiphenyl ((4-((tetrahydro-2H-pyran-2-yl)oxy)-2-(2,2,2-trifluoroethyl)but-2-en-1-yl)oxy) silane (XS71)

To a 0° C. solution of alcohol XS70 (0.230 g, 0.905 mmol), TEA (0.252 mL, 1.81 mmol) and imidazole (68 mg, 1.0 mmol) was dropwise added TBDPSCl (0.349 mL, 1.36 mmol). The reaction mixture was allowed to reach R^{T} and was stirred for 3 h. Water was added and the product was extracted with DCM (3×). The combined organic layers were washed with brine, dried over Na₂SO₄ and concentrated. The crude product was purified by flash chromatography (silica gel, 0-10% EtOAc in heptane) to yield silyl ether XS71 (0.378 g, 85%) as a colorless oil. ¹H NMR (400 MHz, CDCl₃) ppm=7.68-7.63 (m, 4H), 7.45-7.35 (m, 6H), 5.99 (t, J=6.5 Hz, 1H), 4.62 (t, J=3.4 Hz, 1H), 4.33-4.27 (m, 1H), 4.18-4.16 (m, 2H), 4.12 (dd, J=13.1, 7.3 Hz, 1H), 3.90-3.82 (m, 1H), 3.55-3.49 (m, 1H), 3.06-2.85 (m, 2H), 1.90-1.67 (m, 2H), 1.66-1.54 (m, 4H), 1.06 (s, 9H).

### Alkyne (XS72)

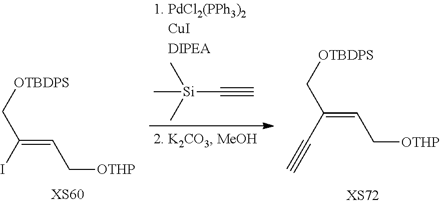

### (E)-tert-butyldiphenyl ((4-((tetrahydro-2H-pyran-2-yl)oxy)-2-((trimethylsilyl) ethynyl)but-2-en-1-yl)oxy) silane (XS72)

CuI (16 mg, 0.085 mmol) and Pd(PPh₃) 2Cl₂ (60 mg, 0.085 mmol) were purged with N₂ (3×) and a solution of XS60 (0.458 g, 0.854 mmol) in toluene (5.0 mL) was added, followed by trimethylsilylacetylene (0.126 g, 1.28 mmol) and DIPEA (0.298 mL, 1.71 mmol). The reaction mixture was stirred at RT for 6 h, concentrated and taken up in EtOAc. The organic layer was washed with 0.5 M aqueous KHSO₄, water and brine, dried over Na₂SO₄ and concentrated. The crude residue was purified by flash chromatography (silica gel, 0-8% EtOAc in heptane) and filtered from heptane. The filtrate was concentrated to yield the silyl ether intermediate (0.361 g, 84%) as an orange oil. The intermediate was dissolved in MeOH (7.1 mL) and K₂CO₃ (49 mg, 0.36 mmol) was added. The reaction mixture was stirred at RT for 2 h. The reaction mixture was diluted with EtOAc and washed with sat. aqueous NaHCO₃, water and brine, dried over Na₂SO₄ and concentrated. The crude product was purified by flash chromatography (silica gel, 0-10% EtOAc in heptane) to yield alkyne XS72 (0.192 g, 62%) as a colorless oil. ¹H NMR (400 MHz, CDCl₃) ppm=7.69-7.65 (m, 4H), 7.46-7.34 (m, 6H), 6.40-6.32 (m, 1H), 4.66 (t, J=3.6 Hz, 1H), 4.52-4.43 (m, 1H), 4.41-4.32 (m, 1H), 4.20 (d, J=1.4 Hz, 2H), 3.95-3.86 (m, 1H), 3.57-3.49 (m, 1H), 3.12 (s, 1H), 1.92-1.69 (m, 2H), 1.67-1.56 (m, 2H), 1.54-1.49 (m, 2H), 1.07 (s, 9H).

### 3C. Preparation of Phosphates (XS87-XS93)

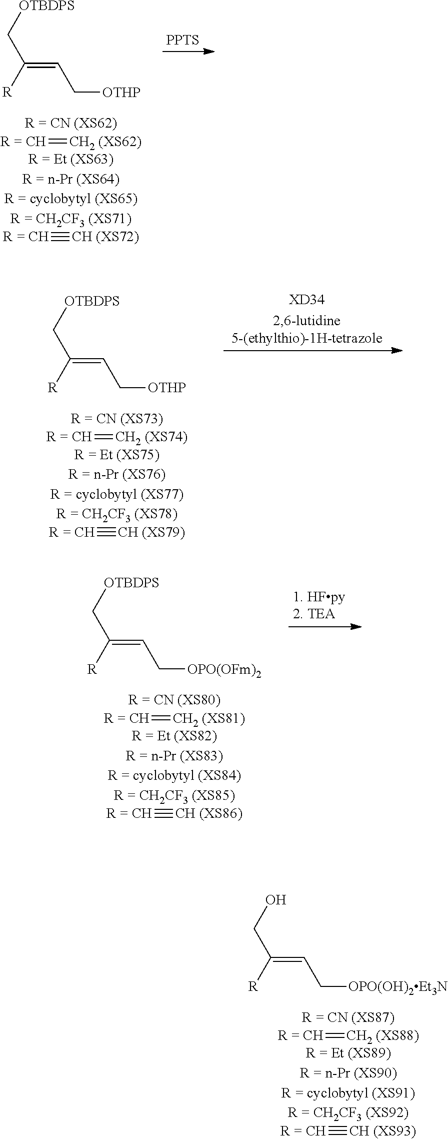

### (E)-2-(((tert-butyldiphenylsilyl)oxy)methyl)-4-hydroxybut-2-enenitrile (XS73)

Ether XS61 (0.500 g, 1.15 mmol) was reacted according to procedure XXK at 45° C. Instead of DCM, Et₂O was used for the extraction. The crude product was purified by flash chromatography (silica gel, 0-60% Et₂O in heptane) to yield alcohol XS73 (0.261 g, 65%) as a pale yellow oil. ¹H NMR (400 MHz, DMSO-d₆) ppm=7.64 (dd, J=7.8, 1.7 Hz, 4H), 7.54-7.41 (m, 6H), 6.62-6.53 (m, 1H), 5.29 (t, J=5.7 Hz, 1H), 4.31 (d, J=1.3 Hz, 2H), 4.19 (t, J=5.9 Hz, 2H), 1.02 (s, 9H). MS (ESI⁺) calcd, for C₂₁H₂₆NO₂Si⁺ [M+H]⁺ 352.2 found 352.2.

### (E)-3-(((tert-butyldiphenylsilyl)oxy)methyl) penta-2,4-dien-1-ol (XS74)

Ether XS62 (0.284 g, 0.650 mmol) was reacted according to procedure XXK at 55° C. The crude product was purified by flash chromatography (silica gel, 0-50% Et₂O in heptane) to yield alcohol XS74 (0.183 g, 80%) as a pale yellow oil and used as such in the next reaction step.

### (E)-3-(((tert-butyldiphenylsilyl)oxy)methyl) pent-2-en-1-ol (XS75)

Ether XS63 (0.193 g, 0.440 mmol) was reacted according to procedure XXK at 55° C. The crude product was purified by flash chromatography (silica gel, 0-50% Et₂O in heptane) to yield alcohol XS75 (0.112 g, 72%) as a colorless oil. ¹H NMR (400 MHz, CDCl₃) ppm=7.68 (dd, J=7.8, 1.4 Hz, 4H), 7.46-7.35 (m, 6H), 5.71 (t, J=6.9 Hz, 1H), 4.23-4.18 (m, 2H), 4.12 (s, 2H), 2.05 (q, J=7.5 Hz, 2H), 1.26 (s, 1H), 1.07 (s, 9H), 0.92 (t, J=7.6 Hz, 3H). MS (ESI⁺) calcd, for C₂₂H₃₀NaO₂Si⁺ [M+Na]⁺ 377.2 found 377.3.

### (E)-3-(((tert-butyldiphenylsilyl)oxy)methyl) hex-2-en-1-ol (XS76)

Ether XS64 (0.330 g, 0.729 mmol) was reacted according to procedure XXK at 55° C. The crude product was purified by flash chromatography (silica gel, 0-40% Et₂O in heptane) to yield alcohol XS76 (0.241 g, 90%) as a colorless oil. ¹H NMR (400 MHz, CDCl₃) ppm=7.71-7.64 (m, 4H), 7.46-7.33 (m, 6H), 5.77 (t, J=7.0 Hz, 1H), 4.23-4.16 (m, 2H), 4.11 (s, 2H), 2.04-1.98 (m, 2H), 1.38-1.24 (m, 3H), 1.07 (s, 9H), 0.83 (t, J=7.4 Hz, 3H). MS (ESI⁺) calcd, for C₂₃H₃₁₀Si⁺ [M+H−H₂O]⁺ 351.2 found 351.4.

### (E)-4-((tert-butyldiphenylsilyl)oxy)-3-cyclobutylbut-2-en-1-ol (XS77)

Ether XS65 (0.570 g, 1.23 mmol) was reacted according to procedure XXK at 50° C. The crude product was purified by flash chromatography (silica gel, 0-50% Et₂O in heptane) to yield alcohol XS77 (0.414 g, 89%) as a colorless oil. ¹H NMR (400 MHz, CDCl₃) ppm=7.69 (dd, J=7.8, 1.6 Hz, 4H), 7.46-7.36 (m, 6H), 5.66 (td, J=6.8, 1.3 Hz, 1H), 4.22-4.16 (m, 4H), 3.30 (quint, J=9.0 Hz, 1H), 2.03-1.96 (m, 4H), 1.92-1.79 (m, 1H), 1.72-1.63 (m, 1H), 1.26 (s, 1H), 1.07 (s, 9H). MS (ESI⁺) calcd, for C₂₄H₃₂NaO₂Si⁺ [M+Na]⁺ 403.2 found 403.3.

### (E)-3-(((tert-butyldiphenylsilyl)oxy)methyl)-5,5,5-trifluoropent-2-en-1-ol (XS78)

Ether XS71 (0.375 g, 0.761 mmol) was reacted according to procedure XXK at 55° C. The crude product was purified by flash chromatography (silica gel, 0-40% Et₂O in heptane) to yield alcohol XS78 (0.272 g, 87%) as a colorless oil. ¹H NMR (400 MHz, CDCl₃) ppm=7.68-7.63 (m, 4H), 7.46-7.36 (m, 6H), 5.98 (t, J=6.8 Hz, 1H), 4.22 (t, J=6.3 Hz, 2H), 4.17 (s, 2H), 2.92 (q, J=11.0 Hz, 2H), 1.23 (t, J=5.8 Hz, 1H), 1.07 (s, 9H).

### (E)-3-(((tert-butyldiphenylsilyl)oxy)methyl) pent-2-en-4-yn-1-ol (XS79)

Ether XS72 (0.190 g, 0.437 mmol) was reacted according to procedure XXK at 55° C. The crude product was purified by flash chromatography (silica gel, 0-50% Et₂O in heptane) to yield alcohol XS79 (0.123 g, 80%) as a pale yellow oil. ¹H NMR (400 MHz, CDCl₃) ppm=7.70-7.65 (m, 4H), 7.46-7.36 (m, 6H), 6.36 (tt, J=6.6, 1.6 Hz, 1H), 4.45-4.39 (m, 2H), 4.20 (d, J=1.4 Hz, 2H), 3.14 (s, 1H), 1.43 (t, J=6.1 Hz, 1H), 1.08 (s, 9H). MS (ESI⁺) calcd. for C₂₂H₂₇O₂Si⁺ [M+H]⁺ 351.2 found 351.3.

### (E)-bis((9H-fluoren-9-yl)methyl) (4-((tert-butyldiphenylsilyl)oxy)-3-cyanobut-2-en-1-yl) phosphate (XS80)

Alcohol XS73 (0.261 g, 0.742 mmol) was reacted according to procedure XXL. The crude product was purified by flash chromatography (silica gel, 0-50% EtOAc in heptane) to yield alkyl phosphate XS80 (0.339 g, 58%) as a colorless oil. ¹H NMR (400 MHz, DMSO-d₆) ppm=7.83 (dd, J=7.5, 3.5 Hz, 4H), 7.66-7.57 (m, 4H), 7.57-7.32 (m, 14H), 7.30-7.23 (m, 4H), 6.34 (t, J=6.3 Hz, 1H), 4.40 (dd, J=9.1, 6.3 Hz, 2H), 4.34-4.23 (m, 6H), 4.20-4.14 (m, 2H), 0.98 (s, 9H). MS (ESI⁺) calcd, for C₄₉H₄₇NO₅PSi⁺ [M+H]⁺ 788.3 found 788.5.

### (E)-bis((9H-fluoren-9-yl)methyl) (3-(((tert-butyldiphenylsilyl)oxy)methyl) penta-2,4-dien-1-yl) phosphate (XS81)

Alcohol XS74 (0.183 g, 0.519 mmol) was reacted according to procedure XXL. The crude product was purified by flash chromatography (silica gel, 0-30% EtOAc in heptane) to yield alkyl phosphate XS81 (0.312 g, 76%) as a colorless oil. ¹H NMR (400 MHz, CDCl₃) ppm=7.71 (t, J=7.3 Hz, 4H), 7.65-7.59 (m, 4H), 7.52 (dd, J=15.6, 7.5 Hz, 4H), 7.41-7.29 (m, 10H), 7.26-7.21 (m, 4H), 6.41 (dd, J=17.9, 11.1 Hz, 1H), 5.90 (br t, J=6.9 Hz, 1H), 5.15-5.09 (m, 2H), 4.63 (t, J=7.4 Hz, 2H), 4.30-4.22 (m, 6H), 4.15 (t, J=6.6 Hz, 2H), 1.01 (s, 9H).

### (E)-bis((9H-fluoren-9-yl)methyl) (3-(((tert-butyldiphenylsilyl)oxy)methyl) pent-2-en-1-yl) phosphate (XS82)

Alcohol XS75 (0.110 g, 0.310 mmol) was reacted according to procedure XXL. The crude product was purified by flash chromatography (silica gel, 0-50% EtOAc in heptane) to yield alkyl phosphate XS82 (0.195 g, 79%) as a colorless oil. ¹H NMR (400 MHz, CDCl₃) ppm=7.71 (t, J=7.1 Hz, 4H), 7.62 (dd, J=7.9, 1.4 Hz, 4H), 7.57-7.48 (m, 4H), 7.42-7.30 (m, 10H), 7.28-7.20 (m, 4H), 5.67 (t, J=7.1 Hz, 1H), 4.52 (t, J=7.4 Hz, 2H), 4.26 (t, J=6.5 Hz, 4H), 4.20-4.11 (m, 2H), 4.06 (s, 2H), 1.96 (q, J=7.7 Hz, 2H), 1.01 (s, 9H), 0.87-0.82 (m, 3H). MS (ESI⁺) calcd, for C₅₀H₅₁NaO₅PSi⁺ [M+Na]⁺ 813.3 found 813.5.

### (E)-bis((9H-fluoren-9-yl)methyl) (3-(((tert-butyldiphenylsilyl)oxy)methyl) hex-2-en-1-yl) phosphate (XS83)

Alcohol XS76 (0.239 g, 0.648 mmol) was reacted according to procedure XXL. The crude product was purified by flash chromatography (silica gel, 0-40% EtOAc in heptane) to yield alkyl phosphate XS83 (0.347 g, 67%) as a colorless oil. ¹H NMR (400 MHz, CDCl₃) ppm=7.71 (t, J=7.1 Hz, 4H), 7.62 (dd, J=7.9, 1.4 Hz, 4H), 7.53 (dd, J=15.5, 7.5 Hz, 4H), 7.42-7.29 (m, 10H), 7.25-7.21 (m, 4H), 5.73 (t, J=7.1 Hz, 1H), 4.54 (t, J=7.4 Hz, 2H), 4.25 (t, J=6.5 Hz, 4H), 4.21-4.11 (m, 2H), 4.04 (s, 2H), 1.96-1.90 (m, 2H), 1.29-1.20 (m, 2H), 1.01 (s, 9H), 0.77 (t, J=7.3 Hz, 3H).

### (E)-bis((9H-fluoren-9-yl)methyl) (4-((tert-butyldiphenylsilyl)oxy)-3-cyclobutylbut-2-en-1-yl) phosphate (XS84)

Alcohol XS77 (0.410 g, 1.08 mmol) was reacted according to procedure XXL. The crude product was purified by flash chromatography (silica gel, 0-50% EtOAc in heptane) to yield alkyl phosphate XS84 (0.519 g, 59%) as a colorless oil. ¹H NMR (400 MHz, CDCl₃) ppm=7.71 (t, J=7.3 Hz, 4H), 7.63 (dd, J=7.9, 1.4 Hz, 4H), 7.57-7.49 (m, 4H), 7.42-7.30 (m, 10H), 7.25-7.20 (m, 4H), 5.63 (td, J=7.0, 1.3 Hz, 1H), 4.51 (t, J=7.3 Hz, 2H), 4.29-4.21 (m, 4H), 4.19-4.11 (m, 4H), 3.19 (quint, J=8.8 Hz, 1H), 1.96-1.87 (m, 4H), 1.87-1.74 (m, 1H), 1.68-1.58 (m, 1H), 1.01 (s, 9H).

### (E)-bis((9H-fluoren-9-yl)methyl) (3-(((tert-butyldiphenylsilyl)oxy)methyl)-5,5,5-trifluoropent-2-en-1-yl) phosphate (XS85)

Alcohol XS78 (0.135 g, 0.330 mmol) was reacted according to procedure XXL. The crude product was purified by flash chromatography (silica gel, 0-50% EtOAc in heptane) to yield alkyl phosphate XS85 (0.179 g, 64%) as a colorless oil. ¹H NMR (400 MHz, CDCl₃) ppm=7.71 (t, J=7.3 Hz, 4H), 7.62-7.56 (m, 4H), 7.55-7.45 (m, 4H), 7.42-7.30 (m, 10H), 7.29-7.19 (m, 4H), 5.91 (t, J=6.8 Hz, 1H), 4.43 (t, J=7.5 Hz, 2H), 4.29-4.22 (m, 4H), 4.17-4.11 (m, 2H), 4.11-4.06 (m, 2H), 2.78 (q, J=10.9 Hz, 2H), 1.01 (s, 9H).

### (E)-bis((9H-fluoren-9-yl)methyl) (3-(((tert-butyldiphenylsilyl)oxy)methyl) pent-2-en-4-yn-1-yl) phosphate (XS86)

Alcohol XS79 (0.120 g, 0.342 mmol) was reacted according to procedure XXL. The crude product was purified by flash chromatography (silica gel, 0-40% EtOAc in heptane) to yield alkyl phosphate XS86 (0.199 g, 74%) as a colorless oil. ¹H NMR (400 MHz, CDCl₃) ppm=7.71 (t, J=7.4 Hz, 4H), 7.62 (dd, J=7.9, 1.3 Hz, 4H), 7.53 (dd, J=14.3, 7.5 Hz, 4H), 7.43-7.29 (m, 10H), 7.29-7.21 (m, 4H), 6.28 (t, J=6.8 Hz, 1H), 4.77 (t, J=7.4 Hz, 2H), 4.32-4.23 (m, 4H), 4.20-4.12 (m, 4H), 3.09 (s, 1H), 1.03 (s, 9H). MS (ESI⁺) calcd. for C₅₀H₄₈O₅PSi⁺ [M+H]⁺ 787.3 found 787.6.

### Triethylamine (E)-3-cyano-4-hydroxybut-2-en-1-yl phosphate (XS87)

Step 1: TBDPS-ether XS80 (0.339 g, 0.430 mmol) was reacted according to general procedure XXG. The crude intermediate was purified by flash chromatography (silica gel, 0-100% EtOAc in heptane) to yield the intermediate allylic alcohol (0.164 g, 69%) as a white solid. MS (ESI⁺) calc, for C₃₃H₂₉NO₅P⁺ [M+H]⁺ 550.2, found 550.3.

Step 2: The intermediate (0.164 g, 0.298 mmol) was reacted according to general procedure XXH, to yield alkyl phosphate XS87 (79 mg, 94%) as the triethylamine salt in a 1:0.9 ratio of phosphate:Et₃N. ¹H NMR (400 MHz, D₂O) ppm=6.57 (t, J=6.1 Hz, 1H), 4.57-4.50 (m, 2H), 4.12 (d, J=0.9 Hz, 2H). MS (ESI) calc, for C₅H₇NO₇P⁻ [M−H]⁻ 192.0, found 192.0.

### Triethylamine (E)-3-(hydroxymethyl) penta-2,4-dien-1-yl phosphate (XS88)

Step 1: TBDPS-ether XS81 (0.305 g, 0.387 mmol) was reacted according to general procedure XXG. The crude intermediate was purified by flash chromatography (silica gel, 0-100% EtOAc in heptane) to yield the intermediate allylic alcohol (0.172 g, 81%) as a white solid. ¹H NMR (400 MHz, CDCl₃) ppm=7.73 (t, J=7.5 Hz, 4H), 7.58-7.48 (m, 4H), 7.43-7.33 (m, 4H), 7.31-7.25 (m, 4H), 6.41 (dd, J=17.6, 11.3 Hz, 1H), 5.67 (t, J=6.9 Hz, 1H), 5.33 (d, J=17.6 Hz, 1H), 5.24 (d, J=11.1 Hz, 1H), 4.57 (t, J=7.8 Hz, 2H), 4.30-4.22 (m, 6H), 4.19-4.12 (m, 2H), 1.54-1.51 (m, 1H). MS (ESI⁺) calc, for C₃₄H₃₂O₅P⁺ [M+H]⁺ 551.2, found 551.4.

Step 2: The intermediate (0.170 g, 0.309 mmol) was reacted according to general procedure XXH, to yield alkyl phosphate XS88 (74 mg, 85%) as the triethylamine salt in a 1:0.9 ratio of phosphate:Et₃N. ¹H NMR (400 MHz, D₂O) ppm=6.59 (dd, J=17.8, 11.3 Hz, 1H), 5.73 (t, J=6.8 Hz, 1H), 5.34 (d, J=17.6 Hz, 1H), 5.25 (d, J=11.4 Hz, 1H), 4.52 (t, J=7.4 Hz, 2H), 4.22 (s, 2H). MS (ESI⁻) calc, for C₆H₁₀O₅P⁻ [M−H]⁻ 193.0, found 193.1.

Triethylamine (E)-3-(hydroxymethyl) pent-2-en-1-yl phosphate (XS89) Step 1: TBDPS-ether XS82 (0.191 g, 0.241 mmol) was reacted according to general procedure XXG. The crude residue was purified by flash chromatography (silica gel, 0-100% EtOAc in heptane) to yield the intermediate allylic alcohol (0.105 g, 79%) as a colorless oil. ¹H NMR (400 MHz, CDCl₃) ppm=7.73 (t, J=7.5 Hz, 4H), 7.59-7.48 (m, 4H), 7.43-7.33 (m, 4H), 7.31-7.22 (m, 4H), 5.49 (t, J=7.0 Hz, 1H), 4.47 (t, J=7.7 Hz, 2H), 4.26 (t, J=6.3 Hz, 4H), 4.18-4.12 (m, 2H), 4.01 (s, 2H), 2.02 (q, J=7.5 Hz, 2H), 0.94 (t, J=7.6 Hz, 3H). MS (ESI⁺) calc, for C₃₄H₃₄₀₅P⁺ [M+H]⁺ 553.5, found 553.2.

Step 2: The intermediate (0.102 g, 0.185 mmol) was reacted according to general procedure XXH, to yield alkyl phosphate XS89 (51 mg, 97%) as the triethylamine salt in a 1:0.7 ratio of phosphate:Et₃N. ¹H NMR (400 MHz, D₂O) ppm=5.51 (t, J=7.0 Hz, 1H), 4.40 (t, J=7.1 Hz, 2H), 4.00 (s, 2H), 2.06 (q, J=7.6 Hz, 2H), 0.92 (t, J=7.6 Hz, 3H). MS (ESI⁻) calc, for C₆H₁₂₀₅P⁻ [M−H]⁻ 195.0, found 195.1.

### Triethylamine (E)-3-(hydroxymethyl) hex-2-en-1-yl phosphate (XS90)

Step 1: TBDPS-ether XS83 (0.345 g, 0.429 mmol) was reacted according to general procedure XXG. The crude residue was purified by flash chromatography (silica gel, 0-100% EtOAc in heptane) to yield the intermediate allylic alcohol (0.213 g, 88%) as a colorless oil. ¹H NMR (400 MHz, CDCl₃) ppm=7.73 (t, J=7.5 Hz, 4H), 7.59-7.48 (m, 4H), 7.43-7.32 (m, 4H), 7.31-7.23 (m, 4H), 5.53 (t, J=6.9 Hz, 1H), 4.48 (t, J=7.7 Hz, 2H), 4.29-4.23 (m, 4H), 4.19-4.13 (m, 2H), 3.99 (s, 2H), 2.02-1.96 (m, 2H), 1.60-1.54 (m, 1H), 1.40-1.31 (m, 2H), 0.85 (t, J=7.3 Hz, 3H). MS (ESI⁺) calc, for C₃₅H₃₅NaO₅P⁺ [M+Na]⁺ 589.2, found 589.5.

Step 2: The intermediate (0.211 g, 0.372 mmol) was reacted according to general procedure XXH, to yield alkyl phosphate XS90 (61 mg, 55%) as the triethylamine salt in a 1:0.7 ratio of phosphate:Et₃N. ¹H NMR (400 MHz, D₂O) ppm=5.56 (t, J=6.9 Hz, 1H), 4.40 (t, J=7.1 Hz, 2H), 3.98 (s, 2H), 2.03 (t, J=7.7 Hz, 2H), 1.34 (sextet, J=7.5 Hz, 2H), 0.80 (t, J=7.4 Hz, 3H). MS (ESI⁻) calc, for C₇H₁₄O₅P⁻ [M−H]⁻ 209.1, found 209.1.

### Triethylamine (E)-3-cyclobutyl-4-hydroxybut-2-en-1-yl phosphate (XS91)

Step 1: TBDPS-ether XS84 (0.515 g, 0.630 mmol) was reacted according to general procedure XXG. The crude intermediate was purified by flash chromatography (silica gel, 0-100% EtOAc in heptane) to yield the intermediate allylic alcohol (0.281 g, 77%) as a colorless oil. ¹H NMR (400 MHz, CDCl₃) ppm=7.73 (t, J=7.7 Hz, 4H), 7.58-7.49 (m, 4H), 7.38 (dt, J=14.6, 7.4 Hz, 4H), 7.31-7.26 (m, 4H), 5.43 (td, J=6.9, 1.4 Hz, 1H), 4.45 (t, J=7.6 Hz, 2H), 4.30-4.21 (m, 4H), 4.18-4.10 (m, 2H), 4.10-4.07 (m, 2H), 3.20 (quint, J=9.1 Hz, 1H), 2.06-1.94 (m, 4H), 1.94-1.81 (m, 1H), 1.77-1.66 (m, 1H). MS (ESI⁺) calc. for C₃₆H₃₆₀₅P⁺ [M+H]⁺ 579.2, found 579.5.

Step 2: The intermediate (0.275 g, 0.475 mmol) was reacted according to general procedure XXH, to yield alkyl phosphate XS91 (0.113 g, 77%) as the triethylamine salt in a 1:0.9 ratio of phosphate:Et₃N. ¹H NMR (400 MHz, D₂O) ppm=5.45 (td, J=6.9, 1.1 Hz, 1H), 4.37 (t, J=7.1 Hz, 2H), 4.07 (s, 2H), 3.34-3.24 (m, 1H), 3.12 (q, J=7.3 Hz, 6H), 2.07-1.90 (m, 5H), 1.89-1.56 (m, 3H), 1.20 (t, J=7.3 Hz, 8H). MS (ESI⁻) calc, for C₈H₁₄O₅P⁻ [M−H]⁻ 221.1, found 221.2.

### Triethylamine (E)-5,5,5-trifluoro-3-(hydroxymethyl) pent-2-en-1-yl phosphate (XS92)

Step 1: TBDPS-ether XS85 (0.117 g, 0.209 mmol) was reacted according to general procedure XXG. The crude intermediate was suspended in heptane and filtered, to yield the intermediate allylic alcohol (0.103 g, 75%) as a white solid. ¹H NMR (400 MHz, CDCl₃) ppm=7.73 (t, J=7.6 Hz, 4H), 7.57-7.46 (m, 4H), 7.38 (dt, J=14.6, 7.3 Hz, 4H), 7.32-7.26 (m, 4H), 5.80 (t, J=6.8 Hz, 1H), 4.44-4.34 (m, 2H), 4.27 (t, J=6.4 Hz, 4H), 4.17-4.10 (m, 2H), 4.05 (s, 2H), 2.83 (q, J=10.9 Hz, 2H). MS (ESI⁺) calc, for C₃₄H₃₁F₃O₅P⁺ [M+H]⁺607.2, found 607.4.

Step 2: The intermediate (0.119 g, 0.196 mmol) was reacted according to general procedure XXH, to yield alkyl phosphate XS92 (27 mg, 41%) as the triethylamine salt in a 1:0.6 ratio of phosphate:Et₃N. ¹H NMR (400 MHz, D₂O) ppm=5.92 (t, J=6.6 Hz, 1H), 4.43 (t, J=7.3 Hz, 2H), 4.06 (s, 2H), 3.12 (q, J=7.4 Hz, 4H), 3.02 (q, J=11.4 Hz, 2H), 1.20 (t, J=7.3 Hz, 6H). MS (ESI⁻) calc, for C₆H₉F₃O₅P⁻ [M−H]⁻ 249.0, found 249.0.

### Triethylamine (E)-3-(hydroxymethyl) pent-2-en-4-yn-1-yl phosphate (XS93)

Step 1: TBDPS-ether XS86 (0.196 g, 0.249 mmol) was reacted according to general procedure XXG. The crude intermediate was purified by flash chromatography (silica gel, 0-100% EtOAc in heptane) to yield the intermediate allylic alcohol (0.103 g, 75%) as a white solid. ¹H NMR (400 MHz, CDCl₃) ppm=7.73 (t, J=7.8 Hz, 4H), 7.59-7.48 (m, 4H), 7.38 (dt, J=14.7, 7.5 Hz, 4H), 7.31-7.23 (m, 4H), 6.04 (t, J=6.6 Hz, 1H), 4.73-4.66 (m, 2H), 4.31-4.23 (m, 4H), 4.19-4.13 (m, 2H), 4.11-4.06 (m, 2H), 3.19 (s, 1H), 1.64 (t, J=6.6 Hz, 1H). MS (ESI⁺) calc, for C₃₄H₃₀₀₅P⁺ [M+H]⁺ 549.2, found 549.4.

Step 2: The intermediate (0.100 g, 0.182 mmol) was reacted according to general procedure XXH, to yield alkyl phosphate XS93 (43 mg, 84%) as the triethylamine salt in a 1:0.9 ratio of phosphate:Et₃N. ¹H NMR (400 MHz, D₂O) ppm=6.15 (t, J=6.4 Hz, 1H), 4.56 (dd, J=8.0, 7.0 Hz, 2H), 4.05 (d, J=0.6 Hz, 2H), 3.60 (s, 1H). MS (ESI⁻) calc, for C₆H₈₀₅P-[M−H]⁻ 191.0, found 191.0.

### 3D. Preparation of Linker-Drugs (XS100-XS105)

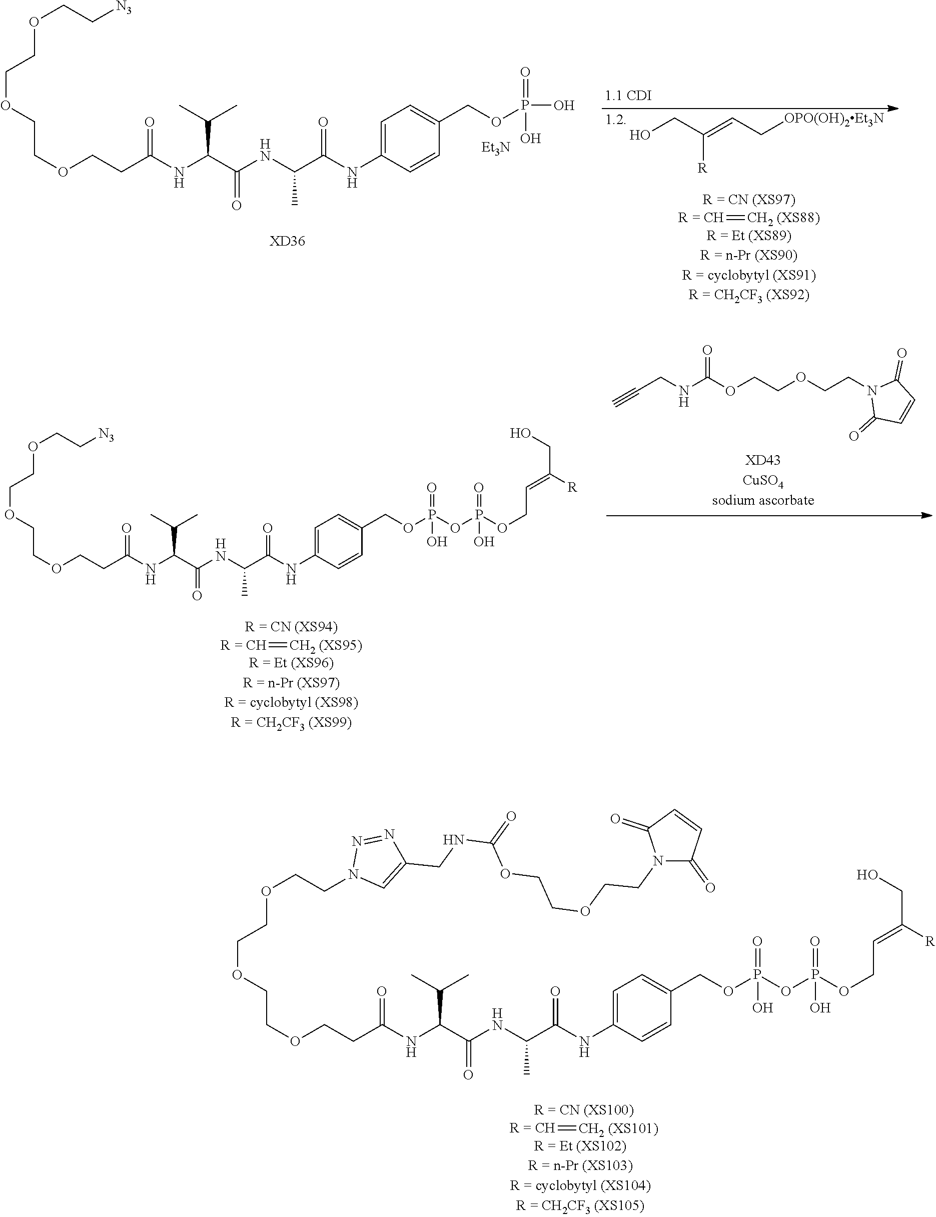

### Pyrophosphate XS94

Alkyl phosphate XD36 (79 mg, 0.11 mmol) was reacted with phosphate XS87 according to general procedure XXD. The crude product was purified by preparative RP-HPLC (25 mM NH₄HCO₃ in MilliQ/MeCN, gradient 90:10 to 50:50), to give after lyophilization, pyrophosphate XS94 (41.0 mg, 45%) as a white solid. ¹H NMR (400 MHz, D₂O) ppm=7.42 (s, 4H), 6.60-6.52 (m, 1H), 4.93 (d, J=6.9 Hz, 2H), 4.64-4.57 (m, 2H), 4.43-4.35 (m, 1H), 4.14-4.11 (m, 1H), 4.11-4.10 (m, 2H), 3.73 (t, J=6.0 Hz, 2H), 3.66-3.57 (m, 10H), 3.44-3.40 (m, 2H), 2.62-2.48 (m, 2H), 2.11-1.98 (m, 1H), 1.43 (d, J=7.3 Hz, 3H), 0.92 (d, J=6.6 Hz, 3H), 0.90 (d, J=6.6 Hz, 3H). MS (ESI⁻) calcd, for C₂₉H₄₄N₇O₁₄P2 [M−H]⁻ 776.2, found 776.6.

### Pyrophosphate XS95

Alkyl phosphate XD36 (79 mg, 0.11 mmol) was reacted with phosphate XS88 according to general procedure XXD. The crude product was purified by preparative RP-HPLC (25 mM NH₄HCO₃ in MilliQ/MeCN, gradient 90:10 to 50:50), to give after lyophilization, pyrophosphate XS95 (50.1 mg, 55%) as a white solid. ¹H NMR (400 MHz, D₂O) ppm=7.42 (s, 4H), 6.48 (dd, J=17.6, 11.3 Hz, 1H), 5.69 (t, J=6.8 Hz, 1H), 5.30 (d, J=17.6 Hz, 1H), 5.20 (d, J=11.4 Hz, 1H), 4.93 (d, J=6.5 Hz, 2H), 4.57-4.50 (m, 2H), 4.40 (q, J=7.2 Hz, 1H), 4.17 (s, 2H), 4.12 (d, J=7.1 Hz, 1H), 3.73 (t, J=6.0 Hz, 2H), 3.66-3.57 (m, 10H), 3.45-3.40 (m, 2H), 2.63-2.48 (m, 2H), 2.12-1.98 (m, 1H), 1.43 (d, J=7.1 Hz, 3H), 0.92 (d, J=6.6 Hz, 3H), 0.91 (d, J=6.6 Hz, 3H). MS (ESI⁻) calcd, for C₃₀H₄₇N₆O₁₄P2 [M−H]⁻ 777.3, found 777.4.

### Pyrophosphate XS96

Alkyl phosphate XD36 (100 mg, 0.142 mmol) was reacted with phosphate XS89 according to general procedure XXD. The crude product was purified by preparative RP-HPLC (25 mM NH₄HCO₃ in MilliQ/MeCN, gradient 90:10 to 50:50), to give after lyophilization, pyrophosphate XS96 (48.6 mg, 42%) as a white solid. ¹H NMR (400 MHz, D₂O) ppm=7.44 (s, 4H), 5.49 (t, J=6.8 Hz, 1H), 4.99-4.92 (m, 2H), 4.48-4.36 (m, 3H), 4.13 (d, J=7.1 Hz, 1H), 3.97 (s, 2H), 3.74 (t, J=5.9 Hz, 2H), 3.69-3.57 (m, 10H), 3.48-3.40 (m, 2H), 2.64-2.49 (m, 2H), 2.12-2.03 (m, 1H), 2.00 (q, J=7.6 Hz, 2H), 1.44 (d, J=7.1 Hz, 3H), 0.95-0.85 (m, 9H). MS (ESI⁻) calcd, for C₃₀H₄₉N₆O₁₄P₂⁻ [M−H]⁻ 779.3, found 779.5.

### Pyrophosphate XS97

Alkyl phosphate XD36 (54 mg, 0.077 mmol) was reacted with phosphate XS90 according to general procedure XXD. The crude product was purified by preparative RP-HPLC (25 mM NH₄HCO₃ in MilliQ/MeCN, gradient 90:10 to 50:50), to give after lyophilization, pyrophosphate XS97 (37.3 mg, 59%) as a white solid. ¹H NMR (400 MHz, D₂O) ppm=7.42 (s, 4H), 5.52 (t, J=6.8 Hz, 1H), 4.93 (d, J=6.4 Hz, 2H), 4.45-4.37 (m, 3H), 4.12 (d, J=7.0 Hz, 1H), 3.94 (s, 2H), 3.73 (t, J=5.9 Hz, 2H), 3.65-3.56 (m, 10H), 3.45-3.40 (m, 2H), 2.63-2.48 (m, 2H), 2.10-2.00 (m, 1H), 1.95 (t, J=7.6 Hz, 2H), 1.43 (d, J=7.3 Hz, 3H), 1.28 (sxt, J=7.5 Hz, 2H), 0.96-0.86 (m, 6H), 0.75 (t, J=7.3 Hz, 3H). MS (ESI⁻) calcd, for C₃₁H₅₁N₆O₁₄P₂⁻ [M−H]⁻ 793.3, found 793.5.

### Pyrophosphate XS98

Alkyl phosphate XD36 (105 mg, 0.149 mmol) was reacted with phosphate XS91 according to general procedure XXD. The crude product was purified by preparative RP-HPLC (25 mM NH₄HCO₃ in MilliQ/MeCN, gradient 90:10 to 50:50), to give after lyophilization, pyrophosphate XS98 (64.1 mg, 51%) as a white solid. ¹H NMR (400 MHz, D₂O) ppm=7.43 (s, 4H), 5.43-5.37 (m, 1H), 4.94 (d, J=6.6 Hz, 2H), 4.43-4.34 (m, 3H), 4.11 (d, J=7.1 Hz, 1H), 4.01 (s, 2H), 3.73 (t, J=6.0 Hz, 2H), 3.64-3.57 (m, 10H), 3.45-3.40 (m, 2H), 3.14 (quint, J=9.0 Hz, 1H), 2.62-2.47 (m, 2H), 2.10-1.99 (m, 1H), 1.98-1.82 (m, 4H), 1.82-1.71 (m, 1H), 1.65-1.55 (m, 1H), 1.42 (d, J=7.1 Hz, 3H), 0.91 (d, J=6.5 Hz, 3H), 0.90 (d, J=6.5 Hz, 3H). MS (ESI⁻) calcd, for C₃₂H₅₁N₆O₁₄P₂⁻ [M−H]⁻ 805.3, found 805.5.

### Pyrophosphate XS99

Alkyl phosphate XD36 (43 mg, 0.061 mmol) was reacted with phosphate XS92 according to general procedure XXD. The crude product was purified by preparative RP-HPLC (25 mM NH₄HCO₃ in MilliQ/MeCN, gradient 90:10 to 50:50), to give after lyophilization, pyrophosphate XS99 (12.3 mg, 23%) as a white solid. ¹H NMR (400 MHz, D₂O) ppm=7.40 (s, 4H), 5.85 (t, J=6.3 Hz, 1H), 4.90 (br s, 2H), 4.47-4.40 (m, 2H), 4.40-4.34 (m, 1H), 4.14-4.07 (m, 1H), 3.98 (s, 2H), 3.71 (s, 2H), 3.64-3.53 (m, 10H), 3.43-3.37 (m, 2H), 2.91 (q, J=11.4 Hz, 2H), 2.61-2.45 (m, 2H), 2.03 (dq, J=13.7, 6.9 Hz, 1H), 1.40 (d, J=7.3 Hz, 3H), 0.89 (d, J=6.5 Hz, 3H), 0.88 (d, J=6.6 Hz, 3H). MS (ESI⁻) calcd. for C₃₀H₄₆F₃N₆O₁₄P₂⁻ [M−H]⁻ 833.3, found 833.6.

### Linker-Drug XS100

Azide XS94 (36 mg, 0.044 mmol) was reacted with alkyne XD43 according to general procedure XXE. Purification by preparative RP-HPLC (25 mM NH₄HCO₃ in MilliQ/MeCN, gradient 90:10 to 50:50), afforded after lyophilization pyrophosphate XS100 (15.5 mg, 33%) as a white solid. ¹H NMR (400 MHz, D₂O) ppm=7.90 (br s, 1H), 7.41 (s, 4H), 6.74 (s, 2H), 6.56 (t, J=5.9 Hz, 1H), 4.98-4.89 (m, 2H), 4.64-4.58 (m, 2H), 4.53 (t, J=4.8 Hz, 2H), 4.38 (q, J=7.2 Hz, 1H), 4.31 (br s, 2H), 4.16-4.03 (m, 5H), 3.88 (t, J=4.9 Hz, 2H), 3.70 (t, J=5.9 Hz, 2H), 3.66-3.59 (m, 6H), 3.57-3.47 (m, 8H), 2.61-2.47 (m, 2H), 2.11-1.97 (m, 1H), 1.41 (d, J=7.1 Hz, 3H), 0.90 (d, J=7.0 Hz, 3H), 0.88 (d, J=6.9 Hz, 3H). MS (ESI⁻) calcd, for C₄₁H₅₈N₉O₁₉P₂⁻ [M−H]⁻ 1042.3, found 1042.8.

### Linker-Drug XS101

Azide XS95 (44 mg, 0.054 mmol) was reacted with alkyne XD43 according to general procedure XXE. Purification by preparative RP-HPLC (25 mM NH₄HCO₃ in MilliQ/MeCN, gradient 90:10 to 50:50), afforded after lyophilization pyrophosphate XS101 (19.8 mg, 34%) as a white solid. ¹H NMR (400 MHz, D₂O) ppm=7.92 (br s, 1H), 7.41 (s, 4H), 6.74 (s, 2H), 6.48 (dd, J=17.8, 11.3 Hz, 1H), 5.73-5.65 (m, 1H), 5.30 (d, J=17.6 Hz, 1H), 5.20 (d, J=11.1 Hz, 1H), 4.92 (td, J=1.9, 1.0 Hz, 2H), 4.59-4.50 (m, 4H), 4.38 (q, J=7.3 Hz, 1H), 4.31 (tdd, J=4.2, 2.3, 1.3 Hz, 2H), 4.17 (s, 2H), 4.14-4.04 (m, 3H), 3.88 (t, J=4.8 Hz, 2H), 3.71 (t, J=5.9 Hz, 2H), 3.66-3.58 (m, 6H), 3.58-3.48 (m, 8H), 2.62-2.47 (m, 2H), 2.10-1.99 (m, 1H), 1.42 (d, J=7.1 Hz, 3H), 0.91 (d, J=7.0 Hz, 3H), 0.88 (d, J=6.9 Hz, 3H). MS (ESI⁻) calcd, for C₄₂H₆₁N₈O₁₉P₂⁻ [M−H]⁻ 1043.4, found 1043.6.

### Linker-Drug XS102

Azide XS96 (42 mg, 0.052 mmol) was reacted with alkyne XD43 according to general procedure XXE. Purification by preparative RP-HPLC (25 mM NH₄HCO₃ in MilliQ/MeCN, gradient 90:10 to 50:50), afforded after lyophilization pyrophosphate XS102 (8.9 mg, 16%) as a white solid. ¹H NMR (400 MHz, D₂O) ppm=7.99-7.87 (m, J=1.0 Hz, 1H), 7.41 (s, 4H), 6.74 (s, 2H), 5.47 (br s, 1H), 4.93 (br s, 2H), 4.53 (br t, J=4.6 Hz, 2H), 4.46-4.40 (m, 2H), 4.40-4.34 (m, 1H), 4.31 (d, J=1.0 Hz, 2H), 4.15-4.04 (m, 3H), 3.95 (s, 2H), 3.87 (t, J=4.6 Hz, 2H), 3.70 (t, J=5.9 Hz, 2H), 3.62 (s, 6H), 3.58-3.46 (m, 8H), 2.62-2.46 (m, 2H), 2.10-1.93 (m, 3H), 1.41 (d, J=7.1 Hz, 3H), 0.94-0.81 (m, 9H). MS (ESI⁻) calcd. for C₄₂H₆₃N₈O₁₉P₂⁻ [M−H]⁻ 1045.4, found 1045.6.

### Linker-Drug XS103

Azide XS97 (32 mg, 0.038 mmol) was reacted with alkyne XD43 according to general procedure XXE. Purification by preparative RP-HPLC (25 mM NH₄HCO₃ in MilliQ/MeCN, gradient 90:10 to 50:50), afforded after lyophilization pyrophosphate XS103 (9.0 mg, 22%) as a white solid. ¹H NMR (400 MHz, D₂O) ppm=7.89 (br s, 1H), 7.41 (s, 4H), 6.74 (s, 2H), 5.51 (t, J=6.3 Hz, 1H), 4.92 (br s, 2H), 4.53 (br s, 2H), 4.46-4.35 (m, 3H), 4.31 (br s, 2H), 4.16-4.04 (m, 3H), 3.93 (s, 2H), 3.87 (t, J=4.9 Hz, 2H), 3.70 (t, J=5.9 Hz, 2H), 3.66-3.58 (m, 6H), 3.57-3.47 (m, 8H), 2.61-2.47 (m, 2H), 2.10-1.99 (m, 1H), 1.94 (t, J=7.6 Hz, 2H), 1.41 (d, J=7.3 Hz, 3H), 1.27 (sxt, J=7.4 Hz, 2H), 0.90 (d, J=7.3 Hz, 3H), 0.88 (d, J=7.0 Hz, 3H), 0.74 (t, J=7.3 Hz, 3H). MS (ESI⁻) calcd, for C₄₃H₆₅N₈O₁₉P₂⁻ [M−H]⁻ 1059.4, found 1059.8.

### Linker-Drug XS104

Azide XS98 (56 mg, 0.067 mmol) was reacted with alkyne XD43 according to general procedure XXE. Purification by preparative RP-HPLC (25 mM NH₄HCO₃ in MilliQ/MeCN, gradient 90:10 to 50:50), afforded after lyophilization pyrophosphate XS104 (22.7 mg, 31%) as a white solid. ¹H NMR (400 MHz, D₂O) ppm=7.91 (br s, 1H), 7.42 (s, 4H), 6.74 (s, 2H), 5.41 (t, J=5.4 Hz, 1H), 4.93 (br s, 2H), 4.53 (t, J=4.9 Hz, 2H), 4.44-4.34 (m, 3H), 4.31 (br s, 2H), 4.10-4.10 (m, 1H), 4.12 (d, J=7.0 Hz, 1H), 4.10-4.04 (m, 2H), 4.01 (s, 2H), 3.88 (t, J=4.9 Hz, 2H), 3.70 (t, J=5.9 Hz, 2H), 3.66-3.58 (m, 6H), 3.57-3.46 (m, 8H), 3.14 (quint, J=9.0 Hz, 1H), 2.62-2.46 (m, 2H), 2.10-1.99 (m, 1H), 1.98-1.82 (m, 4H), 1.82-1.71 (m, 1H), 1.65-1.54 (m, 1H), 1.41 (d, J=7.1 Hz, 3H), 0.90 (d, J=6.9 Hz, 3H), 0.88 (d, J=6.9 Hz, 3H). MS (ESI) calcd, for C₄₄H₆₅N₈O₁₉P₂⁻ [M−H]⁻ 1071.4, found 1071.7.

### Linker-Drug XS105

Azide XS99 (11 mg, 0.012 mmol) was reacted with alkyne XD43 according to general procedure XXE. Purification by preparative RP-HPLC (25 mM NH₄HCO₃ in MilliQ/MeCN, gradient 90:10 to 50:50), afforded after lyophilization pyrophosphate XS105 (3.3 mg, 24%) as a white solid. ¹H NMR (400 MHz, D₂O) ppm=7.90 (br s, 1H), 7.41 (s, 4H), 6.74 (s, 2H), 5.87 (t, J=5.5 Hz, 1H), 4.92 (br s, 2H), 4.53 (t, J=4.8 Hz, 2H), 4.45 (br s, 2H), 4.42-4.34 (m, 1H), 4.31 (br s, 2H), 4.15-4.05 (m, 3H), 4.00 (s, 2H), 3.87 (t, J=4.9 Hz, 2H), 3.70 (t, J=5.9 Hz, 2H), 3.66-3.58 (m, 6H), 3.57-3.48 (m, 8H), 2.93 (q, J=11.3 Hz, 2H), 2.61-2.46 (m, 2H), 2.11-1.97 (m, 1H), 1.41 (d, J=7.3 Hz, 3H), 0.90 (d, J=7.1 Hz, 3H), 0.88 (d, J=7.0 Hz, 3H). MS (ESI⁻) calcd, for C₄₂H₆₀F₃N₈O₁₉P₂⁻ [M−H]⁻ 1099.3, found 1099.7.

### Preparation of Linker-Drug (XS107)

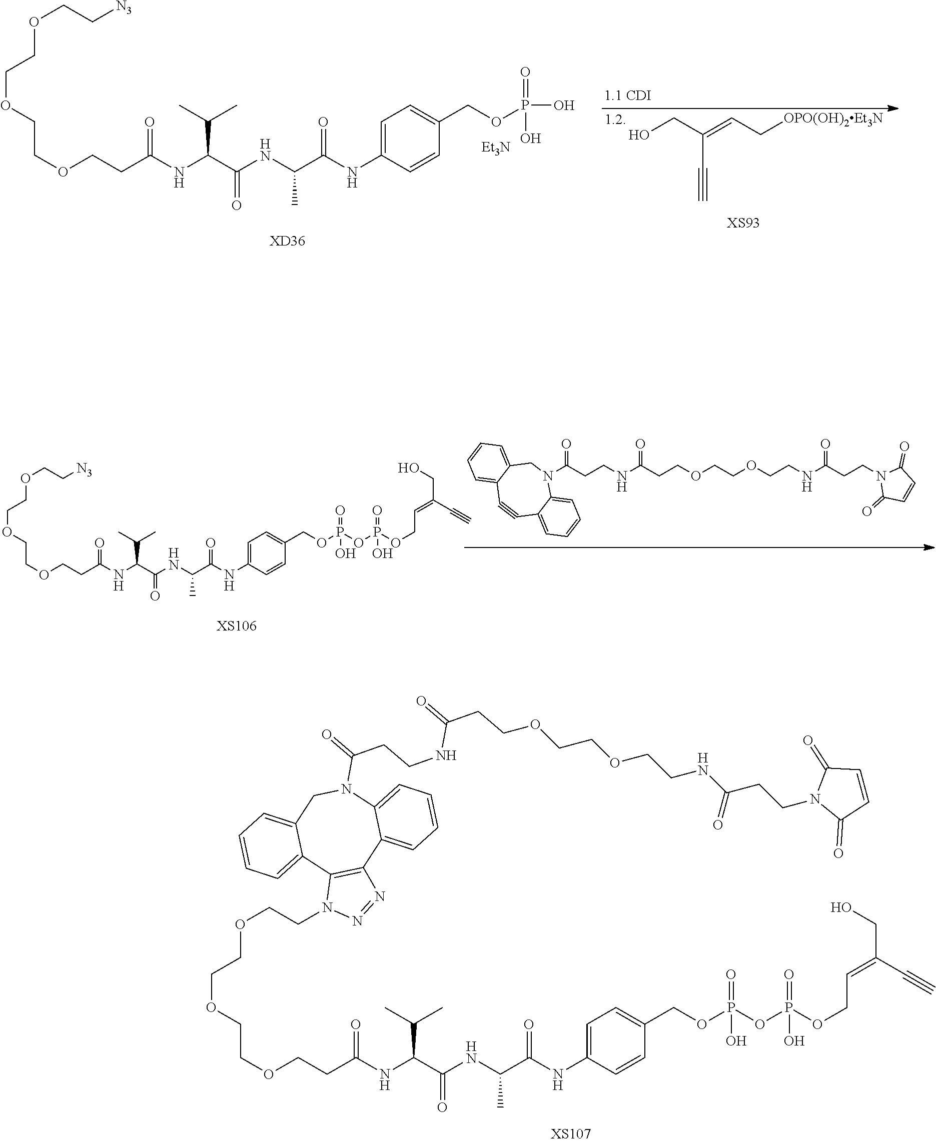

### Pyrophosphate XS106

Alkyl phosphate XD36 (90 mg, 0.128 mmol) was reacted with phosphate XS93 according to general procedure XXD. The crude product was purified by preparative RP-HPLC (25 mM NH₄HCO₃ in MilliQ/MeCN, gradient 90:10 to 50:50), to give after lyophilization, pyrophosphate XS106 (50.1 mg, 48%) as a white solid. ¹H NMR (400 MHz, D₂O) ppm=7.40 (s, 4H), 6.09 (t, J=6.4 Hz, 1H), 4.93-4.88 (m, 2H), 4.60-4.54 (m, 2H), 4.37 (q, J=7.1 Hz, 1H), 4.09 (d, J=7.0 Hz, 1H), 3.98 (s, 2H), 3.71 (t, J=6.0 Hz, 2H), 3.64-3.54 (m, 11H), 3.43-3.37 (m, 2H), 2.61-2.45 (m, 2H), 2.10-1.96 (m, 1H), 1.41 (d, J=7.3 Hz, 3H), 0.89 (d, J=6.6 Hz, 3H), 0.88 (d, J=6.6 Hz, 3H). MS (ESI⁻) calcd, for C₃₀H₄₅N₆O₁₄P₂⁻ [M−H]⁻ 775.2, found 775.4.

### Linker-Drug XS107

Azide XS106 (18 mg, 0.022 mmol) was dissolved in water (120 μL) and a solution of DBCO-PEG2-Maleimide (18 mg, 0.030 mmol) was added. The reaction mixture was stirred at RT for 45 min and subsequently purified by preparative RP-HPLC (25 mM NH₄HCO₃ in MilliQ/MeCN, gradient 90:10 to 50:50), to afford after lyophilization pyrophosphate XS107 (14.1 mg, 46%) as a white solid. MS (ESI⁻) calcd, for C₆₂H₇₉N₁₀O₂₁P₂⁻ [M−H]⁻ 1361.5, found 1361.9.

### Example 4: Synthesis of Conjugates of Rituximab with Linker Drug Compounds XD73, XS54-XS58, XS100-XS105 and XS107 with DAR 2

To a solution of antibody (10-12 mg/mL) was added TRIS (1 vol %, 1 M, pH 8), EDTA (4 vol %, 25 mM) and TCEP (5 mM in water). The resulting solution was incubated at RT for 2 h. After incubation, the reduced antibody was rebuffered to 4.2 mM histidine, 50 mM trehalose pH 6 and treated with dimethylacetamide (DMA) and linker-drug compound (LD) (10 mM in DMA, >1.5 eq/SH). Final DMA content was ~10 vol %. The resulting mixture was roller mixed in the dark at RT overnight. Activated carbon was added and the suspension was roller mixed in the dark for 1 h, filtered, washed with 4.2 mM histidine, 50 mM trehalose pH 6. The solution was rebuffered to 4.2 mM histidine, 50 mM trehalose pH 6 and sterile filtered.

In order to approximate the DAR (pAg to antibody ration) of the conjugates with a target DAR of 2, surrogate conjugation was performed with the hydrophobic seco-DUBA payload (SYD980, described in a.o. WO2015/177360), that allows facile DAR determination via HIC. The resulting average DAR for conjugates where the target DAR is 2, is reflected in table 1.

TABLE 1
Synthesized Conjugates with antibody and Linker-drug (LD) compound used in
synthesis

| | | | | Avg. | |
|---|---|---|---|---|---|
| LD | LD Structure | Conjugate | MoAb | DAR | % HMW |
| XD73 | 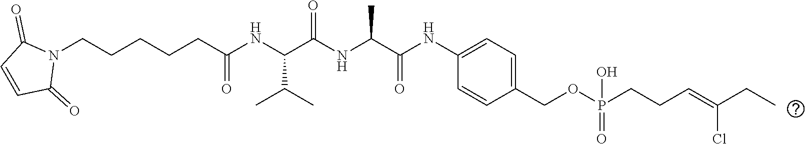 [SMILES: CC/C(Cl)=C/CCP(=O)(O)OCc1ccc(NC(=O)[C@H](C)NC(=O)[C@@H](NC(=O)CCCCCN2C(=O)C=CC2=O)C(C)C)cc1] | ADC- XD37-r ADC- XD37i | rituxi mab (anti- CD20) Isotype (Anti-HIV) | 2.0 1.9 | 0.7 0.9 |
| XS58 | 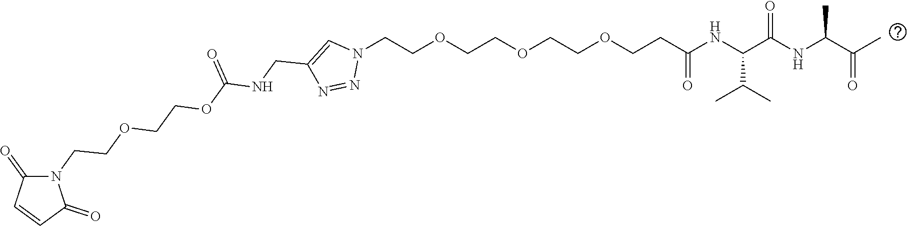 [SMILES: CC(=O)[C@H](C)NC(=O)[C@@H](NC(=O)CCOCCOCCOCCn1cc(CNC(=O)OCCOCCN2C(=O)C=CC2=O)nn1)C(C)C] | ADC- XS58-r ADC- XS58-i | rituxi mab isotype | 2.0 1.9 | 0.7 1.3 |
| XS56 | 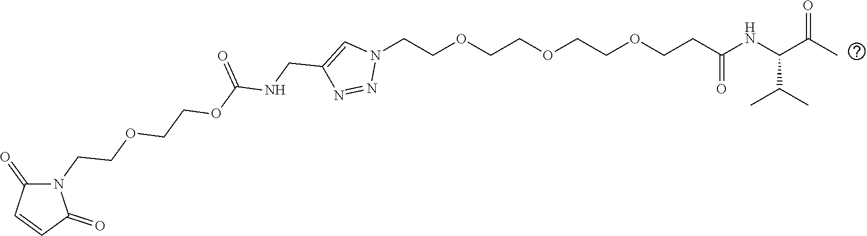 [SMILES: CC(=O)[C@@H](NC(=O)CCOCCOCCOCCn1cc(CNC(=O)OCCOCCN2C(=O)C=CC2=O)nn1)C(C)C] | ADC- XS56-r ADC- XS56-i | rituxi mab isotype | 2.0 1.9 | 0.7 1.3 |
| XS54 | 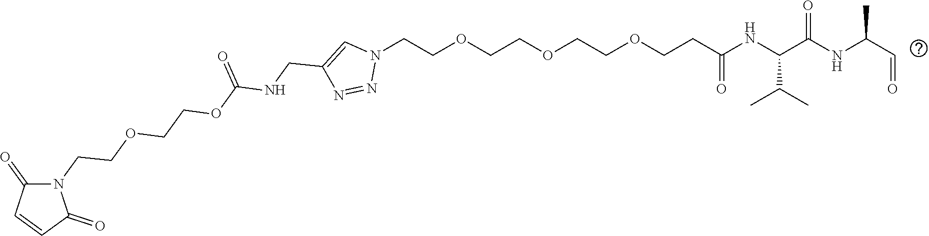 [SMILES: CC(C)[C@H](NC(=O)CCOCCOCCOCCn1cc(CNC(=O)OCCOCCN2C(=O)C=CC2=O)nn1)C(=O)N[C@@H](C)C=O] | ADC- XS54-r ADC- XS54-i | rituxi mab isotype | 2.0 1.9 | 0.7 1.3 |
| XS57 | 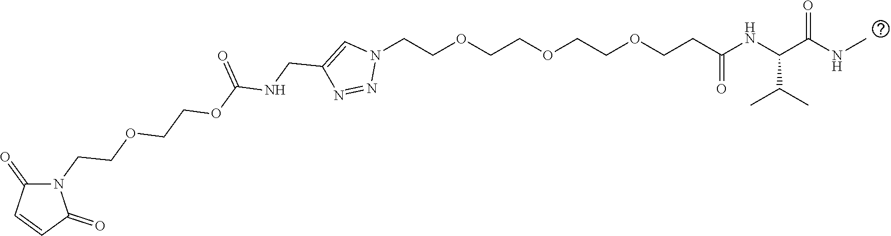 [SMILES: CNC(=O)[C@@H](NC(=O)CCOCCOCCOCCn1cc(CNC(=O)OCCOCCN2C(=O)C=CC2=O)nn1)C(C)C] | ADC- XS57-r ADC- XS57-i | rituxi mab isotype | 2.0 1.9 | 0.7 1.2 |
| XS55 | 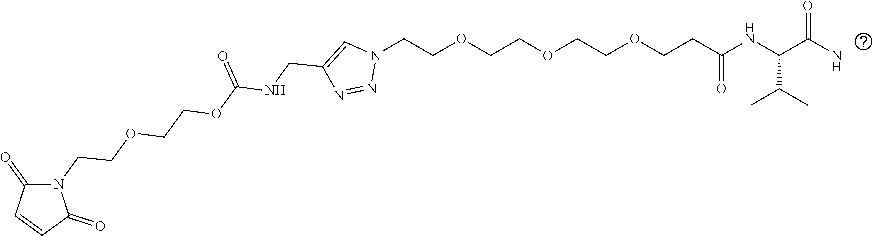 [SMILES: CC(C)[C@H](NC(=O)CCOCCOCCOCCn1cc(CNC(=O)OCCOCCN2C(=O)C=CC2=O)nn1)C(N)=O] | ADC- XS55-r ADC- XS55-i | rituxi mab isotype | 2.4 2.1 | 0.6 1.1 |
| XS 100 | 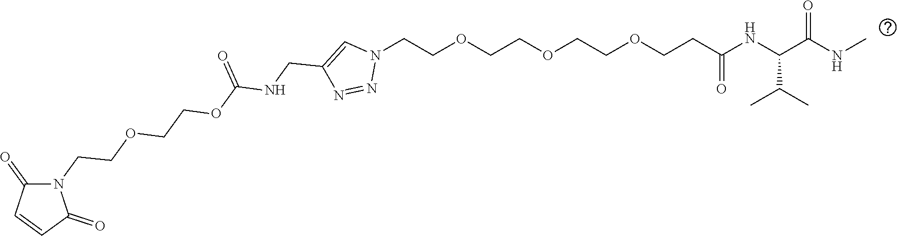 [SMILES: CNC(=O)[C@@H](NC(=O)CCOCCOCCOCCn1cc(CNC(=O)OCCOCCN2C(=O)C=CC2=O)nn1)C(C)C] | ADC- XS100-r ADC- XS100-i | rituxi mab isotype | 2.4 2.1 | 0.6 1.2 |
| XS 102 | 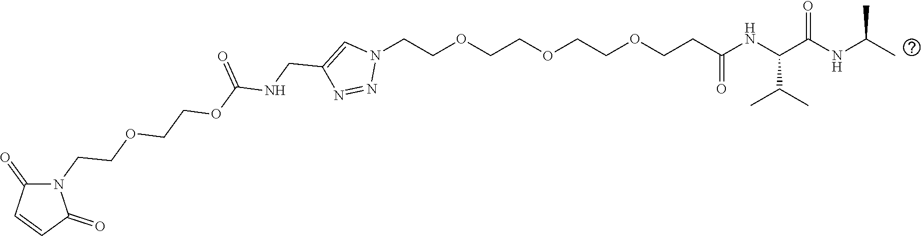 | ADC- XS102-r ADC- XS102-i | rituxi mab isotype | 2.4 2.1 | 0.6 1.3 |
| XS 101 | 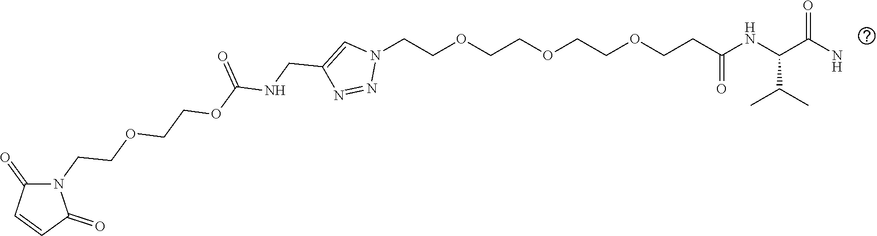 [SMILES: CC(C)[C@H](NC(=O)CCOCCOCCOCCn1cc(CNC(=O)OCCOCCN2C(=O)C=CC2=O)nn1)C(N)=O] | ADC- XS101-r ADC- XS101-i | rituxi mab isotype | 2.4 2.1 | 0.7 1.5 |
| XS 103 | 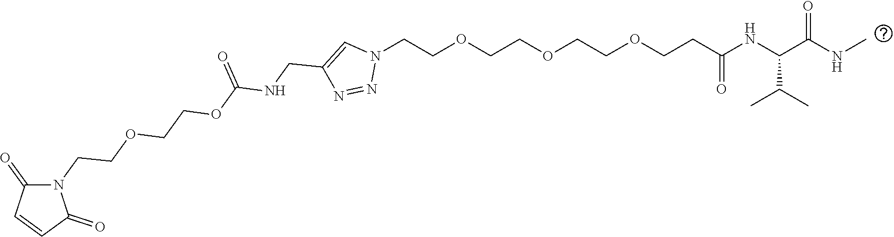 [SMILES: CNC(=O)[C@@H](NC(=O)CCOCCOCCOCCn1cc(CNC(=O)OCCOCCN2C(=O)C=CC2=O)nn1)C(C)C] | ADC- XS103-r ADC- XS103-i | rituxi mab isotype | 2.4 1.7 | 0.8 1.3 |
| XS 105 | 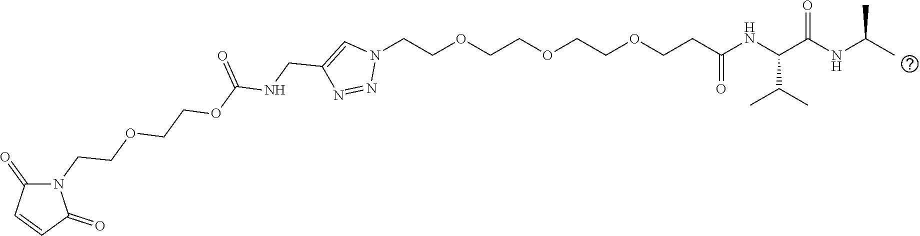 | ADC- XS105-r ADC- XS105-i | rituxi mab isotype | 2.4 1.7 | 0.6 1.4 |
| XS 104 | 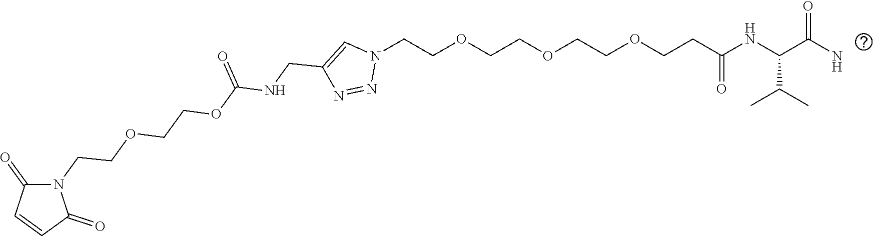 [SMILES: CC(C)[C@H](NC(=O)CCOCCOCCOCCn1cc(CNC(=O)OCCOCCN2C(=O)C=CC2=O)nn1)C(N)=O] | ADC- XS104-r ADC- XS104-i | rituxi mab isotype | 2.4 1.7 | 0.7 1.2 |
| XS 107 | 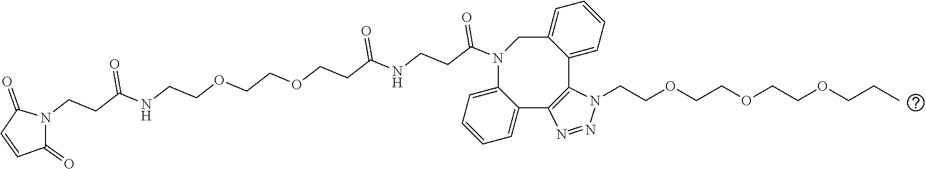 [SMILES: CCCOCCOCCOCCn1nnc2c1-c1ccccc1CN(C(=O)CCNC(=O)CCOCCOCCNC(=O)CCN1C(=O)C=CC1=O)c1ccccc1-2] | ADC- XS107-r ADC- XS107-i | rituxi mab isotype | 2.1 2.9 | 2.5 3.5 |

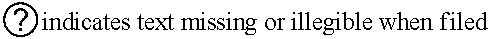

### Example 5: Activity of Phosphoantigen (pAg) Conjugates (ADCs) on Gamma Delta T-Cells

Multiple synthesized linker drug compounds were conjugated to rituximab (anti-CD20) and resulting ADCs were tested for their ability to bind Raji cells and to activate Vdelta2 gammadelta T-cells after overnight incubation with CD20-positive Raji cells. The linker drug compounds that were conjugated to rituximab, and resulting ADCs, are within the list reflected in Table 1. The pretreated Raji cells were cocultured with peripheral blood mononuclear cells (PBMCs) and activation (IFNγ production) and degranulation (CD107a) of Vdelta2 gammadelta T-cells and NK-cells was determined using multicolor flow cytometry.

### Material and Methods

### Cellular Binding

The CD20-positive Burkitt's Lymphoma human tumor cell line Raji (DSMZ, the German collection of Microorganisms and cell cultures GmbH) was used for in vitro experiments. Raji cells were cultured in complete growth medium (CGM): RPMI-1640 (Lonza) supplemented with 10% Heat-inactivated (HI) Fetal Bovine Serum (FBS) (Gibco) and 80 U/mL Penicillin-Streptomycin solution (Lonza). Raji cells were maintained at 37° C. in a humidified incubator containing 5% CO₂ and sub-cultured twice a week. For cellular binding in a 96 well plate, 100,000 Raji cells/well were washed twice with ice-cold FACS buffer (PBS 1×, 0.1% v/w BSA, 0.02% v/v Sodium Azide), followed by the addition of a concentration range of 50 μL/well of an anti-CD20 ADC, naked antibody (e.g. rituximab) or non-binding isotype control ADC diluted in ice-cold FACS buffer. After an incubation time of 30 minutes at 4° C., the cells were washed twice with ice-cold FACS buffer. Then, 50 μL/well APC-conjugated secondary F(ab′)₂ goat anti-Human IgG (Fc fragment specific, Jackson Immuno research, 109-136-098, 1:6000) was added. After 30 minutes at 4° C., cells were washed twice and resuspended in 150 μL ice-cold FACS buffer. Fluorescence intensities were determined by flow cytometry using the NovoCyte (Agilent) and determined as the median fluorescence intensity (MFI). Curves were fitted by nonlinear regression with a variable slope (four parameters) in GraphPad Prism version 9. EC₅₀ values were calculated in GraphPad Prism as the concentration in μg/mL that gives a response halfway between bottom and top of the curve. Binding experiments were performed in three independent experiments.

### Functional Assay (Determining Gammadelta T-Cell Activity Induced by Different pAg Conjugates).

For stimulation with ADCs according to the invention, Raji cells were harvested, diluted to a concentration of 5×10⁶ cells/mL and 50 μL (equivalent to 250,000 cells/well) of this cell suspension was seeded into a 96-well plate. A 2-times concentrated, 5-fold serial dilution of the ADCs or rituximab were prepared in complete growth medium (CGM, RPMI-1640 (Lonza) supplemented with 10% HI FBS (Gibco) and 80 U/mL Penicillin-Streptomycin solution (Lonza). Plated Raji cells were incubated overnight in a humidified incubator with 5% CO₂ at 37° C. with 50 μL/well of the serially-diluted compounds (total 100 μL/well). The following day, the 96 well plate with Raji cells and ADCs or rituximab was washed by adding 100 μL/well CGM, centrifugation at 300×g for 3 minutes at room temperature, and removal of supernatant in order to remove excessive unbound compound. As a source of immune cells, frozen PBMCs of a healthy human donor were thawed, resuspended in CGM and placed overnight in a humidified incubator with 5% CO₂ at 37° C. to let the cells recover. The recovered PBMCs were harvested, counted and diluted to a concentration of 10×106 cells/mL in CGM, and 50 μL/well (equivalent to 0.5×10⁶ cells/well) was added to the Raji cells. A 2-times concentrated anti-CD107a BV421 (BioLegend) solution was prepared in CGM, containing GolgiStop (Monensin) and GolgiPlug (Brefeldin A) (BD Biosciences), and 50 μL/well was added to the Raji-PBMCs co-culture. Samples were incubated for 6 hours in a humidified incubator with 5% CO₂ at 37° C. For staining of immune cell subsets, a multicolor antibody staining cocktail was prepared in Brilliant Stain buffer (BD Biosciences), containing anti-CD3 BV711 (BioLegend), anti-CD56 BV510 (BioLegend), Fixable Viability Stain 780 (BD Biosciences), anti-CD16 FITC (BD Biosciences), FcR Blocking Reagent (Miltenyi Biotec) and anti-TCR Vdelta2 APC (BioLegend). After the 6 hours incubation period, the plate was centrifuged at 300×g for 3 minutes at room temperature and supernatant was discarded. The pellet was re-suspended in 50 μL antibody cocktail and incubated for 30 minutes on ice, protected from light. The plate was washed twice by adding 100 μL ice-cold FACS buffer (PBS 1×, 0.1% v/w BSA, 0.02% v/v Sodium Azide), followed by centrifugation at 300×g for 3 minutes and discarding of the supernatant. Cells were fixed and permeabilized using 100 μL/well Cytofix/Cytoperm Solution (BD Bioscience) and were incubated for 20 minutes on ice, protected from light. Cells were washed three times by adding 150 μL BD Perm/wash solution containing saponin (dilute 10× BD Perm/Wash buffer in distilled H₂O, to make a 1×solution prior to use), followed by centrifugation at 300×g for 3 minutes and discarding of the supernatant. Finally, cells were re-suspended in FACS buffer and stored overnight in the fridge at 4° C., protected from light. On the third day, stained PBMCs/Raji cells were washed once in 150 μL BD Perm/wash solution followed by centrifugation at 300×g for 3 minutes and discarding of the supernatant. The pellet was re-suspended in a mix of 50 μL anti-IFNγ PE-Cy7 (BioLegend) diluted in Perm/Wash solution and incubated for 30 minutes on ice, protected from light. After incubation the plate was washed once with 150 μL ice-cold FACS buffer, followed by centrifugation at 300×g for 3 minutes and discarding of the supernatant. Thereafter, the cell pellet was resuspended in 100 μL FACS buffer and samples were analyzed using the NovoCyte (Agilent). Curves were fitted by nonlinear regression with a variable slope (four parameters) in GraphPad Prism version 9. EC₅₀ values were calculated in GraphPad Prism as the concentration in ug/mL that gives a response halfway between bottom and top of the curve. Each compound was tested in at least two independent experiments with a different donor.

### Results/Conclusion

Multiple rituximab ADCs and non-binding controls were generated with a drug-to-antibody-ratio (DAR) of ~2. Their binding to Raji cells was comparable to naked rituximab (Table 2) and the non-binding isotype controls did not show binding (data not shown).

The generated ADCs were tested for their ability to induce Vdelta2 gammadelta T-cell activation after overnight incubation with Raji cells, followed by a 6 hours coculture with

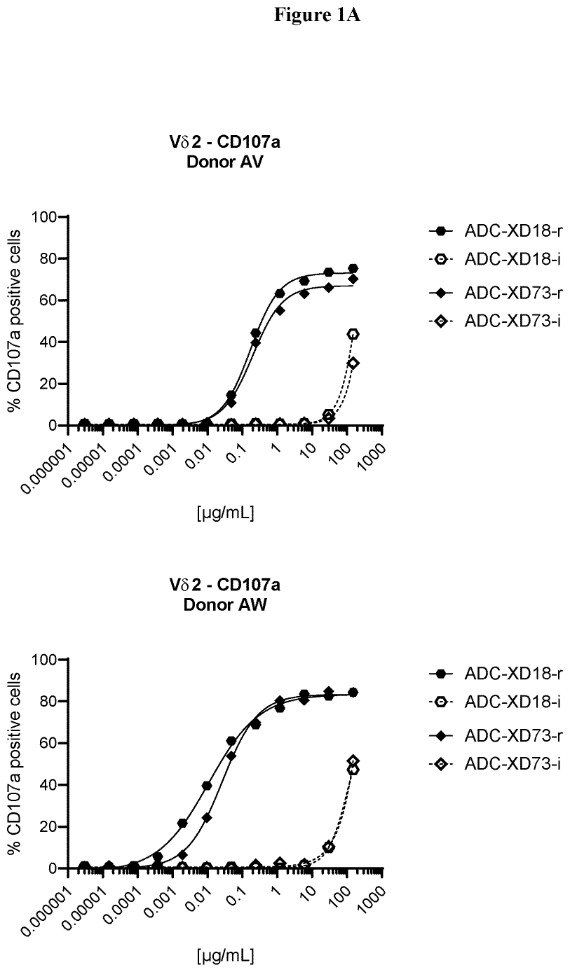

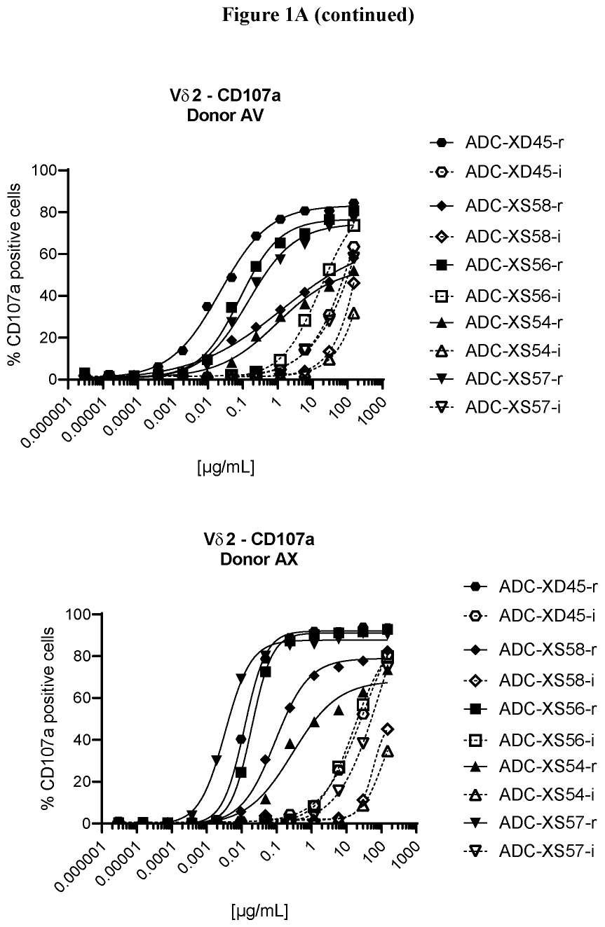

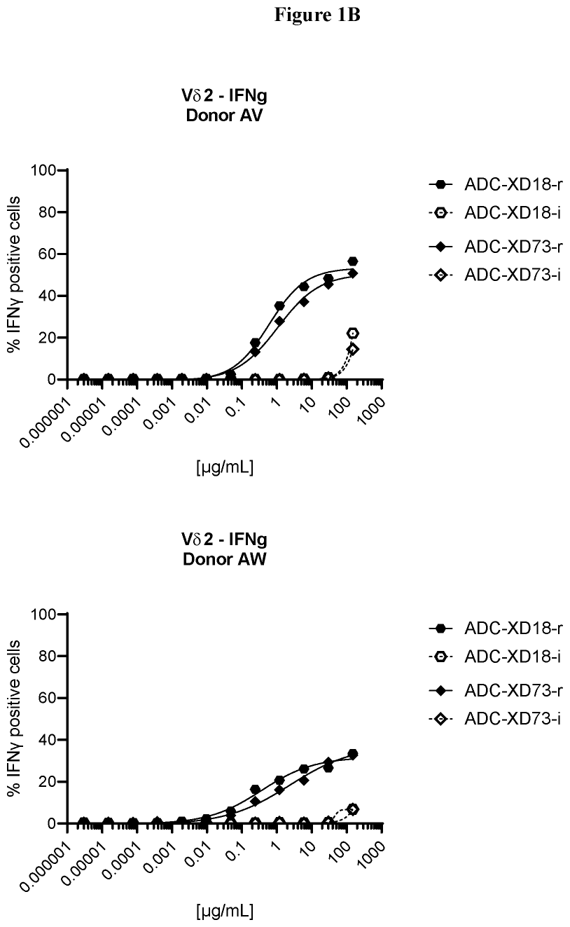

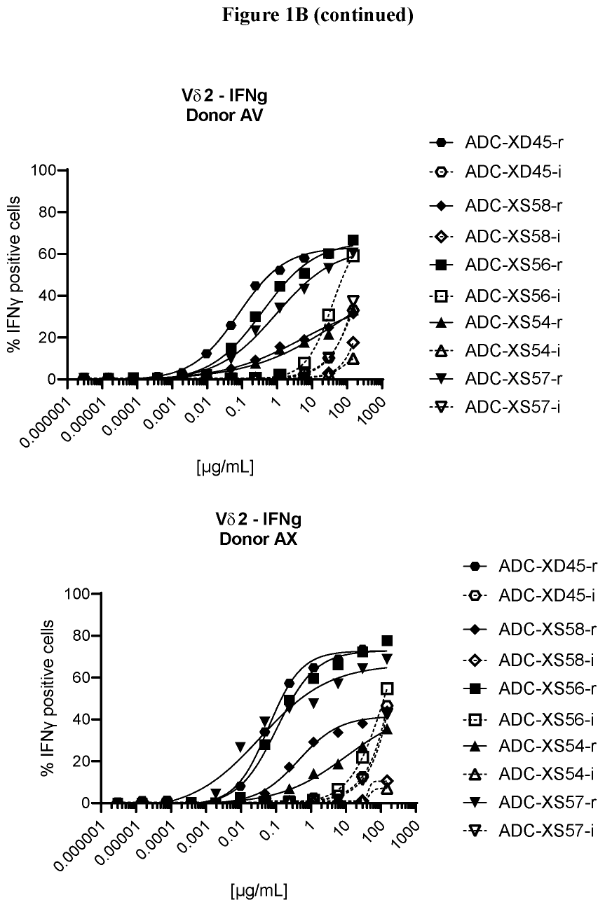

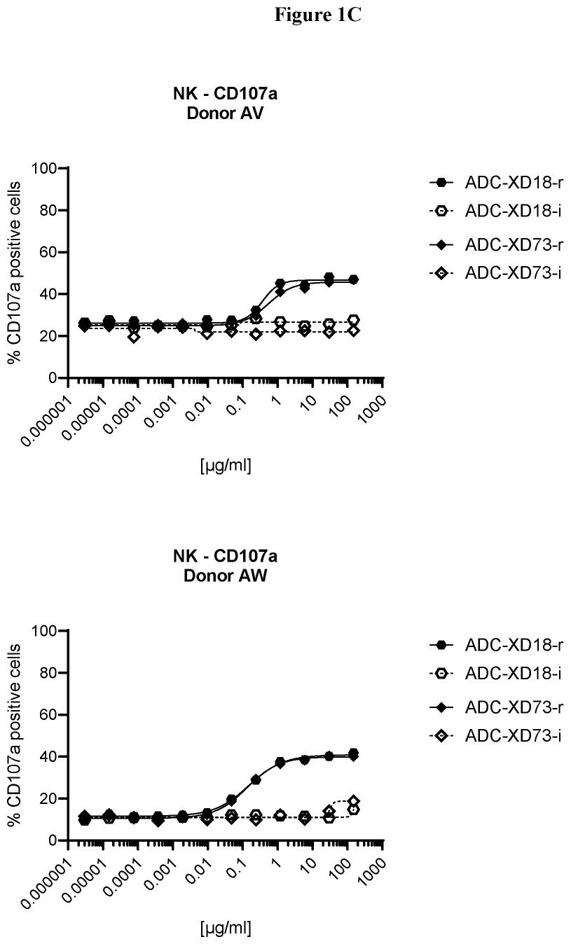

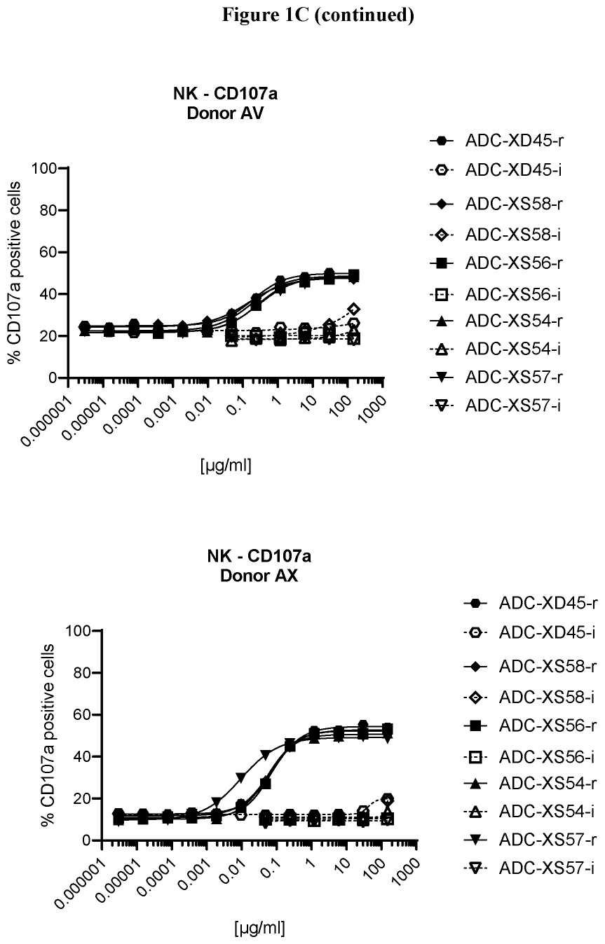

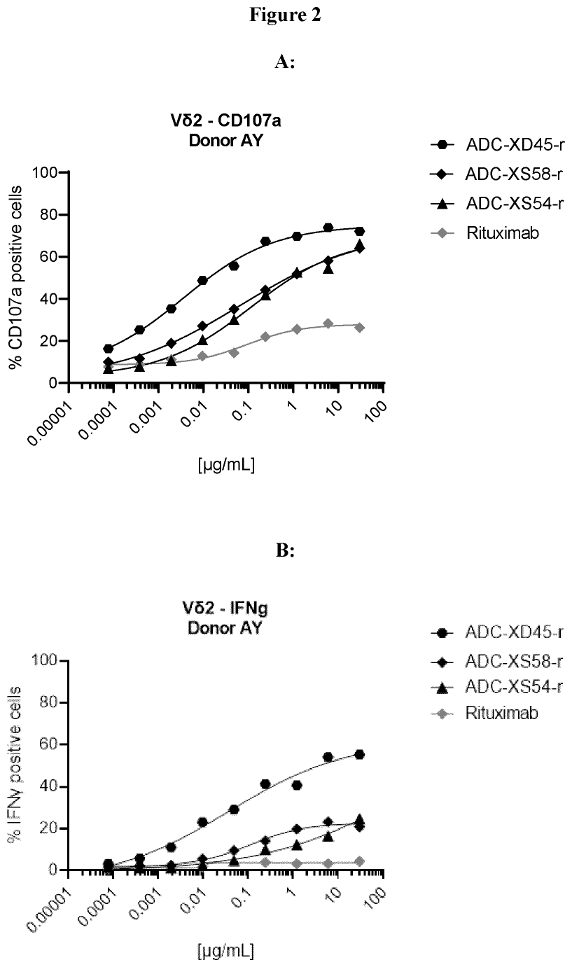

Vdelta2 gammadelta T-cell containing PBMCs. Dose-response curves for Vdelta2 gammadelta T-cell degranulation (CD107a) and IFNγ production were generated. The linker drug XD18, when conjugated to rituximab (ADC-XD18-r), was earlier described in co-pending patent application number WO2023/275025, filed in the name of Byondis B. V, and showed better potency and efficacy than rituximab. The linker drug XD73 was conjugated to rituximab (ADC-XD73-r) or a non-binding isotype control (ADC-XD73-i) and was compared to ADC-XD18-r (FIG. 1A, B and Table 3). The non-binding isotype control ADCs activated Vdelta2 gammadelta T-cells with low potency and EC₅₀ values could not be calculated reliably. ADC-XD73-r activated gammadelta T-cells with a potency and efficacy that was similar to ADC-XD18-r. The linker drug XD45, when conjugated to rituximab (resulting in ADC-XD45-r), was earlier described in WO2023/275025 and ADC-XD45-r showed better potency and efficacy then rituximab (WO2023/275025). The linker drugs XS58, XS56, XS54, and XS57 were conjugated to rituximab or a non-binding isotype control to create corresponding ADCs and were compared to ADC-XD45-r (FIG. 1A, B and Table 3). The non-binding isotype control ADCs activated Vdelta2 gammadelta T-cells with low potency and EC₅₀ values could not be calculated reliably. ADC-XS56-r and ADC-XS-57-r activated gammadelta T-cells with a potency and efficacy that was similar to ADC-XD45-r. ADC-XS58-r and ADC-XS54-r were also able to activate gammadelta T-cells. The gammadelta T-cell activation induced by ADC-XS58-r and ADC-XS54-r was more efficacious compared to the rituximab-induced gammadelta T-cell activation (FIG. 2).

In WO2023/275025, it was shown that rituximab and rituximab-ADCs induced activation of NK-cells, most likely through FcγRs that are well-known to be expressed by NK-cells (WO2023/275025). In agreement, ADC-XD18-r, ADC-XD73-r, ADC-XD45-r, ADC-XS58-r, ADC-XS56-r, ADC-XS54-r and ADC-XS57-r all induced NK-cell degranulation (i.e. CD107a) in a similar fashion (FIG. 1C).

These results show that pretreatment of tumor cells with the described CD20-binding ADCs led to dose dependent induction of IFNγ and degranulation of Vdelta2 gammadelta T-cells. The ADCs have an active Fc tail that activated NK-cells, most likely through well-defined FcγR interactions.

TABLE 2
Binding of rituximab pAg ADCs to Raji
cells, compared to rituximab.

| Compound | EC₅₀ (μg/mL) |
|---|---|
| Rituximab | 3.30 |
| ADC-XD18-r | 3.96 |
| ADC-XD73-r | 2.87 |
| ADC-XS58-r | 3.23 |
| ADC-XS56-r | 2.91 |
| ADC-XS54-r | 3.83 |
| ADC-XS57-r | 1.88 |
| ADC-XD45-r | 2.39 |

EC₅₀ values (μg/mL) represent the concentration at which 50% of the activation is achieved. A factor 2 difference in EC₅₀ is within the normal range of variation.

TABLE 3
IFNγ or CD107a production by Vdelta2 populations
induced by Raji cells pre-treated with rituximab pAg ADCs.
ADC-XD18-r

| | Donor AV | Donor AW |
|---|---|---|
| Donor | EC₅₀ | EC₅₀ |
| CD107a | 0.173 | 0.011 |
| IFNγ | 0.605 | 0.361 |

ADC-XD73-r

| | Donor AV | Donor AW |
|---|---|---|
| Donor | EC₅₀ | EC₅₀ |
| CD107a | 0.1996 | 0.027 |
| IFNγ | 1.083 | 2.343 |

ADC-XS58-r

| | Donor AV | Donor AX | Donor AY |
|---|---|---|---|
| Donor | EC₅₀ | EC₅₀ | EC₅₀ |
| CD107a | 1.277 | 0.091 | 0.067 |
| IFNγ | 0.713 | 0.449 | 0.109 |

ADC-XS56-r

| | Donor AV | Donor AX |
|---|---|---|
| Donor | EC₅₀ | EC₅₀ |
| CD107a | 0.083 | 0.019 |
| IFNγ | 0.386 | 0.107 |

ADC-XS54-r

| | Donor AV | Donor AX | Donor AY |
|---|---|---|---|
| Donor | EC₅₀ | EC₅₀ | EC₅₀ |
| CD107a | 1.001 | 0.288 | 0.114 |
| IFNγ | 66.8 | 10.18 | † |

ADC-XS57-r

| | Donor AV | Donor AX |
|---|---|---|
| Donor | EC₅₀ | EC₅₀ |
| CD107a | 0.145 | 0.003 |
| IFNγ | 0.938 | 0.035 |

ADC-XD45-r

| | Donor AV | Donor AX | Donor AY |
|---|---|---|---|
| Donor | EC₅₀ | EC₅₀ | EC₅₀ |
| CD107a | 0.022 | 0.012 | 0.004 |
| IFNγ | 0.079 | 0.061 | 0.04 |

Rituximab

| | Donor AY |
|---|---|
| Donor | EC₅₀ |
| CD107a | 0.094 |
| IFNγ | †† |

EC₅₀ values (μg/mL) represent the concentration at which 50% of the activation is achieved.
NT = not tested;
† = incomplete curve saturation (no EC₅₀ calculation possible);
†† = low number of IFNγ-positive cells, no reliable EC₅₀ calculation possible

### Example 6: Activity of Further Phosphoantigen (pAg) Conjugates (ADCs) on Gamma Delta T-Cells

More synthesized linker drug compounds were conjugated to rituximab (anti-CD20) or non-binding isotype controls. The linker drug compounds that were conjugated to rituximab are XS55, XS100-XS105, and XS107. Multiple rituximab ADCs were generated with a drug-to-antibody-ratio (DAR) of ~2 as described in previous examples. The linker-drug compounds and resulting ADCs are reflected in Table 1.

These ADCs were tested for their ability to bind Raji cells (using a binding assay, as described in Example 5) and to activate Vdelta2 gammadelta T-cells after overnight incubation with CD20-positive Raji cells. The pretreated Raji cells were cocultured with peripheral blood mononuclear cells (PBMCs) and activation (IFNγ production) and degranulation (CD107a) of Vdelta2 gammadelta T-cells and NK-cells was determined using multicolor flow cytometry, as described for the Functional assay in Example 5.

### Results/Conclusion

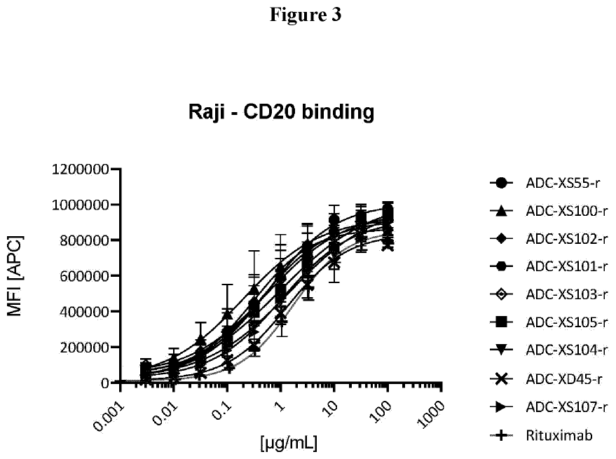

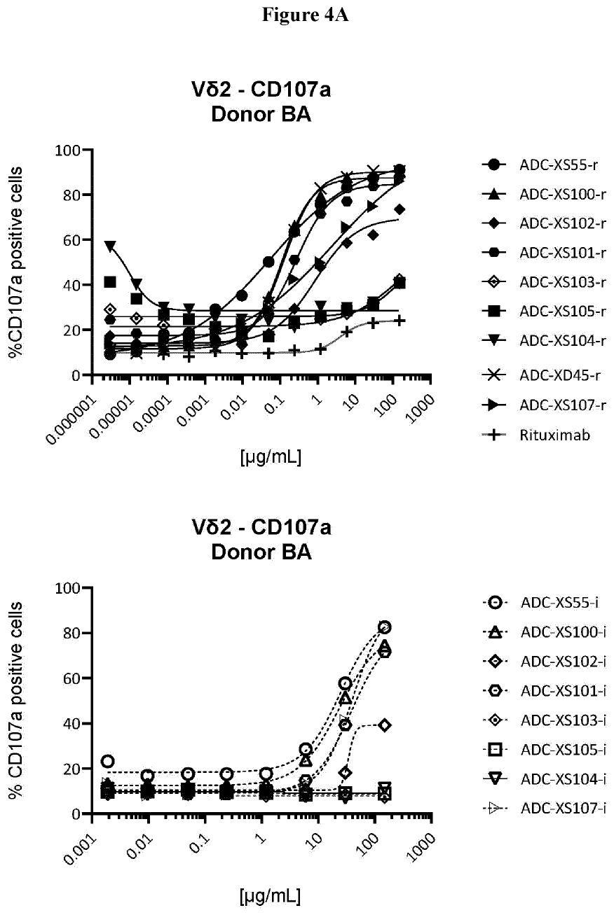

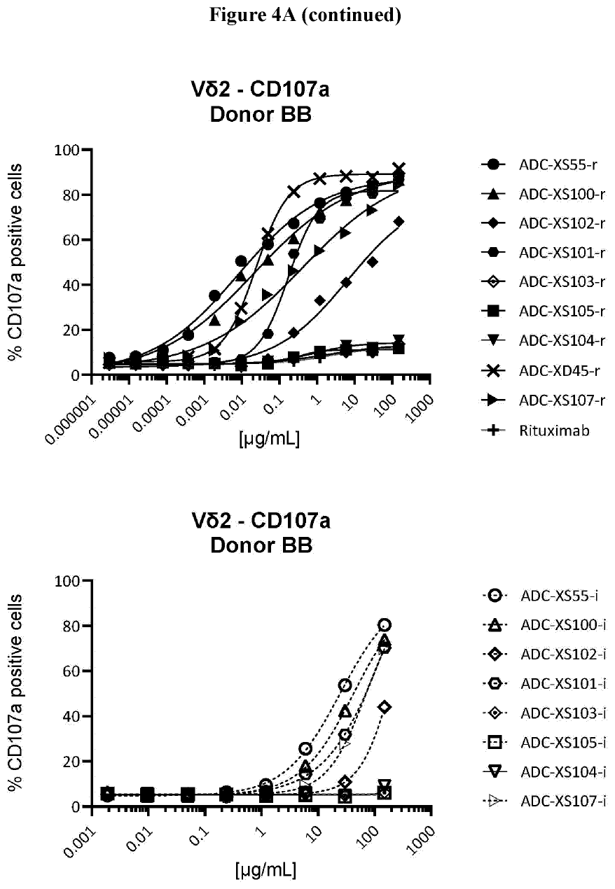

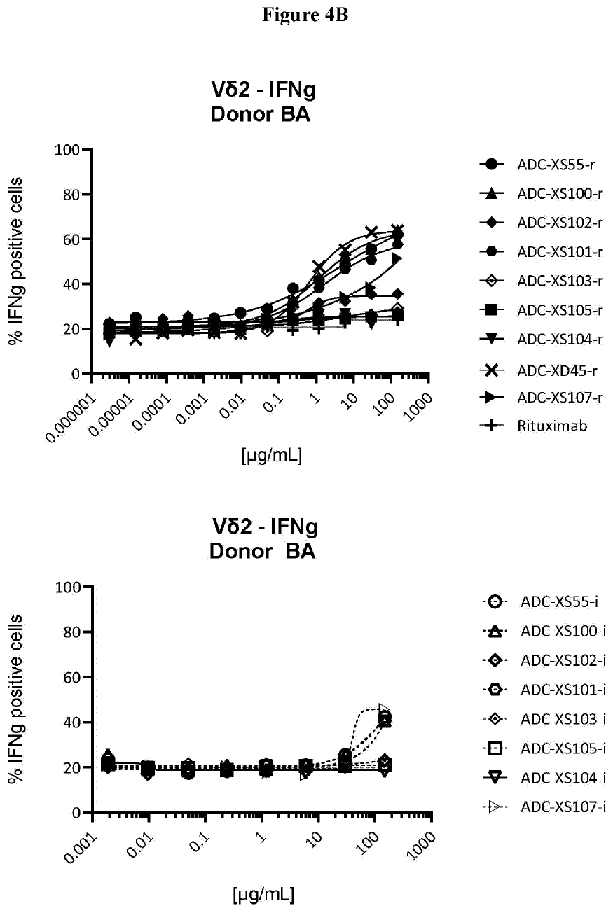

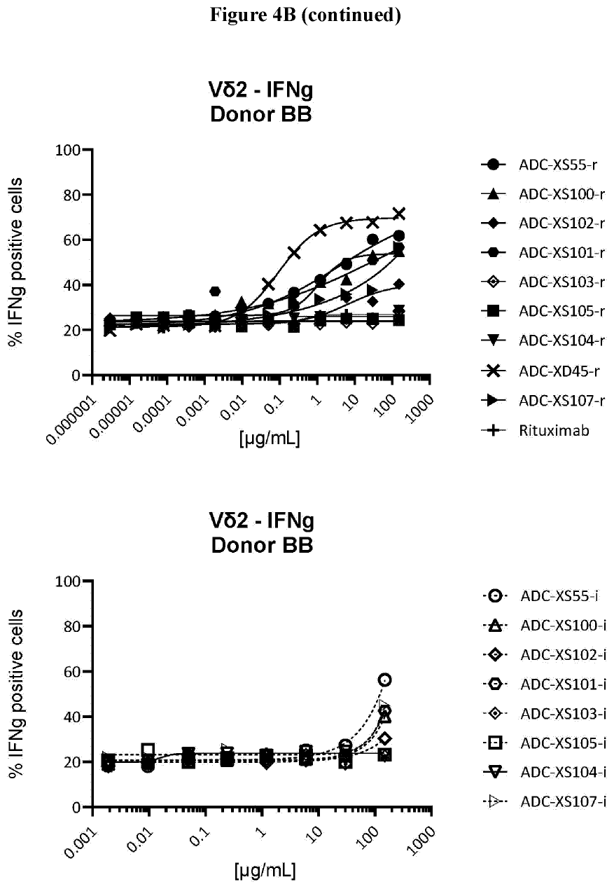

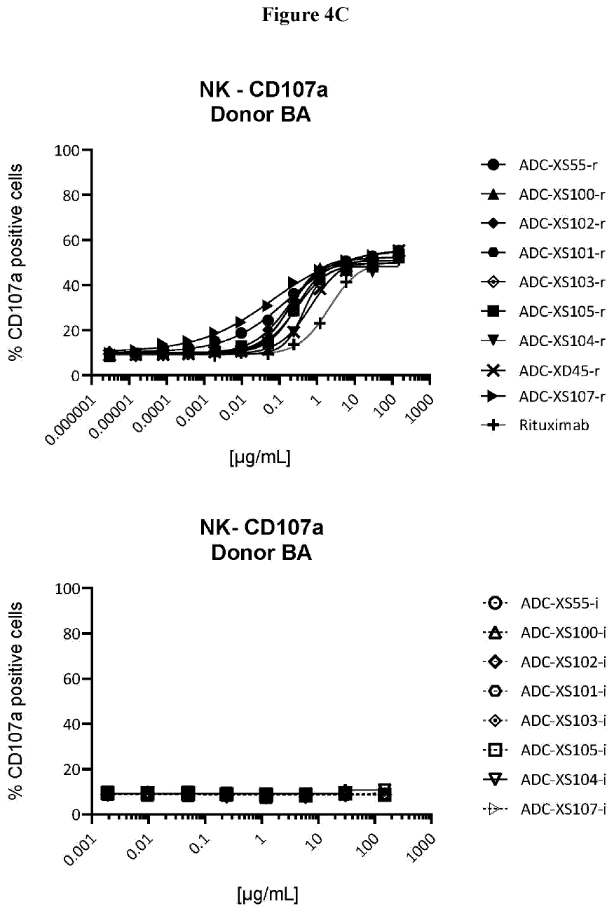

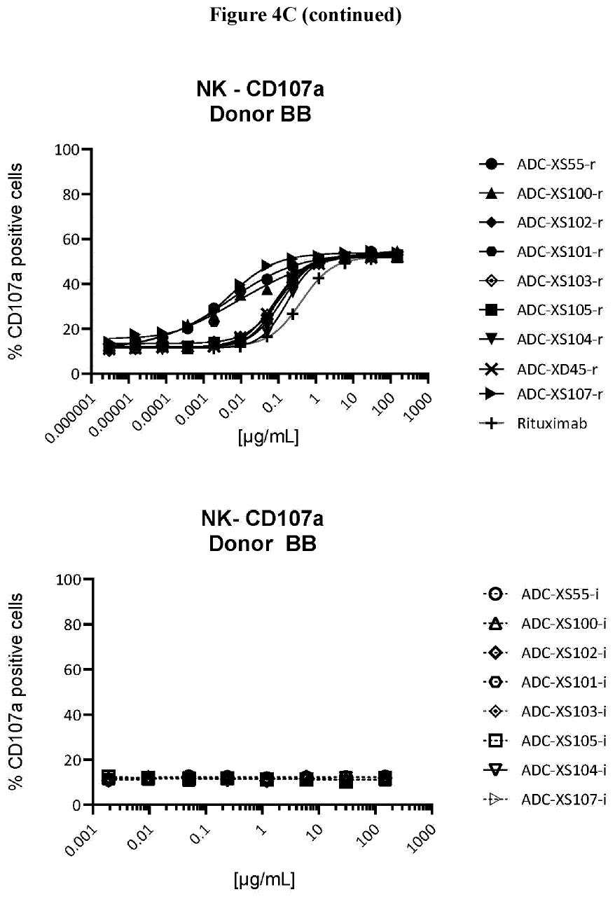

The binding to Raji cells of the rituximab ADCs is shown in FIG. 3 and all compounds showed binding. The generated ADCs were tested for their ability to induce Vdelta2 gammadelta T-cell activation after overnight incubation with Raji cells, followed by a 6 hours coculture with Vdelta2 gammadelta T-cell containing PBMCs. Dose-response curves for Vdelta2 gammadelta T-cell degranulation (CD107a) and IFNγ production were generated (FIG. 4). ADC-XS55-r, ADC-XS100-r, ADC-XS101-r, ADC-XS102-r and ADC-XS107-r induced better efficacy than rituximab. The corresponding non-binding isotype control ADCs ADC-XS55-i, ADC-XS100-i, ADC-XS101-i, ADC-XS102-i and ADC-XS107-i activated Vdelta2 gammadelta T-cells with low potency. ADC-XS103-r, ADC-XS104-r and ADC-XS105-r showed similar efficacy to rituximab. In agreement, the corresponding non-binding isotype control ADCS ADC-XS103-i, ADC-XS105-i and ADC-XS104-I also did not induce Vdelta2 gammadelta T-cell activation. All tested rituximab-ADCs (ADC-XS55-r, ADC-XS100-r, ADC-XS101-r, ADC-XS102-r, ADC-XS103-r, ADC-XS104-r, ADC-XS105-r and ADC-XS107-r) induced activation of NK-cells (FIG. 2C), most likely through FcγRs that are well-known to be expressed by NK-cells (WO2023/275025). Overall, these results show that pretreatment of tumor cells with ADC-XS55-r, ADC-XS100-r, ADC-XS101-r, ADC-XS102-r and ADC-XS107-r led to more IFNγ production and degranulation by Vdelta2 gammadelta T-cells than unconjugated rituximab. All tested ADCs have an active Fc tail that activated NK-cells, most likely through well-defined FcγR interactions.

1. A linker-drug compound with the general structure reflected in formula (I) 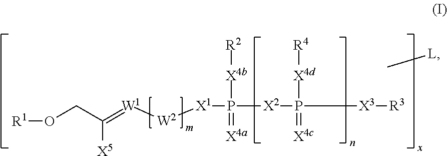
wherein
L represents a linking moiety;
W¹ is N, CH or CF, preferably CH;
W² is CH₂, CHF, CF₂ or O;
X¹ is O, S, NH, CH₂, CHF or CF₂;
X² is O, CH₂, CHF or CF₂;
X³ is absent or O or NH;
each of X^{4a-d} is independently selected from O and S;
X⁵ is
H, halogen (F, Cl, Br, I) or nitril (CN) or
ethenyl, ethynyl, ethyl, optionally substituted with one or more of the following groups: F, CH₃, CH₂F, CHF₂, CF₃, or
C₃-C₄ cycloalkyl, C₃-C₄ cycloalkenyl, allyl, propynyl, O—CH₃, S—CH₃, optionally substituted with one or more fluorine substituents, or
—CHR¹OR², CHR¹SR², CHO, CO₂R¹, CONR¹R², where R¹ and R² are independently selected from H, CH₃, CH₂F, CHF₂, CF₃, CH₂CH₃;
x is an integer ranging from 1-5;
m is 1, 2 or 3;
n is 0, 1 or 2;
R¹ is H or a connection to the linking moiety (L) or a prodrug moiety;
R² is H or a connection to the linking moiety (L) or Cat+ or a prodrug moiety;
R³ is H or a connection to the linking moiety (L) or Cat+ or a prodrug moiety;
R⁴ is H or a connection to the linking moiety (L) or Cat+ or a prodrug moiety;
or, when n is 0, R³ and R² are connected by a C₁₋₆ (hetero)alkyl group or;
when n is 1 or 2, R³ and R⁴ are connected by a C₁₋₆ (hetero)alkyl group.

2. The linker-drug compound according to claim 1, wherein X⁵ is H, Cl, F, I or Br.

3. The linker-drug compound according to claim 1, wherein W¹ is CH and R¹ is H or a connection to the linking moiety (L).

4. The linker-drug compound according to claim 1, wherein X³ is O, R³ is a connection to a linking moiety and R¹ is H.

5. The linker-drug compound according to claim 1, wherein W² is CH₂ and m is 1.

6. The linker-drug compound according to claim 1, wherein X¹ is CH₂.

7. The linker-drug compound according to claim 1, wherein X³ is O, R³ is a connection to a linking moiety, W¹ is CH, R¹ is H, W² is CH₂ and m is 1, and X¹ is CH₂.

8. The linker-drug compound according to claim 1, wherein n is 0 or 1, X^{4a-b} and X^{4c-d} (when present) are O and wherein R² and R⁴ (when present) are H.

9. The linker-drug compound according to claim 1, wherein R², R³, and R⁴ when present, are prodrug moieties selected from the group consisting of:
a pivaloyloxymethyl (POM) and isopropyloxycarbonyloxymethyl (POC) group,
a substituted or non-substituted (hetero)aryl group, and
a structure according to formula IV or V 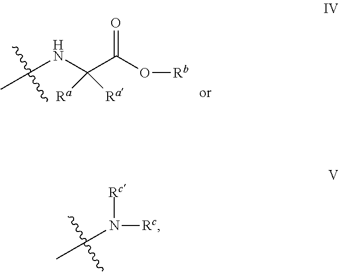
wherein;
R^{a} and R^{a′} are independently selected from H, an optionally substituted amino acid side chain and a non-polar side chain comprising an optionally substituted C₁₋₁₄ alkyl chain,
R^{b} is H, benzyl or a substituted or non-substituted (C₁₋₈)alkyl,
R^{c} and R^{c′} are independently selected from H and an, optionally substituted, C₁-C₆ alkyl, C₃-C₆ cycloalkyl, aryl or heteroaryl.

10. The linker-drug compound according to claim 9, wherein R², R³, and R⁴ when present, are independently selected from a POM- and POC-group.

11. The linker-drug compound according to claim 9, wherein n is 0, R² or R³ is a substituted or non-substituted 5 or 6 membered (hetero)aryl group and R³ is a structure according to formula IV or V, or vice versa.

12. The linker-drug compound according to claim 1, wherein the linking moiety (L) is a cleavable linking moiety.

13. The linker-drug compound according to claim 1, wherein the linking moiety (L) comprises a structure according to formula VI or VII 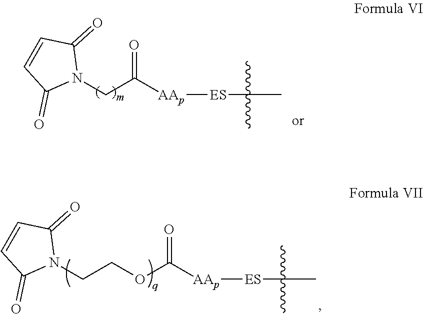
wherein
m is an integer ranging from 1 to 10, preferably 5;
AA is an amino acid, preferably a natural amino acid; and
p is 0, 1, 2, 3, or 4;
q is an integer ranging from 1 to 12, preferably 2;
ES is either absent or an elongation spacer selected from 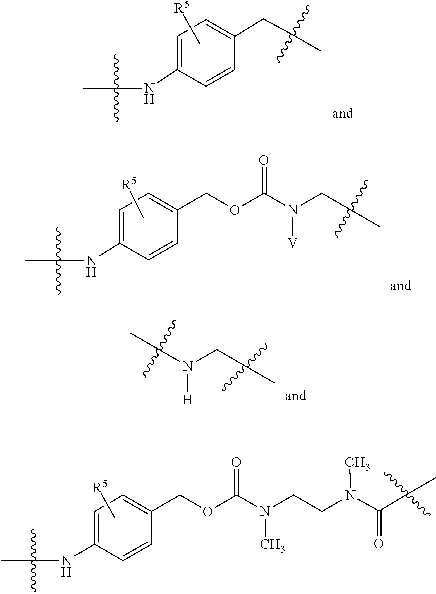
wherein R⁵ is H, halogen, CF₃, C₁₋₄ alkyl, C₂₋₄ alkenyl, C₂₋₄ alkynyl, C₁₋₄ alkoxyl, or C₁₋₄ alkylthio, preferably H, F, CH₃, CF₃, more preferably H or F, and wherein V is H, ethyl, —(CH₂CH₂O)ₚ—OMe, CH₂CH₂SO₂Me or CH₂CH₂N(Me)₂, and wherein p is an integer ranging from 1 to 12.

14. (canceled)

15. A conjugate, comprising a targeting moiety covalently linked to one or more linker-drug compounds according to claim 1.

16. The conjugate according to claim 15, wherein the targeting moiety is a tumor-targeting antibody or antigen binding fragment thereof.

17. The conjugate according to claim 15, wherein the linking moiety comprises a cleavable linker.

18. (canceled)

19. A method for the treatment of cancer, autoimmune disease or an infection, which comprises administering to a subject in need thereof a therapeutically effective amount of the conjugate according to claim 15.

20. A pharmaceutical composition comprising the conjugate according to claim 15, and one or more pharmaceutical excipients.

21. A method for the treatment of cancer in a subject, which comprises administering to a subject in need thereof a therapeutically effective amount of the conjugate according to claim 16.
